SEQUENCE LISTING (31 sequences)

SEQ ID NO: 1 - DNA, 696 bp, Artificial Sequence
  CNS-1 promoter
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatggggctgggcataaaagtcagggcagagccatctattgcttacatttgcttctg

SEQ ID NO: 2 - DNA, 709 bp, Artificial Sequence
  CNS-2 promoter
ggtgtgtggaagggtgagaggcacacacacagacactgaaagaatcctaggcctggtaggcacttaacaaatgtctgttacagaccagaattttattgctgttagagacccaagcccctcataggaacagtgagaaacaggtgcagaaaggcggagtaactttatctaaagtcataggctccctgaatagcagagctgacacctacaaggaagcgttggagaccagatctaccagctagcctccctgagaccacgaggtggcgccgcagcaccggctgtggccgatgccagccaggtagccggtttcccacgtcccccgcacgcacgcacctctttgctgcaggaatcccgggctgccccgacctggagtagggggggtggtgagtgggactgagtccctagaagcctggaccctcacttcgttcctgtacatccagctcgcctgtagacagtgggggaggatgaagggaagaggactcaagcgcaactttgaatcatcacgccttcgacagtccgcgcacgtttatttcatttatctttgaaaacgagggaggggaagcctggagaaggcgggatgggccaagggtgagttggcccccggggagctggtccctgttcctggctttagtcccaggggcgcggtctgtgtgtagggcgggctgggcataaaagtcagggcagagccatctattgcttacatttgcttctg

SEQ ID NO: 3 - DNA, 768 bp, Artificial Sequence
  CNS-3 promoter
tgctaccagagccgggagagctgctcggagacgcctccggggtgcgggctggacatgagcagcggctgccggtcctgggactaggccccgccattttggatccgctgacaggtcagcgaagtctcttcctagagttccggtgtcgtgaaggccgccctgacatcgcaatagggaattagtgggaagggcccttaaattgggcgagccaaggtggggggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcc

SEQ ID NO: 4 - DNA, 736 bp, Artificial Sequence
  CNS-4 promoter
aaggagaatggtaaacagcaggagcgaagcggctgaggagaaagaagaggaaagaaaggcgagacgtgggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcccgagcggagaggcggcccgggagcaggggggcggcccccactccggccgggtgcccggcccctggcccctgcctgccctctagatcgccgccgcagccgccgctactgggagtctg

SEQ ID NO: 5 - DNA, 1744 bp, Artificial Sequence
  CNS-5 v2 promoter
ttgtaatgggaataagggcaggactcctgggtataagtagctcagctgatcccaccctgcttctatgtgttaattcatttattcattcattcaacaagcatttgttgaaatgctctttgtgtcaggctcagcaggaagcagtggcaataaaatggtgaacaagaaagactcggggtttcttcatctatgttgatgtctgcagagaacagtatcagccttctaggaagtttgtaatcagatacattgttagagagatacttatctagtaaattcctactcatcctataaggctcaaaacaaatgcctctatgaaaccttccgtgattccctcaggcagagttaagagcttcctttcctgggcctctatctccttccattagtattataactgtttaccagtttcccctctagactaaatttctcaaaagagagaatgaggtctctttcagtcttctttgcatctttaaactagcctgggtcccagcctgtttgatgaaagaaacaagaacactgatacaagccacagccccttggcaaaaaagatacccaatagcaatggcaatgtaaaatcagttttagtaaatgaatcaagaattctgatgctttagggaaagtaatgtgaacctggcaccattaacaaattcagaactcttcttcttaggagctctctaactgaacagacagagggatgtcaacccctaattcagcttgatcgtatctcagcaactacatttaatgagacagtgggaaaaagagagctgtccacttttaaatcagcatatttctaactaaacaatggcaatggctaaatctttaaaatgcctatttctctcaagaacactgcaatggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaaacatgagatgccaggaaaacacacttaaaaaaaaaattcccactttaagctttagactgaatgtgagaaaggagatgataaaaagagtatcacaagagaatcttcaggctgtggg

SEQ ID NO: 6 - DNA, 1104 bp, Artificial Sequence
  CNS-6 v2 promoter
tttggcactgtgagcagtttacttgacaaattctgtcaaatatttgctttctgaaatctcgagaattggttgaatataattgtacttaatgtttgcaaaataaataaatatgggactaaggacgttctatcattaatttgtcagaaaagagagttgtcatttctgaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgatttccagcagagttctattaaagggaggacagaatctgtctcttccaaggtggaaaatcgtgaatattccctgcattaatgaaccaagttaacactttaattgcttatagaaccgagttctccaatgacagcattaaaagatagggaggctctgatttatggtcaacacagatttgtaaccc

SEQ ID NO: 7 - DNA, 1910 bp, Artificial Sequence
  CNS-7 v2 promoter
tcaacatggataaccaaagttcttaaaactacgctttcaatgaacacatatcctttgagcaagactaataatgaggaatgggagccagctcctgtgatatttatgcaactactaaattctcactgaagtcaatgggagtttgcttacgtaagggctgcaaactttagcctccagagattaaaggggaaaaaaatccttaaactctttcaacattaatattgcctgtaaggaatccagccatgacctaagccatggagctttctgaacctagcaagtagaagggtaaacagtaaacaccagttattttaagcacaatctaatcagagttcaatgagaagcaatattatatttgatctctaaggtattaatacttgtatatcactattagacatctttatgtagtccattatccaaacaatggcttaagtctgtggtatttaataaatcaagtttccatggccgtgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccatagtgatatttaaaaaagaaactttggcaaatgcttgcaaaaaacacaccttttacaattttaaatgtgatttactgatggccagaacttgttaaacatagtaggaaattaaatatttattcatcttatttcattttcagggccgtaaacgctccttctgagtcattcccaataacaagaatttctaccagtaaagctattaacaggcatcaaaataggggagtgctaaattaagatgagattgtaaaagcaaataagaacatacgcagactcgcataggagtgcaaatgatcgtttctgattgaaatgtttatagctaaatgagtttggctgaattaaacacaaatgttccaaaagataagccgtagctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgctatgcttcaattttcatctaggtgatcaagattcattttcttgttctgtcatccaaataggcagacagaaaagtgattgaaatacattatggagatgtgtcattgcaca

SEQ ID NO: 8 - DNA, 540 bp, Artificial Sequence
  CNS-8 v2 promoter
gctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgc

SEQ ID NO: 9 - DNA, 643 bp, Artificial Sequence
  CRE0004_Lmx1b
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatg

SEQ ID NO: 10 - DNA, 656 bp, Artificial Sequence
  CRE0003_Pitx3
ggtgtgtggaagggtgagaggcacacacacagacactgaaagaatcctaggcctggtaggcacttaacaaatgtctgttacagaccagaattttattgctgttagagacccaagcccctcataggaacagtgagaaacaggtgcagaaaggcggagtaactttatctaaagtcataggctccctgaatagcagagctgacacctacaaggaagcgttggagaccagatctaccagctagcctccctgagaccacgaggtggcgccgcagcaccggctgtggccgatgccagccaggtagccggtttcccacgtcccccgcacgcacgcacctctttgctgcaggaatcccgggctgccccgacctggagtagggggggtggtgagtgggactgagtccctagaagcctggaccctcacttcgttcctgtacatccagctcgcctgtagacagtgggggaggatgaagggaagaggactcaagcgcaactttgaatcatcacgccttcgacagtccgcgcacgtttatttcatttatctttgaaaacgagggaggggaagcctggagaaggcgggatgggccaagggtgagttggcccccggggagctggtccctgttcctggctttagtcccaggggcgcggtctgtgtgtagggc

SEQ ID NO: 11 - DNA, 215 bp, Artificial Sequence
  CRE0002_Gbf1
tgctaccagagccgggagagctgctcggagacgcctccggggtgcgggctggacatgagcagcggctgccggtcctgggactaggccccgccattttggatccgctgacaggtcagcgaagtctcttcctagagttccggtgtcgtgaaggccgccctgacatcgcaatagggaattagtgggaagggcccttaaattgggcgagccaaggtggg

SEQ ID NO: 12 - DNA, 53 bp, Artificial Sequence
  SYNP_CRE151
gggctgggcataaaagtcagggcagagccatctattgcttacatttgcttctg

SEQ ID NO: 13 - DNA, 553 bp, Artificial Sequence
  CRE0001v1_Pitx3
gggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcc

SEQ ID NO: 14 - DNA, 467 bp, Artificial Sequence
  Synapsin-1
gagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcg

SEQ ID NO: 15 - DNA, 20 bp, Artificial Sequence
  GFP forward primer
ggcacaagctggagtacaac

SEQ ID NO: 16 - DNA, 20 bp, Artificial Sequence
  GFP reverse primer
agttcaccttgatgccgttc

SEQ ID NO: 17 - DNA, 25 bp, Artificial Sequence
  GFP probe
  misc_feature (1)..(1): FAM
agccacaacgtctatatcatggccg

SEQ ID NO: 18 - DNA, 19 bp, Artificial Sequence
  mouse GAPDH forward primer
acggcaaattcaacggcac

SEQ ID NO: 19 - DNA, 20 bp, Artificial Sequence
  mouse GAPDH reverse primer
tagtggggtctcgctcctgg

SEQ ID NO: 20 - DNA, 24 bp, Artificial Sequence
  mouse GAPDH probe
  misc_feature (1)..(1): VIC
ttgtcatcaacgggaagcccatca

SEQ ID NO: 21 - DNA, 974 bp, Artificial Sequence
  CNS-1 + CMV-IE UTR and intron
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatggggctgggcataaaagtcagggcagagccatctattgcttacatttgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcag

SEQ ID NO: 22 - DNA, 868 bp, Artificial Sequence
  CNS-4+ CMV-IE UTR and intron
aaggagaatggtaaacagcaggagcgaagcggctgaggagaaagaagaggaaagaaaggcgagacgtgggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgctcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcag

SEQ ID NO: 23 - DNA, 835 bp, Artificial Sequence
  CNS-5 promoter
ggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaagggctgggcataaaagtcagggcagagccatctattgcttacatttgcttctg

SEQ ID NO: 24 - DNA, 812 bp, Artificial Sequence
  CNS-6 promoter
gaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgatgggctgggcataaaagtcagggcagagccatctattgcttacatttgcttctg

SEQ ID NO: 25 - DNA, 487 bp, Artificial Sequence
  CNS-7 promoter
tgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccatagggctgggcataaaagtcagggcagagccatctattgcttacatttgcttctg

SEQ ID NO: 26 - DNA, 593 bp, Artificial Sequence
  CNS-8 promoter
gctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgcgggctgggcataaaagtcagggcagagccatctattgcttacatttgcttctg

SEQ ID NO: 27 - DNA, 284 bp, Artificial Sequence
  CMV-IE 5′ UTR and intron
tcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcag

SEQ ID NO: 28 - DNA, 782 bp, Artificial Sequence
  CRE0005_faf1_short
ggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaa

SEQ ID NO: 29 - DNA, 759 bp, Artificial Sequence
  CRE0006_Pitx2_short
gaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgat

SEQ ID NO: 30 - DNA, 434 bp, Artificial Sequence
  CRE0007_Pitx2_short
tgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccata

SEQ ID NO: 31 - DNA, 540 bp, Artificial Sequence; identical to SEQ ID NO: 8
  CRE0008_Pitx2_short

(19) United States
(12) Patent Application Publication
(10) Pub. No.: US 2023/0143758 A1
(43) Pub. Date: May 11, 2023

(54) REGULATORY NUCLEIC ACID SEQUENCES

(71) Applicant: ASKLEPIOS BIOPHARMACEUTICAL, INC., Research Triangle Park, NC (US)

(72) Inventors: Tony Oosterveen, Midlothian (GB); Anne Braae, Midlothian (GB); Sinclair Cooper, Midlothian (GB); Juan Manuel Iglesias, Midlothian (GB); Michael L. Roberts, Midlothian (GB)

(73) Assignee: ASKLEPIOS BIOPHARMACEUTICAL, INC., Research Triangle Park, NC (US)

(21) Appl. No.: 17/919,710

(22) PCT Filed: Apr. 19, 2021
(86) PCT No.: PCT/GB2021/050939

§ 371 Date: Oct. 18, 2022

(30) Foreign Application Priority Data
Apr. 20, 2020 (GB) 2005732.9

(51) Int. Cl. A61K 48/00; C12N 15/86; C07K 14/47

(52) U.S. Cl. CPC A61K 48/0058; C12N 15/86; C07K 14/4705; C12N 2750/14143; C12N 2740/15043; C12N 2830/008

(57) ABSTRACT

The present invention relates to regulatory nucleic acid sequences, in particular CNS—specific promoters, and elements thereof. The invention also relates to expression constructs, vectors, virions, pharmaceutical compositions and cells comprising such promoters and to methods of their use. The regulatory nucleic acid sequences are of particular utility for gene therapy applications.

### CROSS REFERENCE TO RELATED APPLICATIONS

This application is a 35 U.S.C. § 371 National Phase Entry Application of International Application No. PCT/GB2021/050939 filed Apr. 19, 2021, which claims benefit under 35 U.S.C. § 119(b) of GB Application No. 2005732.9 filed Apr. 20, 2020, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Apr. 23, 2021, is named pctgb2021050939-seql and is 29 KB in size.

### FIELD OF THE INVENTION

The present invention relates to regulatory nucleic acid sequences, in particular CNS-specific promoters, and elements thereof. The invention also relates to expression constructs, vectors, virions, pharmaceutical compositions and cells comprising such promoters and to methods of their use. The regulatory nucleic acid sequences are of particular utility for gene therapy applications.

### BACKGROUND OF THE INVENTION

The following discussion is provided to aid the reader in understanding the disclosure and does not constitute any admission as to the contents or relevance of the prior art.

Following extensive study of the internal mechanisms of gene regulation within the body, research focus has recently shifted to regulation of gene expression by introducing exogenous nucleic acid sequences into cells.

This is done conventionally in research and bioprocessing, wherein the nucleic acid sequence of a desired expression product operably liked to a promoter is introduced into a production cell line, often in the form of a vector.

In the field of gene therapy, this has been of particular interest for single gene disorders or Mendelian disorders which are caused by the presence of a faulty gene into the cells of a patient. Introduction of the nucleic acid sequence of a wild type allele of the faulty gene operably linked to a promoter into the cells of a patient is a favourable treatment option as it can, in theory, cure the condition while conventional medicines can only address the symptoms.

In gene therapy, controlling the expression of the exogenous nucleic acid which has been introduced into the cells is of paramount importance for the health and safety of the patients. The level of an expression product not only needs to be within a therapeutic window but also the expression needs to be within a required tissue or in a specific region within the required tissue. Expression outside the therapeutic window (i.e. lower or higher) or expression outside the therapeutic region, or even outside the specific region within the required tissue, may not be useful therapeutically or even be deleterious.

Dopamine transporter deficiency syndrome, a type of childhood parkinsonism, is a candidate for gene therapy by introducing a replacement gene as it is caused by loss-of-function mutation in a single gene, DAT1/SLC6A3 (Kurian, et al., 2009). DAT1/SLC6A3 encodes a pre-synaptic dopamine transporter which is involved in the translocation of extraneuronal dopamine into dopaminergic neurones. The dopamine transporter transports dopamine, two sodium ions and one chloride ion into the cell using the driving force of the sodium gradient across the plasma membrane. As a result, DAT1/SLC6A3 plays a role in modulating the duration and intensity of dopamine signalling (Ng, et al., 2014) and its malfunction is associated with a variety of neuropsychiatric disorders such as attention deficit hyperactivity disorder (Kurian, et al., 2009).

A particular difficulty in introducing a replacement DAT1/SLC6A3 gene is that, in non-disease state, DAT1/SLC6A3 is specifically expressed in the midbrain, as shown in FIG. 1A. In order to best mimic the native expression of DAT1/SLC6A3, it is desirable to ensure that the replacement DAT1/SLC6A3 gene is expressed within the midbrain (as this is the location of the dopaminergic neurones) but it is also preferable that the expression in other parts of the brain is minimal.

Therefore, there is a need for promoters driving expression in the midbrain among other CNS regions, as well as promoters which drive expression specifically in the dopaminergic neurones in midbrain.

Angelman syndrome is also a candidate for gene therapy by introducing a replacement gene. Angelman syndrome is most commonly caused by mutation or absence of a single gene, UBE3A. UBE3A is involved in targeting proteins for degradation. In most neurones only the copy of the UBE3A gene inherited from the mother is active and loss of the maternal UBE3A gene leads to Angelman syndrome.

A particular difficulty in introducing a replacement UBE3A gene is that, in non-disease state, UBE3A is widely expressed in the brain as shown in FIG. 1B. In order to best mimic the native expression of UBE3A gene, it is preferable that the replacement UBE3A gene is widely expressed in the brain.

Therefore, there is also a need for promoters driving expression in many or all regions of the brain (e.g. pan-CNS).

Other diseases of the CNS are suitable targets for gene therapy, and in some such diseases targeted expression of a therapeutic gene in a specific CNS tissue may be desired, and in others more generalised, non-specific expression in the CNS may be suitable.

One or more aspects of the present invention are intended to address one or more of the above-mentioned problems.

### SUMMARY OF THE INVENTION

In a first aspect of the present invention, there is provided a synthetic central nervous system (CNS)-specific promoter comprising or consisting of a sequence according to any one of SEQ ID NOs 1-8, 21-26 or a functional variant thereof.

In some embodiments the synthetic CNS-specific promoter comprises or consists of a sequence which is at least 60%, 65%, 70%, 75%, 80%, 85%, 90%, 95%, 96%, 97%, 98% or 99% identical to any one of SEQ ID NOs 1-8, 21-26.

The present invention thus provides various synthetic CNS-specific promoters and functional variants thereof. It is generally preferred that a promoter according to the present invention which is a variant of any one of SEQ ID NO 1-8, 21-26 retains at least 25%, 50%, 75%, 80%, 85%, 90%, 95% or 100% of the activity of the reference promoter. Suitably said activity is assessed using the examples as described herein, but other methods can be used.

In another aspect of the present invention, there is provided a CNS-specific cis-regulatory element (CRE) comprising or consisting of a sequence according to any one of SEQ ID NOs: 9-11, 28-31, or a functional variant of any thereof. In some embodiments the CNS-specific CRE comprises a sequence which is at least 60%, 65%, 70%, 75%, 80%, 85%, 90%, 95%, 96%, 97%, 98% or 99% identical to any one of SEQ ID NOs 9-11, 28-31.

It is generally preferred that a CNS-specific CRE according to the present invention which is a variant of any one of SEQ ID NOs 9-11, 28-31 retains at least 25%, 50%, 75%, 80%, 85%, 90%, 95% or 100% of the activity of the reference CRE. Retention of activity can be assessed by comparing expression of a suitable reporter under the control of the reference promoter with an otherwise identical promoter comprising the substituted CRE under equivalent conditions. Suitably said activity is assessed using the examples as described herein, but other methods can be used.

Suitably, the CRE according to the present invention may be combined with additional CREs to form a cis-regulatory module (CRM). Suitably, the additional CREs may be CREs according to SEQ ID NOs 9-11, 28-31 or functional variants thereof, or they can be other CREs. Suitably, the additional CREs are CNS-specific.

In another aspect of the present invention there is provided a synthetic CNS-specific promoter comprising or consisting of a CRE according to any one of SEQ ID NOs 9-11, 28-31 or a functional variant thereof. In some embodiments, the CRE may be operably linked to a promoter element. In some embodiments, the promoter element may be a minimal or a proximal promoter. Preferably, the proximal promoter is a CNS-specific proximal promoter.

In a further aspect of the present invention, there is provided a minimal or proximal promoter comprising or consisting of a sequence according to any one of SEQ ID NOs: 12-13, or a functional variant thereof. In another aspect of the present invention, there is provided a synthetic promoter comprising said minimal or proximal promoter, suitably a synthetic CNS-specific promoter comprising said minimal or proximal promoter. Suitably a functional variant of the minimal or proximal promoter comprises a sequence which is at least 60%, 65%, 70%, 75%, 80%, 85%, 90%, 95%, 96%, 97%, 98%, 99% identical to SEQ ID NO: 12-13.

Suitably, any one of CNS-4, CNS-5_v2, CNS-6_v2, CNS-7_v2, CNS-8_v2 (SEQ ID NO: 4-8) can function as a minimal or proximal promoter. Therefore, there is provided a synthetic CNS-specific promoter comprising a minimal or proximal promoter according to any one of SEQ ID NOs: 12-13 or SEQ ID NO: 4-8. Suitably the minimal or proximal promoter can be operably linked with a CRE or CRM. The CRE may be a CRE according to this invention or any other CRE. The CRM may comprise a CRE according to this invention. Suitably, the CRE or the CRM is CNS-specific.

The CREs, minimal/proximal promoters or promoters of the present invention can be active in specific region of the CNS, preferably in a specific region in the brain, or in specific brain cell type or cell types or in a combination of both.

The CREs, minimal/proximal promoters or promoters of the present invention can be active in one or more of the various parts of the CNS. The CNS consists primarily of the brain and the spinal cord. The retina, optic nerve, olfactory nerves, and olfactory epithelium are sometimes considered to be part of the CNS alongside the brain and spinal cord. This is because they connect directly with brain tissue without intermediate nerve fibres. Suitably, the CREs, minimal/proximal promoter or promoters of the present invention may be active in the brain and the spinal cord. Suitably, the CREs, minimal/proximal promoter or promoters of the present invention may be active in the brain but not in the spinal cord or any other part of the CNS. Suitably, the CREs, minimal/proximal promoter or promoters of the present invention may be active in the spinal cord but not in the brain. Preferably the CREs, minimal/proximal promoter or promoters of the present invention may be active in the brain. Suitably the CREs, minimal/proximal promoter or promoters of the present invention may be active in one or more of the various areas within the brain.

Non-limiting examples of brain areas include: frontal lobe, pariental lobe, occipital lobe, temporal lobe (which includes the hippocampus and amygdala), cerebellum, midbrain, pons, medulla and the diencephalon (which includes the thalamus and hypothalamus). Nonlimiting examples of spinal cord areas include: cervical vertebrae, thoracic vertebrae, lumbar vertebrae, sacrum vertebrae and coccyx vertebrae. In some embodiments, it may be desirable that the CRE, minimal/proximal promoter or promoter of the present invention shows widespread activity in the brain. In some embodiments, the CRE, minimal/proximal promoter or promoter of the present invention is active in all parts of the brain or CNS (pan-CNS), preferably in all areas of the brain. In some embodiments the CRE, minimal/proximal promoter or promoter of the present invention is active in the brain but not in other parts of the CNS e.g. the spinal cord. In some embodiments, the CRE, minimal/proximal promoter or promoter of the present invention is active in 1, 2, 3, 4, 5, 6, 7, 8 or 9 of the areas of the brain recited above. In some embodiments, the CRE, minimal/proximal promoter or promoter of the present invention is active in the majority of the areas in the brain, i.e. at least 5, at least 6, at least 7, at least 8 or all 9 of the 9 areas of the brain recited above. In some embodiments, the CRE, minimal/proximal promoter or promoter of the present invention is active in from 4 to 6 areas of the brain recited above. In some embodiments, the CRE, minimal/proximal promoter or promoter of the present invention is active in from 2 to 4 areas of the brain recited above, such as for example the midbrain, temporal lobe and diencephalon. In some embodiments, the CRE, minimal/proximal promoter or synthetic promoter of the present invention may be active in the abovementioned areas of the brain and the spinal cord. In some embodiments the CRE, CRM, minimal/proximal promoter or synthetic promoter of the present invention is active in the spinal cord but not in other parts of the CNS, e.g., the brain. In some embodiments, the CRE, CRM, minimal/proximal promoter or synthetic promoter of the present invention is active in 1, 2, 3, 4, or 5 of the areas of the spinal cord recited above. In some embodiments, the CRE, CRM, minimal/proximal promoter or promoter of the present invention is active in the majority of the areas in the spinal cord, i.e. at least 3, at least 4 or all 5 of the 5 areas of the spinal cord recited above.

In some embodiments, it may be desirable that the CRE, minimal/proximal promoter or promoter of the present invention shows predominant activity in one area of the CNS, suitably in one area of the brain. Suitably, it may be desirable that the CRE, minimal/proximal promoter or promoter of the present invention shows activity in one area of the brain but no, or only minimal, activity in the rest of the brain or CNS. In some embodiments, the CRE, minimal/proximal promoter or promoter of the present invention is active in only one area of the CNS areas of the brain recited above, such as the midbrain. In some preferred embodiments, the CRE, minimal/proximal promoter or promoter of the present invention is specifically active in the midbrain (midbrain-specific). In one preferred embodiment, the CRE, minimal/proximal promoter or promoter of the present invention is specifically active in the midbrain (midbrain-specific) but shows no or only minimal activity in other areas of the brain.

The CREs, minimal/proximal promoters or promoters of the present invention can be active in various cells of the CNS. The predominant cell types in the brain are neurones, astrocytes, oligodendrocytes, microglia, and ependymal cells. Other cell types may be present, particularly in inflammatory condition. In some embodiments, it may be desirable for the promoter to be active in many different cell types. In some embodiments, the CRE, minimal/proximal promoter or promoter of the present invention is active in substantially all cells of the CNS (e.g. neurones, astrocytes, oligodendrocytes, microglia, ependymal cells). In some embodiments, the CRE, minimal/proximal promoter or promoter of the present invention is active in at least four CNS cell types from the CNS cell types listed above, such as neurones, astrocytes, microglia and oligodendrocytes. In some embodiments, the CRE, minimal/proximal promoter or promoter of the present invention is active in at least three CNS cell types from the CNS cell types listed above, such as neurones, astrocytes and oligodendrocytes.

In some embodiments, it may be desirable for the promoter to be active in a limited number of CNS cell types, or in not more than one CNS cell type. In some embodiments, the CRE, minimal/proximal promoter or promoter of the present invention is active in no more than 4, 3, 2 or 1 of CNS cell types from the CNS cell types listed above. In some embodiments, the CRE, minimal/proximal promoter or promoter of the present invention is active in no more than two CNS cell types from the CNS cell types listed above, such as neurones, and oligodendrocytes. In some embodiments, the CRE, minimal/proximal promoter or promoter of the present invention is active in only one CNS cell type from the CNS cell types listed above, such as neurones.

In some embodiments, the CRE, minimal/proximal promoter or promoter of the present invention is active in specific subtypes of CNS cell, such as for example dopaminergic neurones. In some specifically preferred embodiments, the CRE, minimal/proximal promoter or promoter of the present invention is active in dopaminergic neurones. In some preferred embodiments, the CRE, minimal/proximal promoter or promoter of the present invention is active in dopaminergic neurones but not in other CNS cell types or other CNS cell subtypes. In some preferred embodiments, the CRE, minimal/proximal promoter or promoter of the present invention is active in GABAergic or glutamatergic neurones. In some embodiments, the CRE, minimal/proximal promoter or promoter of the present invention is active in a specific type of CNS cell or subtype of CNS cell, and in a specific area of the brain.

The CRE, minimal/proximal promoter or promoter of the present invention may or may not be active in tissues outside the CNS. Non-limiting examples of tissues outside the CNS are: the heart, the liver, the kidney, skeletal muscles and the spleen. Suitably, in some embodiments, the CRE, minimal/proximal promoter or promoter of the present invention is not or is minimally active in tissues or cells outside of the CNS. Suitably, the CRE, minimal/proximal promoter or promoter of the present invention is active in no more than 1, 2, 3, 4 tissues out of the tissues outside of the CNS described above in ICV delivery. Suitably, the CRE, minimal/proximal promoter or promoter of the present invention is active in no more than 1, 2, 3, 4 tissues out of the tissues outside of the CNS described above in IV delivery.

Suitably, in some embodiments, it may be desirable for the CRE, minimal/proximal promoter or promoter of the present invention to be active in the CNS but to also have activity in other tissues outside of the CNS. Suitably, the CRE, minimal/proximal promoter or promoter of the present invention may be active in at least 1, 2, 3, 4 or 5 of the tissues outside of the CNS described above in ICV delivery. Suitably, the CRE, minimal/proximal promoter or promoter of the present invention may be active in at least 1, 2, 3, 4 or 5 of the tissues outside of the CNS described above in IV delivery.

In some embodiments, the CRE, minimal/proximal promoter or synthetic promoter of the present invention may be active in the CNS and in the peripheral nervous system (PNS). If the CRE, minimal/proximal promoter or synthetic promoter of the present invention are active in the CNS and PNS, the CRE, minimal/proximal promoter or synthetic promoter of the present invention may be called nervous system-specific (NS-specific). The PNS refers to the parts of the nervous system which are outside the brain and spinal cord. Non-limiting examples of peripheral nervous system include cranial nerves, brachial plexus, thoracoabdominal nerves, lumbar plexus, sacral plexus and neuromuscular junctions. In some embodiments, it may be desirable that the CRE, CRM, minimal/proximal promoter or promoter of the present invention shows widespread activity in the PNS. In some embodiments, the CRE, CRM, minimal/proximal promoter or synthetic promoter of the present invention is active in 1, 2, 3, 4, 5, or 6, of the areas of the PNS recited above. In some embodiments, the CRE, CRM, minimal/proximal promoter or syntenic promoter of the present invention is active in the majority of the areas in the PNS, i.e. at least 4, at least 5, or all 6 of the 6 areas of the PNS recited above. In some embodiments, synthetic promoters CNS-5 and CNS-5_v2 are active in the CNS and in the majority of the areas in the PNS, i.e. at least 4, at least 5, or all 6 of the 6 areas of the PNS recited above. In some embodiments, synthetic promoters CNS-2, CNS-3 and CNS-4 are active in the CNS and at least 1 of the areas of the PNS recited above. In some embodiments, synthetic promoters CNS-2, CNS-3 and CNS-4 are active in the CNS and in PNS sympathetic neurones.

A CNS-specific promoter can be expressed in other non-CNS cells. However, it has higher degree of expression in CNS cells such as neuronal cells in the brain and spinal cord as well as non-neuronal cells or neuronal supporting cells located in the brain and spinal cord. For example, a CNS-specific promoter expresses a gene at least 25%, or at least 35%, or at least 45%, or at least 55%, or at least 65%, or at least 75%, or at least 80%, or at least 90%, or at least 95%, or any integer between 25%-95% higher in cells located in the CNS, including neuronal and non-neuronal cells located in the brain and spinal cord as compared to cells located outside the CNS.

Expression driven by a promoter of the present invention in a desired tissue or cell may be for a period of at least 1 hour, 2, hours, 3 hours, 4 hours, 5 hours, 6 hours, 7 hours, 8 hours, 9 hours, 10 hours, 1 1 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1- 3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years.

In a further aspect of the invention, there is provided an expression cassette comprising a synthetic CNS-specific promoter of any aspect of the present invention operably linked to a sequence encoding an expression product. Suitably, the expression product is a gene, e.g. a transgene. In some embodiments, the expression product is a therapeutic expression product.

In a further aspect, there is provided a vector comprising a synthetic CNS-specific promoter or an expression cassette according to the present invention. In some embodiments, the vector is an expression vector. In some embodiments the vector is a viral vector. In some embodiments, the vector is a gene therapy vector, suitably an AAV vector, an adenoviral vector, a retroviral vector, a herpes simplex vector or a lentiviral vector. Lentiviral vectors have been extensively used as a gene transfer tool in the CNS and are known to be able to successfully transduce neurones, astrocytes and oligodendrocytes (Jakobsson and Lundberg, 2006). They are beneficial as they have relatively large cloning capacity and because viral genes are not expressed. A particularly preferred lentiviral vector system is based on HIV-1 (Jakobsson and Lundberg, 2006). Herpes simplex viral vectors and adenoviral vectors also show potential for use in as a gene transfer tool in CNS as they show successful transduction of CNS cells but are less preferred as due to their toxicity.

AAV vectors have been extensively discussed in the art. AAV vectors are of particular interest as AAV vectors do not typically integrate into the genome and do not elicit immune response. AAV serotypes 1, 2, 4, 5, 8, 9, rh10, DJ8 and 2g9 (AAV1, AAV2, AAV4, AAV5, AAV8, AAV9, AAVrh10, AAVDJ8 and AAV2g9) have been noted to achieve efficient transduction in the CNS. Therefore, AAV1, AAV2, AAV4, AAV5, AAV8, AAV9, AAVrh10, AAVVDJ8, AAV2g9 and derivatives thereof are particularly preferred AAV serotypes. In some embodiments, AAV9 is particularly preferred AAV vector. In other embodiments, AAV2g9 is a particularly preferred AAV vector (WO2014/144229). In yet other embodiments, a particularly preferred AAV vector is AAVDJ8. In some embodiments, AAVrh10 is particularly preferred AAV vector. Suitably an AAV vector comprises a viral genome which comprises a nucleic acid sequence of the present invention positioned between two inverted terminal repeats (ITRs). WO2019/028306, for example discloses various wild type and modified AAV vectors that can be used in the CNS. In one embodiment, the AAV vector is capable of penetrating the blood brain barrier following delivery of the AAV vector. In one embodiment, AAV vectors of the present invention are recombinant AAV viral vectors which are replication defective, lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ or an organism. Suitably AAV vectors for use herein comprise a virus that has been reduced to the minimum components necessary for transduction of a nucleic acid payload or cargo of interest. In this manner, AAV vectors are engineered as vehicles for specific delivery while lacking the deleterious replication and/or integration features found in wild-type viruses. In one embodiment, the AAV particle of the present invention is an scAAV. In another embodiment, the AAV particle of the present invention is an ssAAV. Methods for producing and/or modifying AAV particles are disclosed extensively in the art (see e.g. WO2000/28004; WO2001/23001; WO2004/112727; WO 2005/005610 and WO 2005/072364, which are incorporated herein by reference). In one embodiment the AAV vector comprises a capsid that allows for blood brain barrier penetration following intravascular (e.g. intravenous or intraarterial) administration (see e.g. WO2014/144229, which discusses, for example, capsids engineered for efficient crossing of the blood brain barrier, e.g. capsids or peptide inserts including VOY101, VOY201, AAVPHP.N, AAVPHP.A, AAVPHP.B, PHP.B2, PHP.B3, G2A3, G2B4, G2B5, PHP.S, and variants thereof).

Methods of making AAV vectors are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118 , 8,137,948; or International Publication Nos. WO1996039530, WO1998010088, WO 1999014354, WO1999/015685, WO1999/047691, WO2000/055342, WO2000/075353 and WO2001/023597; Methods In Molecular Biology, ed. Richard, Humana Press, N.J. (1995); O′Reilly et al, Baculovirus Expression Vectors, A Laboratory Manual, Oxford Univ. Press (1994); Samulski et al., J Fir.63:3822-8 (1989); Kajigaya et al, Proc. Nat'l. Acad. Sci. USA 88: 4646-50 (1991); Ruffing et al., J. Vir. 66:6922-30 (1992); Kimbauer et al, Vir., 219:37-44 (1996); Zhao et al, Vir.272: 382-93 (2000); the contents of each of which are herein incorporated by reference. Viral replication cells commonly used for production of recombinant AAV viral particles include but are not limited to HEK293 cells, COS cells, HeLa cells, KB cells, and other mammalian cell lines.

In some embodiments the vector is a non-viral vector, for example using cationic polymers or cationic lipids, as is known in the art. Various non-viral vectors are discussed in Selene Ingusci et al. (Gene Therapy Tools for Brain Diseases. Front. Pharmacol. 10:724. doi: 10.3389)

In a further aspect, there is provided a virion (viral particle) comprising a vector, suitably a viral vector, according to the present invention. In some embodiments the virion is an AAV virion.

In a further aspect, there is provided a pharmaceutical composition comprising a synthetic CNS-specific promoter, expression cassette, vector or virion according to the present invention.

For example, AAV vector particles may be prepared as pharmaceutical compositions. It will be understood that such compositions necessarily comprise one or more active ingredients and, most often, a pharmaceutically acceptable excipient.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In a further aspect, there is provided a synthetic CNS-specific promoter, expression cassette, vector, virion or pharmaceutical composition according to the present invention for use as medicament.

In a further aspect, there is provided a synthetic CNS-specific promoter, expression cassette, vector, virion or pharmaceutical composition according to the present invention for use in therapy, i.e. the prevention or treatment of a medical condition or disease.

Suitably the medical condition or disease is associated with aberrant gene expression, optionally aberrant gene expression in the CNS tissue or cells. Suitably the use is for gene therapy, preferably for use in the treatment of a disease involving aberrant gene expression. Suitably, the medical condition or disease involving aberrant gene expression may be a disease of the CNS. Suitably, the medical condition or disease may be a single gene disorder of the CNS. Suitably the gene therapy involves expression of a therapeutic expression product in CNS cells or tissue. Exemplary medical conditions or diseases relevant to the present aspect are discussed below.

In a further aspect, there is provided a cell comprising a synthetic CNS-specific promoter, expression cassette, vector, or virion of the present invention. In some embodiments the cell is a mammalian cell, optionally a human cell. Suitably, the cell is a CNS cell. Suitably the cell may be a neurone, an astrocyte, an oligodendrocyte, ependymal cell or a microglial cell. Suitably the cell may be a human neurone, astrocyte, oligodendrocyte, ependymal cell or microglial cell. The synthetic CNS-specific promoter can be episomal or can be in the genome of the cell.

In a further aspect, there is provided a synthetic CNS-specific CRE, synthetic CNS-specific promoter, expression cassette, vector, virion or pharmaceutical composition as described herein for use in the manufacture of a pharmaceutical composition for the treatment of a medical condition or disease. Exemplary medical conditions or diseases relevant to the present aspect are discussed below.

In a further aspect, there is provided a method for producing an expression product, the method comprising providing a synthetic CNS-specific expression cassette, vector or a virion of the present invention in CNS cells or tissue and expressing the gene of interest present in the synthetic CNS-specific expression cassette, vector or virion. The method can be in vitro or ex vivo, or it can be in vivo.

In a further aspect, there is provided a method of expressing a therapeutic transgene in a CNS cell, the method comprising introducing into the CNS cell a synthetic CNS-specific expression cassette, vector or virion as described herein and expressing the expression product (e.g. gene of interest) present in the synthetic CNS-specific expression cassette, vector or virion. The CNS cell may be, for example, a neurone, an astrocyte, an oligodendrocyte, an ependymal cell or a microglial cell.

In a further aspect, there is provided a method of therapy of a subject, preferably a human in need thereof, the method comprising:
administering to the subject an expression cassette, vector, virion or a pharmaceutical composition as described herein, which comprises a sequence encoding a therapeutic product operably linked to a promoter according to the present invention; and
expressing a therapeutic amount of the therapeutic product in the CNS of said subject.

Suitably the method is for the treatment, prophylaxis, palliation or amelioration of neurological diseases and/or disorders. Exemplary medical conditions or diseases relevant to the present aspect are discussed below.

Suitable methods of administration may be enteral (e.g. oral, sublingual, and rectal) or parenteral (e.g. injection) including intravenous, intraarterial, intracranial, intramuscular, subcutaneous, intra-articular, intrathecal, and intradermal injections. Preferred administration methods are intravenous, intraarterial, intracranial and intrathecal injection.

In some embodiments the method comprises introducing into the CNS of the subject an expression cassette, vector, virion or a pharmaceutical composition as described herein, which comprises a gene encoding a therapeutic product. A particular difficulty with introducing an expression cassette, vector, virion or a pharmaceutical composition in the CNS is the blood brain barrier. The blood brain barrier is a semipermeable border of endothelial cells that prevents certain chemicals and molecules in the bloodstream from crossing into the extracellular fluid of the central nervous system. In animal studies, this obstacle has been overcome by injection directly into the brain of the animal, such as intracranial injection, suitably intracerebroventricular (ICV) injection (see e.g. Keiser et al., Curr Protoc Mouse Biol. 2018 Dec;8(4):e57). This method of administration can be disadvantageous for gene therapy in humans as it is difficult to perform and can be dangerous for the subject.

Instead, in a gene therapy setting in human, it is preferred that the expression cassette as described herein is introduced into the CNS by intravenous or intraarterial (e.g. intra-carotid) administration of a viral vector comprising the expression cassette. Suitably, the viral vector is an AAV vector. Intravenous or intraarterial administration of some serotypes of AAV allows penetration of the AAV vectors into the brain. Minimal expression in non-CNS tissues and cells is expected due to the CNS-specificity of the synthetic CNS-specific promoters according to the present invention. Furthermore, it is expected that with the development of improved AAV capsids for CNS-penetration, penetration of AAV vectors will be improved. Intravenous or intraarterial administration is safer and less invasive than intracranial administration, while still allowing penetration through the blood brain barrier.

Suitably, the medical condition or disease is a medical condition or disease of the CNS, e.g. a neurological disease and/or disorder. Suitably, the medical condition or disease may be selected from, for example: dopamine transporter deficiency syndrome, an attention deficit/hyperactivity disorder (ADHD), bipolar disorder, epilepsy, multiple sclerosis, tauopathies, Alzheimer's disease, Huntington's disease, Parkinson's disease, Krabbe's disease, adrenoleukodystrophy, motor neurone disease, cerebral palsy, Batten disease, Gaucher disease, Tay Sachs disease, Rett syndrome, Sandhoff disease, Charcot-Marie-Tooth disease, Angelman syndrome, Canavan disease, Late infantile neuronal ceroid lipofuscinosis, Mucopolysaccharidosis IIIA, Mucopolysaccharidosis IIIB, Metachromatic leukodystrophy, heritable lysosomal storage diseases such as Niemann-Pick disease type C1, and/or neuronal ceroid lipofuscinoses such as Batten disease, progressive supranuclear palsy, corticobasal syndrome, and brain cancer (including astrocytomas and glioblastomas).

Suitably, the nucleic acid encoding an expression product may be one of the genes selected from the group consisting of: NPC1, EAAT2, NPY, CYP46A1, GLB1, APOE (or APOE2), HEX, CLN1, CLN2, CLN3, CLN4, CLN5, CLN6, SUMF1, DCTN1, PRPH, SOD1, NEFH, GBA, IDUA, NAGLU, GUSB, ARSA, MANB, AADC, GDNF, NTN, ASP, MECP2, PTCHD1, GJB1, UBE3A, HEXA, FXN and MOG.

Additionally, or alternatively, the expression product may be an antibody, antibody fragment or antibody like scaffold protein.

Additionally, or alternatively, the expression product may be may a gene editing system (such as a CRISPR-Cas9 system, TALEN, ZFN, etc.) directed to the disease allele.

Additionally, or alternatively, the expression product may be one or more modulatory polynucleotides, e.g., RNA or DNA molecules as therapeutic agents. For example the modulatory polynucleotide may be a miRNA or siRNA. Target genes may be any of the genes associated with any neurological disease such as, but not limited to, those listed herein. For example, siRNA duplexes or encoded dsRNA can reduce or silence target gene expression in CNS cells, thereby ameliorating symptoms of neurological disease. In one non-limiting example, the target gene is huntingtin (HTT). In another non-limiting example he target gene is microtubule-associated protein tau (MAPT).

In a further aspect, there is provided a synthetic CNS-specific promoter comprising or consisting of SEQ ID NO: 1 or SEQ ID NO: 21. Suitably, the synthetic CNS-specific promoter is able to promote widespread intracranial expression of an expression product operably linked to the CNS-specific promoter when it is administered via ICV injection. Suitably, the synthetic CNS-specific promoter is active in at least 6 areas of the brain. Suitably, when the synthetic CNS-specific promoter is administered via ICV injection, the synthetic CNS-specific promoter is able to promote CNS-specific expression of an expression product at a level at least 100%, 150% or 200% compared to Synapsin-1 (SEQ ID NO: 14) in the brain. Suitably, the synthetic CNS-specific promoter is able to promote expression in cortex and the hippocampus when it is administered via ICV injection.

In a further aspect, there is provided a method of expressing an expression product in the CNS, the method comprising introducing into the CNS cell an expression cassette comprising a synthetic CNS-specific promoter comprising or consisting of SEQ ID NO: 1 or SEQ ID NO: 21 operably linked to the expression product. Suitably, the expression cassette is introduced into the CNS via ICV injection and the expression product is widespread in the brain. Suitably, the expression of the expression product in the brain is in at least 6 areas of the brain. Suitably, the synthetic CNS-specific promoter is able to promote CNS-specific expression of an expression product at a level at least 100%, 150% or 200% compared to Synapsin-1 (SEQ ID NO: 14) in the brain. Suitably, the expression cassette is introduced into the CNS via ICV injection and the expression product is expressed in the cortex and the hippocampus.

In a further aspect, there is provided a synthetic CNS-specific promoter comprising or consisting of SEQ ID NO: 2, SEQ ID NO: 25 or SEQ ID NO: 7, or functional variants thereof as discussed above. Suitably, such a synthetic CNS-specific promoter is able to promote widespread expression in the brain of an expression product from a nucleic acid operably linked to the CNS-specific promoter when administered via ICV injection. Suitably, the synthetic CNS-specific promoter is active in at least 6 areas of the brain. Suitably, the synthetic CNS-specific promoter comprising or consisting of SEQ ID NO: 2, or a functional variant thereof, is able to promote widespread intracranial expression of an expression product operably linked to the CNS-specific promoter when administered via IV injection. Suitably, the synthetic CNS-specific promoter comprising or consisting of SEQ ID NO: 2, or a functional variant thereof, does not promoter expression in the midbrain. Suitably, the synthetic CNS-specific promoter comprising or consisting of SEQ ID NO: 7 or SEQ ID NO: 25, or a functional variant thereof, is able to promote expression in the cortex, hippocampus and midbrain of an expression product operably linked to the CNS-specific promoter when administered via IV injection.

In a further aspect, there is provided a method of expressing an expression product in the CNS, the method comprising introducing into the CNS cell an expression cassette comprising a synthetic CNS-specific promoter comprising or consisting of SEQ ID NO: 2, or a functional variant thereof, SEQ ID NO: 25, or a functional variant thereof, or SEQ ID NO: 7, or a functional variant thereof, operably linked to a nucleic acid encoding the expression product. Suitably, the expression cassette is introduced into the CNS via ICV injection and the expression of the expression product is widespread in the brain. Suitably, the expression of the expression product in the brain is in at least 6 areas of the brain as discussed above. Suitably, the expression cassette comprising or consisting of SEQ ID NO: 2, or a functional variant thereof, is introduced into the CNS via IV injection and the expression of the expression product is widespread in the brain, but not in the midbrain. Suitably, the expression cassette comprising or consisting of SEQ ID NO: 7 or SEQ ID NO: 25, or a functional variant thereof, is introduced into the CNS via IV injection and the expression of the expression product is expressed in the cortex, hippocampus and midbrain, but not in the midbrain.

In a further aspect, there is provided a synthetic CNS-specific promoter comprising or consisting of SEQ ID NO: 3, SEQ ID NO: 22 or SEQ ID NO: 4, or functional variants thereof as discussed above. Suitably, the synthetic CNS-specific promoter is able to promote expression in the cortex and hippocampus when administered via ICV injection. Suitably, the synthetic CNS-specific promoter is not active or is minimally active in the other areas of the brain. Suitably, the synthetic CNS-specific promoter comprising or consisting of SEQ ID NO: 3, or a functional variant thereof, SEQ ID NO: 22, or a functional variant thereof, or SEQ ID NO: 4, or a functional variant thereof, is able to promote expression in the cortex, striatum and hippocampus when administered via IV injection. Suitably, the synthetic CNS-specific promoter comprising or consisting of SEQ ID NO: 4 or SEQ ID NO: 22, or a functional variant thereof, is able to additionally promote expression in the midbrain.

In a further aspect, there is provided a method of expressing an expression product in the CNS, the method comprising introducing into the CNS cell an expression cassette comprising a synthetic CNS-specific promoter comprising or consisting of SEQ ID NO: 3, or a functional variant thereof, SEQ ID NO: 22, or a functional variant thereof, or SEQ ID NO: 4, or a functional variant thereof, operably linked to a nucleic acid encoding the expression product. Suitably, the expression cassette is introduced into the CNS via ICV injection and the expression product is expressed in the cortex and hippocampus. Suitably, the expression of the expression is minimal in the other areas of the brain. Suitably, the expression cassette is introduced into the CNS via ICV injection and the expression of the expression product is expressed in the cortex and hippocampus. Suitably, the expression cassette is introduced into the CNS via IV injection and the expression of the expression product is expressed in the cortex, striatum and hippocampus.

In a further aspect, there is provided a synthetic CNS-specific promoter comprising or consisting of SEQ ID NO: 5 or SEQ ID NO: 23, or a functional variant thereof as discussed above. Suitably, the synthetic CNS-specific promoter is able to promote expression in the cortex, striatum, hippocampus and midbrain. Suitably, the synthetic CNS-specific promoter is not active or is minimally active in the other areas of the brain. Suitably, the synthetic CNS-specific promoter is administered via ICV injection.

In a further aspect, there is provided a method of expressing an expression product in the CNS, the method comprising introducing into the CNS cell an expression cassette comprising a synthetic CNS-specific promoter comprising or consisting of SEQ ID NO: 5 or SEQ ID NO: 23, or a functional variant thereof, operably linked to a nucleic acid encoding the expression product. Suitably, the expression cassette is introduced into the CNS via ICV injection. Suitably, the expression product is expressed in the cortex, striatum, hippocampus and midbrain. Suitably, the expression is minimal in the other areas of the brain.

In a further aspect, there is provided a synthetic CNS-specific promoter comprising or consisting of SEQ ID NO: 6, SEQ ID NO: 24, SEQ ID NO: 26 or SEQ ID NO: 8, or functional variant thereof as discussed above. Suitably, the synthetic CNS-specific promoter is able to promote expression in the hippocampus, cortex and the midbrain when administered via ICV injection. Suitably, the synthetic CNS-specific promoter comprising or consisting of SEQ ID NO: 6 or SEQ ID NO: 24, or a functional variant thereof, is able to promote expression in the hippocampus, midbrain and cerebellum when administered via IV injection. Suitably, the synthetic CNS-specific promoter comprising or consisting of SEQ ID NO: 8 or SEQ ID NO: 26, or a functional variant thereof, is able to promote expression in the hippocampus and the midbrain when administered via IV injection. Suitably, the synthetic CNS-specific promoter is not active or is minimally active in the other areas of the brain. Suitably, the synthetic CNS-specific promoter comprising or consisting of SEQ ID NO: 6, or a functional variant thereof, SEQ ID NO: 24, or a functional variant thereof, SEQ ID NO: 26, or a functional variant thereof, or SEQ ID NO: 8, or a functional variant thereof, is primarily active in neurones. Suitably, the synthetic CNS-specific promoter comprising or consisting of SEQ ID NO: 8 or SEQ ID NO: 26, or a functional variant thereof, is primarily active in dopaminergic neurones.

In a further aspect, there is provided a method of expressing an expression product in the CNS, the method comprising introducing into the CNS cell an expression cassette comprising a synthetic CNS-specific promoter comprising or consisting of SEQ ID NO: 6, SEQ ID NO: 24, SEQ ID NO: 26 or SEQ ID NO:8 operably linked to a nucleic acid encoding the expression product. Suitably, the expression cassette is introduced into the CNS via ICV injection and the expression product is expressed in the hippocampus, cortex and the midbrain. Suitably, the expression cassette comprising or consisting of SEQ ID NO: 6 or SEQ ID NO: 24, or a functional variant thereof, is introduced into the CNS via IV injection and the expression product is expressed in the hippocampus, midbrain and cerebellum. Suitably, the expression cassette comprising or consisting of SEQ ID NO: 8 or SEQ ID NO: 26, or a functional variant thereof, is introduced into the CNS via IV injection and the expression product is expressed in the hippocampus and the midbrain. Suitably, the expression of the expression is minimal in the other areas of the brain.

In a further aspect, there is provided a method of expressing an expression product in a dopaminergic neurone, the method comprising introducing into the dopaminergic neurone a synthetic CNS-specific expression cassette via IV injection, wherein the CNS-specific expression cassette comprises SEQ ID NO: 8 or SEQ ID NO: 26, or a functional variant thereof.

### BRIEF DESCRIPTION OF THE FIGURES

FIG. 1A shows the expression pattern of the DAT1/SLC6A3 gene in a coronal section from an adult mouse brain (taken from the Alan Mouse brain atlas; mouse.brain-map.org). DAT1/SLC6A3 is highly expressed in the midbrain.

FIG. 1B shows the expression pattern of the UBE3A gene in a coronal section from an adult mouse brain (taken from the Alan Mouse brain atlas; mouse.brain-map.org). UBE3A is widely expressed in the brain.

FIG. 2A shows the intracranial biodistribution in sagittal sections of the transgene GFP under the control of CNS-1 (SEQ ID NO: 1), CNS-2 (SEQ ID NO: 2), CNS-3 (SEQ ID NO: 3) and CNS-4 (SEQ ID NO: 4) and the control promoter hSyn1 delivered by ICV and IV. Scale bar is 1 mm.

FIG. 2B shows the intracranial biodistribution in sagittal sections of the transgene GFP under the control of CNS-5 (SEQ ID NO: 23), CNS-6 (SEQ ID NO: 24), CNS-7(SEQ ID NO: 25) and CNS-8 (SEQ ID NO: 26) delivered by ICV and IV. Scale bar is 1 mm.

FIG. 3A shows the intracranial biodistribution in coronal sections of the transgene GFP under the control of CNS-1 (SEQ ID NO: 1), CNS-2 (SEQ ID NO: 2), CNS-3 (SEQ ID NO: 3) and CNS-4 (SEQ ID NO: 4) delivered by ICV. Scale bar is 1 mm.

FIG. 3B shows the intracranial biodistribution in coronal sections of the transgene GFP under the control of CNS-5 (SEQ ID NO: 23), CNS-6 (SEQ ID NO: 24), CNS-7 (SEQ ID NO: 25) and CNS-8 (SEQ ID NO: 26) and the control promoter hSyn1 delivered by ICV. Scale bar is 1 mm.

FIG. 4A shows the intracranial biodistribution in coronal sections of the transgene GFP under the control of CNS-1 (SEQ ID NO: 1), CNS-2 (SEQ ID NO: 2), CNS-3 (SEQ ID NO: 3) and CNS-4 (SEQ ID NO: 4) delivered by IV. Scale bar is 1 mm.

FIG. 4B shows the intracranial biodistribution in coronal sections of the transgene GFP under the control of CNS-5 (SEQ ID NO: 23), CNS-6 (SEQ ID NO: 24), CNS-7 (SEQ ID NO: 25) and CNS-8 (SEQ ID NO: 26) delivered by IV. Scale bar is 1 mm.

FIG. 5A shows the intracranial biodistribution at higher magnification, and in different parts of the brain, of the transgene GFP under the control of CNS-1 (SEQ ID NO: 1) , CNS-2 (SEQ ID NO: 2), CNS-3 (SEQ ID NO: 3) and CNS-4 (SEQ ID NO: 4) and the control promoter hSyn1 delivered by ICV. Scale bar is 100 μm.

FIG. 5B shows the intracranial biodistribution at higher magnification, and in different parts of the brain, of the transgene GFP under the control of CNS-5 (SEQ ID NO: 23), CNS-6 (SEQ ID NO: 24), CNS-7 (SEQ ID NO: 25) and CNS-8 (SEQ ID NO: 26) and the control promoter hSyn1 delivered by ICV. Scale bar is 100 μm.

FIG. 6A shows the intracranial biodistribution at higher magnification, and in different parts of the brain, of the transgene GFP under the control of CNS-1 (SEQ ID NO: 1), CNS-2 (SEQ ID NO: 2), CNS-3 (SEQ ID NO: 3) and CNS-4 (SEQ ID NO: 4) delivered by IV as well as Uninjected control. Scale bar is 100 μm.

FIG. 6B shows the intracranial biodistribution brain at higher magnification, and in different parts of the brain, of the transgene GFP under the control of CNS-5 (SEQ ID NO: 23), CNS-6 (SEQ ID NO: 24), CNS-7 (SEQ ID NO: 25) and CNS-8 (SEQ ID NO: 26) delivered by IV as well as uninjected control. Scale bar is 100 μm.

FIG. 7A shows the biodistribution in the midbrain of the transgene GFP under the control of CNS-1 (SEQ ID NO: 1), CNS-2 (SEQ ID NO: 2), CNS-3 (SEQ ID NO: 3) and CNS-4 (SEQ ID NO: 4) and the control promoter hSyn1 delivered by ICV. Left column shows GFP expression, middle column shows TH+ positive cells (dopaminergic neurones) and the right column shows an overlay of the two together with the nuclear dye DAPI. Scale bar is 25 μm.

FIG. 7B shows the biodistribution in the midbrain of the transgene GFP under the control of CNS-5 (SEQ ID NO: 23), CNS-6 (SEQ ID NO: 24), CNS-7 (SEQ ID NO: 25) and CNS-8 (SEQ ID NO: 26) delivered by ICV. Left column shows GFP expression, middle column shows TH+ positive cells (dopaminergic neurones) and the right column shows an overlay of the two together with the nuclear dye DAPI. Scale bar is 25 μm.

FIG. 8A shows the biodistribution in the midbrain of the transgene GFP under the control of CNS-1 (SEQ ID NO: 1), CNS-2 (SEQ ID NO: 2), CNS-3 (SEQ ID NO: 3) and CNS-4 (SEQ ID NO: 4) delivered by IV as well as uninjected control. Left column shows GFP expression, middle column shows TH+ positive cells (dopaminergic neurones) and the right column shows an overlay of the two together with the nuclear dye DAPI. Scale bar is 25 μm.

FIG. 8B shows the biodistribution in the midbrain of the transgene GFP under the control of CNS-5 (SEQ ID NO: 23), CNS-6 (SEQ ID NO: 24), CNS-7 (SEQ ID NO: 25) and CNS-8 (SEQ ID NO: 26) delivered by IV. Left column shows GFP expression, middle column shows TH+positive cells (dopaminergic neurones) and the right column shows an overlay of the two together with the nuclear dye DAPI. Scale bar is 25 μm.

FIG. 9 shows biodistribution in different tissues of the transgene GFP under the control of CNS-1 — 8 (SEQ ID NO: 1-4, 23-26) and the control promoter Synapsin1 (SEQ ID NO: 14) delivered by ICV or IV. For this data, RNA extracted from systemic organs was converted into RNA and quantified by qPCR. Different promoters out of CNS-1-8 (SEQ ID NOs:1-4,23-26) showed off-target expression in liver, kidney, heart, skeletal muscle or spleen.

FIG. 10 shows percentage GFP immunoreactivity in different brain regions following ICV or IV delivery of GFP driven by CNS 1-8 (SEQ ID NOs: 1-4,23-26) or Synapsin-1 (SEQ ID NO: 14). The data was obtained by quantitative measurement of 10 non-overlapping RGB images of GFP staining intensity by thresholding analysis in cortex, hippocampus, striatum, midbrain and cerebellum (mean±SEM). Images were taken at ×40 magnification through discrete brain regions keeping constant settings. The foreground immunostaining was defined by averaging of the highest and lowest signals. Data is represented as the mean percentage area of immunoreactivity per field for each region of interest (n=3). With ICV delivery, expression is highest in cortex and hippocampal brain regions. CNS 1-8 (SEQ ID NO: 1-4, 23-26) show higher expression in the hippocampus than hSyn1 control. CNS-1 (SEQ ID NO: 1) shows higher expression in hippocampus, midbrain and cerebellum compared to hSyn1 with ICV delivery.

FIG. 11 shows the expression of GFP under the control of CNS-1 (SEQ ID NO: 1) in ICV delivery. Magnification is ×40. NeuN is a marker for neuronal nuclei. GFAP is a marker for astrocytes and IBA1 is a marker for microglia. GFP expression driven by the CNS-1 (SEQ ID NO: 1) promoter is primarily neuronal.

FIG. 12 shows the intracranial biodistribution in sagittal sections of the transgene GFP under the control of CNS-8 (SEQ ID NO: 26) and the control promoter hSyn1 delivered by ICV and IV. Scale bar is 1 mm.

FIG. 13A shows the intracranial biodistribution in coronal sections of the transgene GFP under the control of CNS-8 (SEQ ID NO: 26) and the control promoter hSyn1 delivered by ICV. On the left-hand side, the scale bar is 1 mm. On the right-hand side, the areas of the brain are shown at higher magnification. Scale bar is 100 μm.

FIG. 13B shows the intracranial biodistribution in coronal sections of the transgene GFP under the control of CNS-8 (SEQ ID NO: 26) and the control promoter hSyn1 delivered by IV. On the left-hand side, the scale bar is 1 mm. On the right-hand side, the areas of the brain are shown at higher magnification. Scale bar is 100 μm.

FIG. 14A shows the biodistribution in the midbrain of the transgene GFP under the control of CNS-8 (SEQ ID NO: 26) delivered by ICV (top) and IV (bottom). Left column shows TH+ positive cells (dopaminergic neurones), middle column shows GFP expression and the right column shows an overlay of the two together with the nuclear dye DAPI. Scale bar is 25 μm.

FIG. 14B show quantification of the percentage of dopaminergic neurones showing GFP expression (TH+ GFP+ cells) out of all dopaminergic neurones. The left-hand part of the graph shows percentage of dopaminergic neurones showing GFP under the control of the control promoter Syn-1 in ICV and IV delivery. The middle part of the graph shows quantification of the percentage of dopaminergic neurones showing GFP expression under the control of CNS-8 (SEQ ID NO: 26) when low dose was administered (Example 1) in ICV and IV delivery. The right-hand part of the graph shows the percentage of dopaminergic neurones showing GFP expression under the control of CNS-8 (SEQ ID NO: 26) when the high dose was administered (Example 2) in ICV and IV delivery.

FIG. 15 shows a comparison of the biodistribution in different tissues of the transgene GFP under the control of CNS-8 (SEQ ID NO: 26) when a low or a high dose was administered. The left-hand side shows biodistribution of GFP under the control of CNS-8 (SEQ ID NO: 26) when a low dose was administered, and the right-hand side shows biodistribution of GFP under the control of CNS-8 (SEQ ID NO: 26) when a high dose was administered. The data for biodistribution of GFP under the control of CNS-8 (SEQ ID NO: 26) when a low dose was administered is the same as the data shown in FIG. 9. For this data, RNA extracted from systemic organs was converted into RNA and quantified by qPCR.

FIG. 16A shows the expression pattern of the faf1 gene in mouse PNS neurones from single cell transcriptomic data (Zeisel et al., 2018). Dark grey denotes high expression, white denotes no expression and light grey denotes low expression. faf1 is expressed in many PNS neurones.

FIG. 16B shows the expression pattern of the pitx3 gene in PNS neurones from single cell transcriptomic data (Zeisel et al., 2018). Dark grey denotes high expression, white denotes no expression and light grey denotes low expression. pixt3 is expressed in sympathetic PNS neurones.

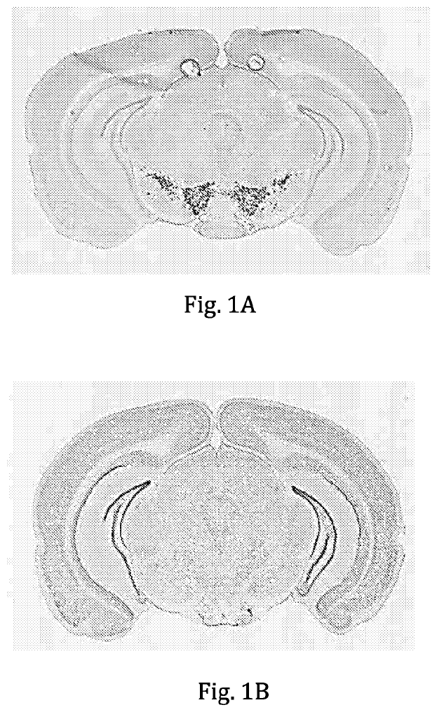

### DETAILED DESCRIPTION OF EMBODIMENTS OF THE INVENTION AND EXAMPLES

CREs and Functional Variants Thereof

Disclosed herein are various CREs that can be used in construction of CNS-specific promoters. Suitably, the CREs are CNS-specific. These CREs are generally derived from genomic promoter and enhancer sequences, but they are used herein in contexts quite different from their native genomic environment. Generally, the CREs constitute small parts of much larger genomic regulatory domains, which control expression of the genes with which they are normally associated. It has been surprisingly found that these CREs, many of which are very small, can be isolated form their normal environment and retain CNS-specific regulatory activity. This is surprising because the removal of a regulatory sequence from the complex and “three dimensional” natural context in the genome often results in a significant loss of activity, so there is no reason to expect a given CRE to retain the levels of activity observed once removed from their natural environment. It is even more surprising when a CRE retain CNS-specific activity in an AAV vector. This is particularly the case as an AAV vector comprises Inverted Terminal Repeat (ITR) and has a different DNA structure compared to the genome and both ITRs and the DNA structure are known to influence the activity of CREs.

It should be noted that the sequences of the CREs of the present invention can be altered without causing a substantial loss of activity. Functional variants of the CREs can be prepared by modifying the sequence of the CREs, provided that modifications which are significantly detrimental to activity of the CRE are avoided. In view of the information provided in the present disclosure, modification of CREs to provide functional variants is straightforward. Moreover, the present disclosure provides methodologies for simply assessing the functionality of any given CRE variant.

The relatively small size of certain CREs according to the present invention is advantageous because it allows for the CREs, more specifically promoters containing them, to be provided in vectors while taking up the minimal amount of the payload of the vector. This is particularly important when a CRE is used in a vector with limited capacity, such as an AAV-based vector.

CREs of the present invention comprise certain CNS-specific TFBS. It is generally desired that in functional variants of the CREs these CNS-specific TFBS remain functional. The skilled person is well aware that TFBS sequences can vary yet retain functionality. In view of this, the sequence for a TFBS is typically illustrated by a consensus sequence from which some degree of variation is typically present. Further information about the variation that occurs in a TFBS can be illustrated using a positional weight matrix (PWM), which represents the frequency with which a given nucleotide is typically found at a given location in the consensus sequence. Details of TF consensus sequences and associated positional weight matrices can be found in, for example, the Jaspar or Transfac databases http://jaspar.genereg.net/ and http://gene-regulation.com/pub/databases.html). This information allows the skilled person to modify the sequence in any given TFBS of a CRE in a manner which retains, and in some cases even increases, CRE functionality. In view of this the skilled person has ample guidance on how the TFBS for any given TF can be modified, while maintaining ability to bind the desired TF; the Jaspar system will, for example, score a putative TFBS based on its similarity to a given PWM. Furthermore, CREs can be scanned against all PWM from JASPAR database to identify/analyse all TFBS. The skilled person can of course find additional guidance in the literature, and, moreover, routine experimentation can be used to confirm TF binding to a putative TFBS in any variant CRE.

It will be apparent that significant sequence modification in a CRE, even within TFBS in a CRE, can be made while retaining function.

CREs of the present invention can be used in combination with a wide range of suitable minimal promoters or CNS-specific proximal promoters.

Functional variants of a CRE include sequences which vary from the reference CRE element, but which substantially retain activity as CNS-specific CREs. It will be appreciated by the skilled person that it is possible to vary the sequence of a CRE while retaining its ability to recruit suitable CNS-specific transcription factors (TFs) and thereby enhance expression. A functional variant of a CRE can comprise substitutions, deletions and/or insertions compared to a reference CRE, provided they do not render the CRE substantially non-functional.

In some embodiments, a functional variant of a CRE can be viewed as a CRE which, when substituted in place of a reference CRE in a promoter, substantially retains its activity. For example, a CNS-specific promoter which comprises a functional variant of a given CRE preferably retains at least 80% of its activity, more preferably at least 90% of its activity, more preferably at least 95% of its activity, and yet more preferably 100% of its activity (compared to the reference promoter comprising the unmodified CRE).

Suitably, functional variants of a CRE retain a significant level of sequence identity to a reference CRE. Suitably functional variants comprise a sequence that is at least 70% identical to the reference CRE, more preferably at least 80%, 90%, 95% or 99% identical to the reference CRE.

Retention of activity can be assessed by comparing expression of a suitable reporter under the control of the reference promoter with an otherwise identical promoter comprising the substituted CRE under equivalent conditions. Suitable assays for assessing CNS-specific promoter activity are disclosed herein, e.g. in the examples.

In some embodiments, a CRE can be combined with one or more additional CREs to create a cis-regulatory module (CRM). Additional CREs can be provided upstream of the CREs according to the present invention, or downstream of the according to the present invention. The additional CREs can be CREs disclosed herein, or they can be other CREs. Suitably, the additional CREs are CNS-specific.

CREs according to the present invention or CRMs comprising CREs according to the present invention may comprise one or more additional regulatory elements. For example, they may comprise an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the CRE or CRM substantially non-functional.

A comprising CREs according to the present invention may comprise spacers between the CRM and the minimal or proximal promoter and/or between CREs. Additionally, or alternatively, a spacer may be present on the 5′ end of the CRM.

It will be apparent that a CRE according to the present invention or a CRM comprising a CRE according to this invention, or functional variants thereof, can be combined with any suitable promoter elements in order to provide a synthetic CNS-specific promoter according to the present invention. Suitably, the promoter elements is CNS-specific proximal promoter.

In many instances, shorter promoter sequences are preferred, particularly for use in situations where a vector (e.g. a viral vector such as AAV) has limited capacity. Accordingly, in some embodiments the synthetic CNS specific CRM comprising at least one of the CREs according to SEQ ID NOs 9-11, 28-31 or a functional variant thereof has length of 1000 or fewer nucleotides, for example 950, 900, 850, 800, 750, 700, 650, 600, 550, 500, 450, 400, 350, 300, 250, 200, 150, 100, 75, 60, 50 or fewer nucleotides.

Synthetic CNS-Specific Promoters and Functional Variants Thereof

Various synthetic CNS-specific promoters are disclosed herein. A functional variant of a reference synthetic CNS-specific promoter is a promoter which comprises a sequence which varies from the reference synthetic CNS-specific promoter, but which substantially retains CNS-specific promoter activity. It will be appreciated by the skilled person that it is possible to vary the sequence of a synthetic CNS-specific promoter while retaining its ability to recruit suitable CNS-specific transcription factors (TFs) and to recruit RNA polymerase II to provide CNS-specific expression of an operably linked sequence (e.g. an open reading frame). A functional variant of a synthetic CNS-specific promoter can comprise substitutions, deletions and/or insertions compared to a reference promoter, provided such substitutions, deletions and/or insertions do not render the synthetic CNS-specific promoter substantially non-functional compared to the reference promoter.

Accordingly, in some embodiments, a functional variant of a synthetic CNS-specific promoter can be viewed as a variant which substantially retains the CNS-specific promoter activity of the reference promoter. For example, a functional variant of a synthetic CNS-specific promoter preferably retains at least 70% of the activity of the reference promoter, more preferably at least 80% of its activity, more preferably at least 90% of its activity, more preferably at least 95% of its activity, and yet more preferably 100% of its activity.

Functional variants of a synthetic CNS-specific promoter often retain a significant level of sequence similarity to a reference synthetic CNS-specific promoter. In some embodiments, functional variants comprise a sequence that is at least 70% identical to the reference synthetic CNS-specific promoter, more preferably at least 80%, 90%, 95% or 99% identical to the reference synthetic CNS-specific promoter.

Activity in a functional variant can be assessed by comparing expression of a suitable reporter under the control of the reference synthetic CNS-specific promoter with the putative functional variant under equivalent conditions. Suitable assays for assessing CNS-specific promoter activity are disclosed herein, e.g. in the examples.

Functional variants of a given synthetic CNS-specific promoter can comprise functional variants of a CRE present in the reference synthetic CNS-specific promoter. Functional variants of a given synthetic CNS-specific promoter can comprise functional variants of the CRE present in the reference synthetic CNS-specific promoter. Functional variants of a given synthetic CNS-specific promoter can comprise functional variants of the promoter element, or a different promoter element when compared to the reference synthetic CNS-specific promoter.

Functional variants of a given synthetic CNS-specific promoter can comprise one or more additional CREs to those present in a reference synthetic CNS-specific promoter. Additional CREs can, for example, be provided upstream of the CREs present in the reference synthetic CNS-specific promoter or downstream of the CREs present in the reference synthetic CNS-specific promoter. The additional CREs can be CREs disclosed herein, or they can be other CREs.

Functional variants of a given synthetic CNS-specific promoter can comprise additional spacers between adjacent elements (CREs, CRM or promoter element) or, if one or more spacers are present in the reference synthetic CNS-specific promoter, said one or more spacers can be longer or shorter than in the reference synthetic CNS-specific promoter.

It will be apparent that synthetic CNS-specific promoters of the present invention can comprise a CRE of the present invention or a CRM comprising a CRE of the present invention and additional regulatory sequences. For example, they may comprise one or more additional CREs, an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the promoter substantially non-functional.

In some embodiments, the CNS-specific promoters as set out above are operably linked to one or more additional regulatory sequences. An additional regulatory sequence can, for example, enhance expression compared to a CNS-specific promoter which is not operably linked the additional regulatory sequence. Generally, it is preferred that the additional regulatory sequence does not substantively reduce the specificity of a CNS-specific promoter.

For example, a CNS-specific promoter according to the present invention can be operably linked to a sequence encoding a UTR (e.g. a 5′ and/or 3′ UTR), and/or an intron, or suchlike.

In some embodiments, the CNS-specific promoter is operably linked to sequence encoding a UTR, e.g. a 5′ UTR. A 5′ UTR can contain various elements that can regulate gene expression. The 5′ UTR in a natural gene begins at the transcription start site and ends one nucleotide before the start codon of the coding region. It should be noted that 5′ UTRs as referred to herein may be an entire naturally occurring 5′ UTR or it may be a portion of a naturally occurring 5′ UTR. The 5′UTR may also be partially or entirely synthetic. In eukaryotes, 5′ UTRs have a median length of approximately 150 nucleotides, but in some cases they can be considerably longer. Regulatory sequences that can be found in 5′ UTRs include, but are not limited to:
Binding sites for proteins, that may affect the mRNA's stability or translation;
Riboswitches;
Sequences that promote or inhibit translation initiation; and
Introns within 5′ UTRs have been linked to regulation of gene expression and mRNA export.

When a regulatory sequence comprises both a 5′ UTR and an intron, it may be called 5′UTR and intron.

In some embodiments, a synthetic CNS-specific promoter as set out above is operably linked to a sequence encoding a 5′ UTR and an intron. In some embodiments, the 5′ UTR and intron is derived from the CMV major immediate gene (CMV-IE gene). For example, the 5′ UTR and intron from the CMV-IE gene suitably comprises the CMV-IE gene exon 1 and the CMV-IE gene exon 1, or portions thereof.

In some embodiments, the promoter element may be modified in view of the linkage to the 5 ‘UTR, for example sequences downstream of the transcription start site (TSS) in the promoter element can be removed (e.g. replaced with the 5’ UTR).

The CMV-IE 5′UTR and intron is described in Simari, et al., Molecular Medicine 4: 700-706, 1998 “Requirements for Enhanced Transgene Expression by Untranslated Sequences from the Human Cytomegalovirus Immediate-Early Gene”, which is incorporated herein by reference. Variants of the CMV-IE 5′ UTR and intron sequences discussed in Simari, et al. are also set out in WO2002/031137, incorporated by reference, and the regulatory sequences disclosed therein can also be used.

Other regulatory elements such as other UTRs which can be used in combination with a promoter are known in the art, e.g. in Leppek, K., Das, R. & Barna, M. “Functional 5′ UTR mRNA structures in eukaryotic translation regulation and how to find them”. Nat Rev Mol Cell Biol 19, 158-174 (2018), which is incorporated herein by reference.

In some embodiments, any one of the CNS-specific promoters described herein, or variants thereof, is linked to a sequence encoding a 5′ UTR and/or a 5′UTR and an intron.

In some embodiments the sequence encoding the 5′ UTR and intron comprises SEQ ID NO: 27, or a functional variant thereof. In some embodiments, functional variants may have a sequence that is at least 60%, 65%, 70%, 75%, 80%, 85%, 90%, 95%, 96%, 97%, 98%, or 99% identical thereto. SEQ ID NO: 27 encodes a CMV-IE 5′ UTR and intron.

Tcagatcgcctggagacgccatccacgctgttttgacctccatagaagacaccgggaccgatccagcctccgcggccgggaa cggtgcattggaacgcggattccccgtgccaagagtgacgtaagtaccgcctatagactctataggcacacccctttggctcttat gcatgaacggtggagggcagtgtagtctgagcagtactcgttgctgccgcgcgcgccaccagacataatagctgacagactaa cagactgttcctttccatgggtcttttctgcag (SEQ ID NO: 27)

In some embodiments, the CNS-specific promoter CNS-1 (SEQ ID NO:1) is operably linked to the CMV-IE 5′ UTR and intron (SEQ ID NO: 27) to provide SEQ ID NO: 21.

In some embodiments, the CNS-specific promoter CNS-4 (SEQ ID NO:4) is operably linked to the CMV-IE 5′ UTR and intron (SEQ ID NO: 27) to provide SEQ ID NO: 22.

In some embodiments, any of the CNS-specific promoter CNS-2, CNS-3, CNS-5, CNS-5_v2, CNS-6, CNS-6_v2, CNS-7, CNS-7_v2, CNS-8, CNS-8_v2 are operably linked to the CMV-IE 5′ UTR and intron (SEQ ID NO: 27).

Preferred synthetic CNS-specific promoters of the present invention exhibit CNS-specific promoter activity which is at least 15%, 20%, 25%, 30%, 40%, 50%, 60%, 70%, 80%, 90%, 100%, 125%, 150%, 175%, 200%, 250%, 300%, 350% or 400% of the activity exhibited by the Synapsin-1, Camk2a or the NSE promoter in CNS cells. In many cases higher levels of promoter activity is preferred, but this is not always the case; thus, in some cases more moderate levels of expression may be preferred. In some cases, it is desirable to have available a range of promoters of different activity levels to allow the level of expression to be tailored to requirements; the present disclose provides promoters with such a range of activities. Activity of a given synthetic CNS-specific promoter of the present invention compared to Syn-1 can be assessed by comparing CNS-specific expression of a reporter gene under control of the synthetic CNS-specific promoter with expression of the same reporter under control of the Syn-1 promoter, when the two promoters are provided in otherwise equivalent expression constructs and under equivalent conditions.

In addition to different activity levels, in some cases, it is desirable to have available a range of promoters with activity in different regions of the brain. Additionally, it may be desirable to have a range of promoters with different activity levels across different regions of the brain to allow the level of expression to be tailored to requirements; the present disclose provides promoters with such a range of activities. In some cases, expression in a specific region of the brain is desired. In some embodiments, expression in a specific region of the brain is desired with little or no expression in the rest of the brain. This may be the case, for example, in the treatment of diseases such as dopamine transporter deficiency syndrome where expression is desired in the midbrain. In some preferred embodiments, the CNS-specific promoter according to the present invention shows activity in the midbrain. In some preferred embodiments, the CNS-specific promoter according to the present invention shows activity in the midbrain with little or no activity in other areas of the brain. In some preferred embodiments, the CNS-specific promoter according to the present invention shows activity in dopaminergic neurones. In some embodiments, the CNS-specific promoter according to the present invention shows activity in dopaminergic neurones with little or no expression in other CNS cell types or CNS subtypes. Preferred synthetic CNS-specific promoters of the present invention exhibit dopaminergic neurone-specific promoter activity which is at least 15%, 20%, 25%, 30%, 40%, 50%, 60%, 70%, 80%, 90%, 100%, 125%, 150%, 175%, 200%, 250%, 300%, 350% or 400% of the activity exhibited by tyrosine hydroxylase in dopaminergic neurones. Activity of a given synthetic CNS-specific promoter of the present invention compared to tyrosine kinase can be assessed by comparing dopaminergic neurone-specific expression of a reporter gene under control of the synthetic CNS-specific promoter with expression of the same reporter under control of the tyrosine hydroxylase promoter in dopaminergic neurones, when the two promoters are provided in otherwise equivalent expression constructs and under equivalent conditions. In some embodiments a synthetic CNS-specific promoter of the invention is able to increase expression of a gene (e.g. a therapeutic gene or gene of interest) in the dopaminergic neurones of a subject by at least 20%, at least 40%, at least 60%, at least 80%, at least 100%, at least 200%, at least 300%, at least 500%, at least 1000% or more relative to a known dopaminergic neurone-specific promoter, suitably the tyrosine hydroxylase promoter.

Alternatively, it might be preferred to have a widespread expression in all or almost all regions of the brain. This may be the case, for example, in the treatment of diseases such as Angelman syndrome where a widespread expression throughout the brain is necessary.

In some embodiments a synthetic CNS-specific promoter of the invention is able to increase expression of a gene (e.g. a therapeutic gene or gene of interest) in the CNS of a subject or in a CNS cell by at least 20%, at least 40%, at least 60%, at least 80%, at least 100%, at least 200%, at least 300%, at least 500%, at least 1000% or more relative to a known CNS-specific promoter, suitably the Syn1, Camk2a or NSE promoter.

Preferred synthetic CNS-specific promoters of the present invention exhibit activity in non-CNS cells (e.g. Huh7 and HEK293 cells) which is 50% or less when compared to CMV-IE, preferably 25% or less than CMV-IE, more preferably 10% or less than CMV-IE, and in some cases 5% or less than CMV-IE, or 1% or less than CMV-IE.

In many instances, shorter promoter sequences are preferred, particularly for use in situations where a vector (e.g. a viral vector such as AAV) has limited capacity. Accordingly, in some embodiments the synthetic CNS-specific promoter has length of 1000 or fewer nucleotides, for example, 900, 800, 700,600, 500, 450, 400, 350, 300, 250, 200, 150, 100, or fewer nucleotides.

Particularly preferred synthetic CNS-specific promoters are those that are both short and which exhibit high levels of activity.

It is surprising when a CNS-specific promoter retains CNS-specific activity in an AAV vector as the AAV vector's ITRs and different DNA structure compared to the genome are known to influence the activity of promoters, often the ITRs and different DNA structure negatively impact the activity of promoters.

Synthetic CNS-Specific Expression Cassettes

The present invention also provides a synthetic CNS-specific expression cassette comprising a synthetic CNS-specific promoter of the present invention operably linked to a sequence encoding an expression product, suitably a gene (e.g. a transgene).

Where the gene encodes a protein, it can be essentially any type of protein. By way of non-limiting example, the protein can be an enzyme, an antibody or antibody fragment (e.g. a monoclonal antibody), a viral protein (e.g. REP-CAP, REV, VSV-G, or RD114), a therapeutic protein, or a toxic protein (e.g. Caspase 3, 8 or 9).

In some preferred embodiments of the present invention, the gene encodes a therapeutic expression product, preferably a therapeutic polypeptide suitable for use in treating a disease or condition associated with aberrant gene expression, optionally in the CNS.

In some embodiments, therapeutic expression products include those useful in the treatment of CNS diseases. The term “CNS disease” is, in principle, understood by the skilled person. The term relates to a disease amenable to treatment and/or prevention by administration of an active compound to the CNS, in particular to a CNS cell. In some embodiments, the CNS disease is a neurological disease and/or disorder.

As a non-limiting example, the CNS disease may be selected from: Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt- Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavemous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia—Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado—Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy—Congenital, Myopathy—Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurosyphilis, Neurotoxicity, Nevus Cavernosus, Niemann-Pick Disease, O′Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry -Romberg, Pelizaeus—Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke- Korsakoff Syndrome, West Syndrome, Whiplash, Whipple's Disease, Williams Syndrome, Wilson Disease, Wolman's Disease, X-Linked Spinal and Bulbar Muscular Atrophy.

In some embodiments, the CNS disease is selected from the list consisting of: dopamine transporter deficiency syndrome, an attention deficit/hyperactivity disorder (ADHD), bipolar disorder, epilepsy, multiple sclerosis, tauopathies, Alzheimer's disease, Huntington's disease, Parkinson's disease, Krabbe's disease, adrenoleukodystrophy, motor neurone disease, cerebral palsy, Batten disease, Gaucher disease, Tay Sachs disease, Rett syndrome, Sandhoff disease, Charcot-Marie-Tooth disease, Angelman syndrome, Canavan disease, Late infantile neuronal ceroid lipofuscinosis, Mucopolysaccharidosis IIIA, Mucopolysaccharidosis IIIB, Metachromatic leukodystrophy, heritable lysosomal storage diseases such as Niemann-Pick disease type C1, and/or neuronal ceroid lipofuscinoses such as Batten disease, progressive supranuclear palsy, corticobasal syndrome, and brain cancer (including astrocytomas and glioblastomas).

Various expression products suitable for treating the above conditions have been described in the art. Suitably, the nucleic acid encoding an expression product operably linked to the CRE, minimal/proximal promoter or promoter according to the invention may be one of the genes selected from the group consisting of: NPC1, EAAT2, NPY, CYP46A1, GLB1, APOE (e.g. ApoE2, ApoE3 or ApoE4), HEX, CLN1, CLN2, CLN3, CLN4, CLN5, CLN6, SUMF1, DCTN1, PRPH, SOD1, NEFH, GBA, IDUA, NAGLU, GUSB, ARSA, MANB, AADC, GDNF, NTN, ASP, MECP2, PTCHD1, GJB1, UBE3A, HEXA, MOG. Additionally, or alternatively, expression product operably linked to the CRE, minimal/proximal promoter or promoter according to the invention may the miRNA/CRISPR Cas9 directed to the disease allele.

CYP46A1 is the rate-limiting enzyme for cholesterol degradation and it has been found to play a beneficial role in multiple CNS diseases. CYP46A1 inhibition may contribute to inducing and/or aggravating Alzheimer's disease via increased amount of viral cholesterol, as described in (Djelti et al., 2015) which is incorporated herein by reference. CYP46A1 has also been found to be neuroprotective in Huntington's disease, as described in (Boussicault et al., 2016) which is incorporated herein by reference. Therefore, the CYP46A1 gene is a particularly preferred nucleic acid encoding an expression product. In some preferred embodiments, the CYP46A1 gene is operably linked to the CRE, minimal/proximal promoter or promoter according to the invention. Suitably, the CYP46A1 gene is operably linked to a synthetic promoter which is active in all areas of the CNS (pan-CNS) or a promoter which is active in more than 5, 6, 7, 8 or 9 of the areas of the brain recited above. Expression of CYP45A1 in all areas of the CNS or more than 5, 6, 7, 8 or 9 of the areas of the brain recited above may be beneficial as CYP46A1 expression by the ubiquitous promoters CMV or CAG was found to be beneficial in a mouse Huntington's disease model (Kacher et al., 2019). Suitably, the CYP46A1 gene is operably linked to a synthetic promoter consisting or comprising of SEQ ID NO: 1, SEQ ID NO: 21 or SEQ ID NO: 2.

In some embodiments, useful expression products include dystrophins (including micro-dystrophins), beta 1,4-n-acetylgalactosamine galactosyltransferase (GALGT2), carbamoyl synthetase I, alpha-1 antitrypsin, ornithine transcarbamylase, arginosuccinate synthetase, arginosuccinate lyase, arginase, fumarylacetacetate hydrolase, phenylalanine hydroxylase, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, and a cystic fibrosis transmembrane regulator (CFTR).

Still other useful expression products include enzymes useful in enzyme replacement therapy, and which are useful in a variety of conditions resulting from deficient activity of enzyme. For example, enzymes containing mannose-6-phosphate may be utilized in therapies for lysosomal storage diseases (e.g., a suitable gene includes that encoding β-glucuronidase (GUSB)).

In some embodiments, exemplary polypeptide expression products include neuroprotective polypeptides and anti-angiogenic polypeptides. Suitable polypeptides include, but are not limited to, glial derived neurotrophic factor (GDNF), fibroblast growth factor 2 (FGF-2), nurturin, ciliary neurotrophic factor (CNTF), nerve growth factor (NGF; e.g., nerve growth factor-. beta.), brain derived neurotrophic factor (BDNF), neurotrophin-3 (NT-3), neurotrophin-4 (NT-4), neurotrophin-6 (NT-6), epidermal growth factor (EGF), pigment epithelium derived factor (PEDF), a Wnt polypeptide, soluble Fit-1 , angiostatin, endostatin, VEGF, an anti-VEGF antibody, a soluble VEGFR, Factor VIII (FVIII), Factor IX (FIX), and a member of the hedgehog family (sonic hedgehog, Indian hedgehog, and desert hedgehog, etc.).

In some embodiments, useful therapeutic expression product include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet- derived growth factor (PDGF), insulin growth factors I and II (IGF-I and IGF-II), any one of the transforming growth factor alpha superfamily, including TGFa., activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

In some embodiments, useful expression products include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors alpha and beta., interferons (alpha, beta, and gamma), stem cell factor, flk-2/flt3 ligand. Gene products produced by the immune system are also useful in the present invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

In some embodiments, useful expression product include any one of the receptors for the hormones, growth factors, cytokines, lymphokines, regulatory proteins and immune system proteins. Useful heterologous nucleic acid sequences also include receptors for cholesterol regulation and/or lipid modulation, including the low-density lipoprotein (LDL) receptor, high density lipoprotein (HDL) receptor, the very low density lipoprotein (VLDL) receptor, and scavenger receptors. The invention also encompasses the use of gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP-2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

In some embodiments, useful expression products include non-naturally occurring polypeptides, such as chimeric or hybrid polypeptides having a non-naturally occurring amino acid sequence containing insertions, deletions or amino acid substitutions.

Further suitable expression products include micro RNA (miRNA), interfering RNA, antisense RNA, ribozymes, and aptamers.

In some embodiments of the invention, the synthetic CNS-specific expression cassette comprises a gene useful for gene editing, e.g. a gene encoding a site-specific nuclease, such as a meganuclease, zinc finger nuclease (ZFN), transcription activator-like effector-based nuclease (TALEN), or the clustered regularly interspaced short palindromic repeats system (CRISPR-Cas). Suitably the site-specific nuclease is adapted to edit a desired target genomic locus by making a cut (typically a site-specific double-strand break) which is then repaired via non-homologous end-joining (NHEJ) or homology dependent repair (HDR), resulting in a desired edit. The edit can be the partial or complete repair of a gene that is dysfunctional, or the knock-down or knock-out of a functional gene. Alternatively, the edit can be via base editing or prime editing, using suitable systems which are known in the art.

Suitably the synthetic CNS-specific expression cassette comprises sequences providing or coding for one or more of, and preferably all of, a ribosomal binding site, a start codon, a stop codon, and a transcription termination sequence. Suitably the expression cassette comprises a nucleic acid encoding a posttranscriptional regulatory element. Suitably the expression cassette comprises a nucleic acid encoding a polyA element.

Vectors and Viral Particles

The present invention further provides a vector comprising a synthetic CNS-specific promoter, or expression cassette according to the present invention.

In some embodiments of the invention, the vector is a plasmid. Such a plasmid may include a variety of other functional nucleic acid sequences, such as one or more selectable markers, one or more origins of replication, multiple cloning sites and the like. In some embodiments of the invention, the vector is a viral vector.

In some embodiments of the invention, the vector is an expression vector for expression in eukaryotic cells. Examples of eukaryotic expression vectors include, but are not limited to, pW-LNEO, pSV2CAT, pOG44, pXTI and pSG available from Stratagene; pSVK3, pBPV, pMSG and pSVL available from Amersham Pharmacia Biotech; and pCMVDsRed2-express, pIRES2-DsRed2, pDsRed2-Mito, pCMV-EGFP available from Clontech. Many other vectors are well-known and commercially available. For mammalian cells adenoviral vectors, the pSV and the pCMV series of vectors are particularly well-known non-limiting examples. There are many well-known yeast expression vectors including, without limitation, yeast integrative plasmids (Ylp) and yeast replicative plasmids (YRp). For plants the Ti plasmid of agrobacterium is an exemplary expression vector, and plant viruses also provide suitable expression vectors, e.g. tobacco mosaic virus (TMV), potato virus X, and cowpea mosaic virus.

In some preferred embodiments, the vector is a gene therapy vector. Various gene therapy vectors are known in the art, and mention can be made of AAV vectors, adenoviral vectors, retroviral vectors and lentiviral vectors. Where the vector is a gene therapy vector the vector preferably comprises a nucleic acid sequence operably linked to the synthetic CNS-specific promoter of the invention that encodes a therapeutic product, suitably a therapeutic protein. The therapeutic protein may be a secretable protein. Non-limiting examples of secretable proteins are discussed above, and exemplary secretable therapeutic proteins, include clotting factors, such as factor VIII or factor IX, insulin, erythropoietin, lipoprotein lipase, antibodies or nanobodies, growth factors, cytokines, chemokines, plasma factors, toxic proteins, etc.

In some embodiments of the invention, the vector is a viral vector, such as a retroviral, lentiviral, adenoviral, herpes simplex or adeno-associated viral (AAV) vector. In some preferred embodiments, the vector is a lentiviral vector, suitably a lentiviral vector based on HIV-1. In some preferred embodiments the vector is an AAV vector. In some preferred embodiments the AAV has a serotype suitable or specifically optimised for CNS transduction. In some embodiments, the AAV is selected from the group consisting of: AAV1, AAV2, AAV4, AAV5, AAV8, AAV9, AAVrh10, AAVDJ8 and AAV2g9, or derivatives thereof.

AAV vectors are preferably used as self-complementary, double-stranded AAV vectors (scAAV) in order to overcome one of the limiting steps in AAV transduction (i.e. single-stranded to double-stranded AAV conversion), although the use of single-stranded AAV vectors (ssAAV) is also encompassed herein. In some embodiments of the invention, the AAV vector is chimeric, meaning it comprises components from at least two AAV serotypes, such as the ITRs of an AAV2 and the capsid protein of an AAV5. AAV9 is known to effectively transduce CNS cells and tissue particularly effectively, and thus AAV9 and derivatives thereof are of particular interest for targeting CNS cells and tissue. AAV2g9 is known to effectively transduce CNS cells and tissue particularly effectively, and thus AAV2g9 and derivatives thereof are of particular interest for targeting CNS cells and tissue. AAVrh10 is known to effectively transduce CNS cells and tissue particularly effectively, and thus AAVrh10 and derivatives thereof are of particular interest for targeting CNS cells and tissue. AAVrh10 is particularly preferred as systemic or intravenous delivery of AAVrh10 has been found to provide high transgene expression in the central nervous system as described in (Tanguy et al., 2015) which is incorporated herein by reference. AAVDJ8 is known to effectively transduce CNS cells and tissue particularly effectively, and thus AAVDJ8 and derivatives thereof are of particular interest for targeting CNS cells and tissue. AAVDJ8 is preferred as it has been shown to effectively target multiple regions of the brain and to effectively target astrocytes as described in (Hammond et al., 2017) which is incorporated herein by reference. AAV1, AAV2, AAV4, AAV5 and AAV8 are also known to target CNS cells and tissue, and thus these AAV serotypes and derivates thereof are also of particular interest for targeting CNS cells and tissue.

The invention further provides recombinant virions (viral particles) comprising a vector as described above.

Pharmaceutical Compositions

The vectors or virions of the present invention may be formulated in a pharmaceutical composition with a pharmaceutically acceptable excipient, i.e., one or more pharmaceutically acceptable carrier substances and/or additives, e.g., buffers, carriers, excipients, stabilisers, etc. The pharmaceutical composition may be provided in the form of a kit. Pharmaceutical compositions and delivery systems appropriate for the AAV vectors or and methods and uses of are known in the art.

Accordingly, a further aspect of the invention provides a pharmaceutical composition comprising a vector or virion as described herein.

Relative amounts of the active ingredient (e.g. AAV vector particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1 percent and 99 percent (w/w) of the active ingredient. By way of example, the composition may comprise between 0.1 percent and 100 percent, e.g., between.5 and 50 percent, between 1-30 percent, between 5-80 percent, at least 80 percent (w/w) active ingredient.

The pharmaceutical compositions can be formulated using one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the invention. In some embodiments, a pharmaceutically acceptable excipient may be at least 95 percent, at least 96 percent, at least 97 percent, at least 98 percent, at least 99 percent, or 100 percent pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia. Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21 st Edition, A. R. Gennaro, Lippincott, Williams and Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Therapeutic and Other Methods and Uses

The present invention also provides a synthetic CNS-specific promoter, expression cassette, vector, virion or pharmaceutical composition according to various aspects of the present invention for use in the treatment of a disease, preferably a disease associated with aberrant gene expression, optionally in the CNS (e.g. a genetic CNS disease). Relevant conditions, diseases and therapeutic expression products are discussed above.

The present invention also provides a synthetic CNS-specific promoter, expression cassette, vector, virion according to the various aspects of the present invention for use as medicament.

The present invention also provides a synthetic CNS-specific promoter, expression cassette, vector, virion according to the various aspects of the present invention for use the manufacture of a pharmaceutical composition for treatment of any condition or disease mentioned herein.

The present invention further provides a cell comprising a synthetic CNS-specific promoter, expression cassette, vector, virion according to the various aspects of the invention. Suitably the cell is a eukaryotic cell. The eukaryotic cell can suitably be an animal (metazoan) cell (e.g. a mammalian cell). Suitably, the cell is a human cell.

In some embodiments of the invention, the cell is ex vivo, e.g. in cell culture. In other embodiments of the invention the cell may be part of a tissue or multicellular organism.

In a preferred embodiment, the cell is a CNS cell, which may be ex vivo or in vivo. The CNS cell may be a primary neurone, astrocyte, oligodendrocyte, microglial cell or an ependymal cell. Alternatively, the CNS cell may be a CNS-derived cell line, e.g. immortalised cell line.

The cell may be present within a CNS tissue environment (e.g. within the CNS of an animal) or may be isolated from CNS tissue, e.g. it may be in cell culture. Suitably the primary cell or the cell line is a human cell.

The synthetic CNS-specific promoter, expression cassette, or vector, according to the invention may be inserted into the genome of the cell, or it may be episomal (e.g. present in an episomal vector).

In a further aspect the present invention provides a method for producing an expression product, the method comprising providing a synthetic CNS-specific expression cassette according to the present invention (preferably in a vector as set out above) in a cell, preferably a CNS cell, and expressing the gene present in the synthetic CNS-specific expression cassette. The method suitably comprises maintaining said CNS cell under suitable conditions for expression of the gene. In culture this may comprise incubating the cell, or tissue comprising the cell, under suitable culture conditions. The expression may of course be in vivo, e.g. in one or more cells in the CNS of a subject.

Suitably the method comprises the step of introducing the synthetic CNS-specific expression cassette into the CNS cell. A wide range of methods of transfecting CNS cells are well-known in the art. A preferred method of transfecting CNS cells is transducing the cells with a viral vector comprising the synthetic CNS-specific expression cassette, e.g. an AAV vector.

It will be evident to the skilled person that a synthetic CNS-specific promoter, expression cassette, vector or virion according to various aspects of the invention may be used for gene therapy. Accordingly, the use of the such nucleic acid constructs in gene therapy forms part of the present invention.

The invention thus provides, in some embodiments, an expression cassette, vector or virion according to the present invention for use in gene therapy in a subject, preferably gene therapy through CNS-specific expression of a therapeutic gene. The therapy may involve treatment of a disease through secretion of a therapeutic product from CNS cells, suitably a disease involving aberrant gene expression in the CNS, as discussed above.

The present invention also provides a method of expressing a therapeutic transgene in a CNS cell, the method comprising introducing into the CNS cell an expression cassette or vector according to the present invention. The CNS cell can be in vivo or ex vivo.

The present invention also provides a method of gene therapy of a subject, preferably a human, in need thereof, the method comprising:
administering to the subject (suitably introducing into the CNS of the subject) a synthetic CNS-specific expression cassette, vector, virion or pharmaceutical composition of the present invention, which comprises a gene encoding a therapeutic product.

The method suitably comprises expressing a therapeutic amount of the therapeutic product from the gene in the CNS of said subject. Various conditions and diseases that can be treated are discussed above. Genes encoding suitable therapeutic products are discussed above.

The method suitably comprises administering a vector or virion according to the present invention to the subject. Suitably the vector is a viral gene therapy vector, for example an AAV vector.

In some embodiments, the method comprises administering the gene therapy vector systemically. Systemic administration may be enteral (e.g. oral, sublingual, and rectal) or parenteral (e.g. injection). Preferred routes of injection include intravenous, intramuscular, subcutaneous, intra-arterial, intra-articular, intrathecal, and intradermal injections. In one embodiment, the gene therapy vector may be delivered by injection into the CSF pathway. Non-limiting examples of delivery to the CSF pathway include intrathecal and intracerebroventricular administration.

Particularly preferred route of administration of AAV vector or virion comprising the synthetic CNS-specific promoter or expression cassette according to this invention is intravascular. Suitably, the AAV vector or virion comprising the synthetic CNS-specific promoter or expression cassette according to this invention may be administered in the veins of the dorsal hand or the veins of the anterior forearm. Suitable veins in the anterior forearm are the cephalic, median or basilic veins. This is because this administration route is generally safe for the patient while still allowing some penetration into the CNS.

In some embodiments, the viral gene therapy vector may be administered concurrently or sequentially with one or more additional therapeutic agents or with one or more saturating agents designed to prevent clearance of the vectors by the reticular endothelial system.

Where the vector is an AAV vector, the dosage of the vector may be from 1×10¹⁰ gc/kg to 1×10¹⁵ gc/kg or more, suitably from 1×10¹² gc/kg to 1×10¹⁴ gc/kg, suitably from 5×10¹² gc/kg to 5×10¹³ gc/kg.

In general, the subject in need thereof will be a mammal, and preferably a primate, more preferably a human. Typically, the subject in need thereof will display symptoms characteristic of a disease. The method typically comprises ameliorating the symptoms displayed by the subject in need thereof, by expressing the therapeutic amount of the therapeutic product. In one embodiment, the therapeutic methods of the present invention may be used to reduce the decline of functional capacity and activities of daily living as measured by a standard evaluation system such as, but not limited to, the total functional capacity (TFC) scale. In one embodiment, the methods of the present invention may be used to improve performance on any assessment used to measure symptoms of neurological disease. Such assessments include, but are not limited to ADAS-cog (Alzheimer Disease Assessment Scale—cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE—AD, EuroQol, Short Form-36 and/or MBR Caregiver Strain Instrument, or any of the other tests as described in Sheehan B (Ther Adv Neurol Disord. 5(6):349-358 (2012)), the contents of which are herein incorporated by reference in their entirety.

Gene therapy protocols for therapeutic gene expression in target cells in vitro and in vivo, are well-known in the art and will not be discussed in detail here. Briefly, they include intravenous or intraarterial administration (e.g. intra-corotid artery, intra-hepatic artery, intra-hepatic vein), intracranial administration, intramuscular injection, interstitial injection, instillation in airways, application to endothelium and intra-hepatic parenchyme, of plasmid DNA vectors (naked or in liposomes) or viral vectors. Various devices have been developed for enhancing the availability of DNA to the target cell. While a simple approach is to contact the target cell physically with catheters or implantable materials containing the relevant vector, more complex approaches can use jet injection devices an suchlike. Gene transfer into mammalian CNS cells can been performed using both ex vivo and in vivo procedures. The ex vivo approach typically requires harvesting of the CNS cells, in vitro transduction with suitable expression vectors, followed by reintroduction of the transduced CNS cells into the CNS. This approach is generally less preferred due to the difficulty and danger of harvesting and reintroducing CNS cells in the brain. In vivo gene transfer has been achieved by injecting DNA or viral vectors directly into the CNS, e.g. by intracranial injection, or by intravenous or intraarterial injection of viral vectors.

In one embodiment, the gene therapy vector may be administered to a subject (e.g., to the CNS of a subject) in a therapeutically effective amount to reduce the symptoms of neurological disease of a subject (e.g., determined using a known evaluation method). In some embodiments, the gene therapy vector and compositions comprising the gene therapy vector may be administered in a way which allows them to cross the blood-brain barrier, vascular barrier, or other epithelial barrier.

The gene therapy vectors may be used in combination with one or more other therapeutic, prophylactic, research or diagnostic agents. By “in combination with,” it is not intended to imply that the agents must be administered at the same time and/or formulated for delivery together, although these methods of delivery are within the scope of the present invention. Compositions can be administered concurrently with, prior to, or subsequent to, one or more other desired therapeutics or medical procedures. Compounds which may be used in combination with the AAV particles described herein include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK3 (lithium) or PP2A, immunization with?beta peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acety choline release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or Zolpidem for altered sleep-wake cycle), anticonvulsants (e.g., sodium valproate and carbamazepine for mania or hypomania) and mood stabilizers (e.g., lithium for mania or hypomania).

According to some preferred embodiments, the methods set out above may be used for the treatment of a subject with a CNS-related disease as discussed above, e.g. dopamine transporter deficiency syndrome.

Definitions and General Points

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds. -in-chief, Academic Press, Inc., New York), specifically, Vols.154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

The term “central nervous system” or “CNS” is well understood by the skilled person. The CNS consists of the brain and the spinal cord. Preferably, the synthetic CNS-specific promoters are active in the brain. The promoters of the present invention can be active in brain and/or spinal cord. Preferably, the CNS is a CNS of a mammal, even more preferably of a human subject.

The term “CNS cell” or “CNS cells” relates to cells which are found in CNS (CNS tissue) or which are derived from CNS tissue. CNS cells can be primary cells or a cell line (such as SH-Sy5y, Neuro2A, U87-MG). The CNS cells can be in in vivo (e.g. in CNS tissue) or in vitro (e.g. in cell culture). CNS cells comprise of neurones, astrocytes, oligodendrocytes, microglial cells and ependymal cells. Neurones as found in the CNS tissue comprise a cell body, a long axon and a synaptic terminal. A neurone transmits electric signals received in the cell body via its long axon to other cells close to their synaptic terminal. Oligodendrocytes are a type of glial cell in the CNS which produces myelin sheaths which rap around neuronal axon for faster electrical signal conduction. Astrocytes are star-shaped and are the most abundant cell type in the brain. They have multiple roles which aid and regulate transmission of electrical impulses within the brain and neuronal function. Microglia are the resident macrophage cell in the brain and are involved in immune defence. Ependymal cells form the epithelial lining of the ventricles. The term “CNS cell” or “CNS cells” as used herein includes neurones, astrocytes, oligodendrocytes, microglial cells and/or ependymal cells. The promoters of the present invention can be active in any of the CNS cell (e.g. neurones). The promoters of the present invention may be active in more than one type of CNS cell (e.g. neurones and astrocytes). The promoters of the present invention may be active in all types of CNS cells (neurones, astrocytes, oligodendrocytes, microglial cells and ependymal cells). Additionally, synthetic CNS-specific promoters of the present invention may be active in a subtype of a type of CNS cell such as dopaminergic neurones or mature oligodendrocytes. In some embodiments, the synthetic CNS-specific promoters of the present invention may only be active in the subtype of a type of CNS cell such as dopaminergic neurones or mature oligodendrocytes. The CREs, proximal/minimal promoters and promoters of the present invention may be active in specific areas of the CNS, in specific CNS cells or CNS cell subtypes or both. In some embodiments, the CREs, proximal/minimal promoters and promoters of the present invention may be active in a specific CNS cell type, such as neurones, within all areas of the CNS. In other embodiments, the CREs, proximal/minimal promoters and promoters of the present invention may be active in a specific CNS cell type, such as neurones, within no more than one area of the CNS, such as midbrain. In some embodiments, the CREs, proximal/minimal promoters and promoters of the present invention may be active in all CNS cells in all areas of the CNS. In some embodiments, the CREs, proximal/minimal promoters and promoters of the present invention may be active in al CNS cells in no more than one area of the CNS, such as midbrain.

The term “cis-regulatory element” or “CRE”, is a term well-known to the skilled person, and means a nucleic acid sequence such as an enhancer, promoter, insulator, or silencer, that can regulate or modulate the transcription of a neighbouring gene (i.e. in cis). CREs are found in the vicinity of the genes that they regulate. CREs typically regulate gene transcription by binding to TFs, i.e. they include TFBS. A single TF may bind to many CREs, and hence control the expression of many genes (pleiotropy). CREs are usually, but not always, located upstream of the transcription start site (TSS) of the gene that they regulate. “Enhancers” in the present context are CREs that enhance (i.e. upregulate) the transcription of genes that they are operably associated with, and can be found upstream, downstream, and even within the introns of the gene that they regulate. Multiple enhancers can act in a coordinated fashion to regulate transcription of one gene. “Silencers” in this context relates to CREs that bind TFs called repressors, which act to prevent or downregulate transcription of a gene. The term “silencer” can also refer to a region in the 3′ untranslated region of messenger RNA, that bind proteins which suppress translation of that mRNA molecule, but this usage is distinct from its use in describing a CRE. Generally, the CREs of the present invention are CNS-specific enhancer elements (often referred to as CNS-specific CREs, or CNS-specific CRE enhancers, or suchlike). In the present context, it is preferred that the CRE is located 2500 nucleotides or less from the transcription start site (TSS), more preferably 2000 nucleotides or less from the TSS, more preferably 1500 nucleotides or less from the TSS, and suitably 1000, 750, 500, 250, 200, 150, or 100 nucleotides or less from the TSS. CREs of the present invention are preferably comparatively short in length, preferably 1000 nucleotides or less in length, for example they may be 800, 700, 600, 500, 400, 300, 200, 175, 150, 90, 80, 70, 60 or 50 nucleotides or less in length. The CREs of the present invention are typically provided in combination with an operably linked promoter element, which ca be a minimal promoter or proximal promoter; the CREs of the present invention may enhance CNS-specific activity of the promoter element.

The term “cis-regulatory module” or “CRM” means a functional regulatory nucleic acid module, which usually comprises two or more CREs; in the present invention the CREs are typically CNS-specific enhancers and thus the CRM is a synthetic CNS-specific regulatory nucleic acid. A CRM may comprise a plurality of CNS-specific CREs. Suitably, at least one of the CREs comprised in the CRM is a CRE according to SEQ ID NO: 9-11, 28-31 or a functional variant thereof. Typically, the multiple CREs within the CRM act together (e.g. additively or synergistically) to enhance the transcription of a gene that a promoter comprising the CRM is operably associated with. There is considerable scope to shuffle (i.e. reorder), invert (i.e. reverse orientation), and alter spacing of CREs within a CRM. Accordingly, functional variants of CRMs of the present invention include, inter alia, variants of the referenced CRMs wherein CREs within them have been shuffled and/or inverted, and/or the spacing between CREs has been altered.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. Many diverse promoters are known in the art.

The term “synthetic promoter” as used herein relates to a promoter that does not occur in nature. In the present context it typically comprises a CRE and/or CRM of the present invention operably linked to a minimal (or core) promoter or CNS-specific proximal promoter (promoter element). The CREs and/or CRMs of the present invention serve to enhance CNS-specific transcription of a gene operably linked to the synthetic promoter. Parts of the synthetic promoter may be naturally occurring (e.g. the minimal promoter or one or more CREs in the promoter), but the synthetic promoter as an entity is not naturally occurring. Alternatively, the synthetic promoter may be a shorter, truncated version of a promoter which occurs in nature.

As used herein, “minimal promoter” (also known as the “core promoter”) refers to a typically short DNA segment which is inactive or largely inactive by itself, but can mediate transcription when combined with other transcription regulatory elements. Minimal promoter sequences can be derived from various different sources, including prokaryotic and eukaryotic genes. Examples of minimal promoters include SYNP_CRE151 (SEQ ID NO: 12). Other examples of minimal promoters are the dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box). A minimal promoter may also include some elements downstream of the TSS, but these typically have little functionality absent additional regulatory elements.

As used herein, “proximal promoter” relates to the minimal promoter plus at least some additional regulatory sequence, typically the proximal sequence upstream of the gene that tends to contain primary regulatory elements. It often extends approximately 250 base pairs upstream of the TSS, and includes specific TFBS. A proximal promoter may also include one or more regulatory elements downstream of the TSS, for example a UTR or an intron. In the present case, the proximal promoter may suitably be a shorter, truncated version of naturally occurring CNS-specific proximal promoter. The proximal promoters of the present invention may be combined with one or more CREs or CRMs of the present invention. However, the proximal promoter can also be synthetic.

As used herein, “promoter element” refers to either a minimal promoter or proximal promoter as defined above. In the context of the present invention a promoter element may be combined with one or more CREs in order to provide a synthetic CNS-specific promoter of the present invention.

A “functional variant” of a CRE, CRM, promoter element, promoter or other regulatory nucleic acid in the context of the present invention is a variant of a reference sequence that retains the ability to function in the same way as the reference sequence, e.g. as a CNS-specific CRE, CNS-specific CRM or CNS-specific promoter. Alternative terms for such functional variants include “biological equivalents” or “equivalents”.

It will be appreciated that the ability of a given CRE, CRM, promoter or other regulatory sequence to function as a CNS-specific enhancer is determined significantly by the ability of the sequence to bind the same CNS-specific TFs that bind to the reference sequence. Accordingly, in most cases, a functional variant of a CRE or CRM will contain TFBS for the most or all of same TFs as the reference CRE, CRM or promoter. It is preferred, but not essential, that the TFBS of a functional variant are in the same relative positions (i.e. order and general position) as the reference CRE, CRM or promoter. It is also preferred, but not essential, that the TFBS of a functional variant are in the same orientation as the reference sequence (it will be noted that TFBS can in some cases be present in reverse orientation, e.g. as the reverse complement vis-a-vis the sequence in the reference sequence). It is also preferred, but not essential, that the TFBS of a functional variant are on the same strand as the reference sequence. Thus, in preferred embodiments, the functional variant comprises TFBS for the same TFs, in the same order, the same position, in the same orientation and on the same strand as the reference sequence. It will also be appreciated that the sequences lying between TFBS (referred to in some cases as spacer sequences, or suchlike) are of less consequence to the function of the CRE or CRM. Such sequences can typically be varied considerably, and their lengths can be altered. However, in preferred embodiments the spacing (i.e. the distance between adjacent TFBS) is substantially the same (e.g. it does not vary by more than 20%, preferably by not more than 10%, and more preferably it is approximately the same) in a functional variant as it is in the reference sequence. It will be apparent that in some cases a functional variant of a CRE can be present in the reverse orientation, e.g. it can be the reverse complement of a CRE as described above, or a variant thereof.

Levels of sequence identity between a functional variant and the reference sequence can also be an indicator or retained functionality. High levels of sequence identity in the TFBS of the CRE, CRM or promoter is of generally higher importance than sequence identity in the spacer sequences (where there is little or no requirement for any conservation of sequence). However, it will be appreciated that even within the TFBS, a considerable degree of sequence variation can be accommodated, given that the sequence of a functional TFBS does not need to exactly match the consensus sequence.

The ability of one or more TFs to bind to a TFBS in a given functional variant can determined by any relevant means known in the art, including, but not limited to, electromobility shift assays (EMSA), binding assays, chromatin immunoprecipitation (ChIP), and ChIP-sequencing (ChIP-seq). In a preferred embodiment the ability of one or more TFs to bind a given functional variant is determined by EMSA. Methods of performing EMSA are well-known in the art. Suitable approaches are described in Sambrook et al. cited above. Many relevant articles describing this procedure are available, e.g. Hellman and Fried, Nat Protoc. 2007; 2(8): 1849-1861.

“CNS-specific” or “CNS-specific expression” refers to the ability of a cis-regulatory element, cis-regulatory module or promoter to enhance or drive expression of a gene in CNS cells (or in CNS-derived cells) in a preferential or predominant manner as compared to other tissues (e.g. liver, kidney, spleen, heart, muscle and lung). Expression of the gene can be in the form of mRNA or protein. In preferred embodiments, CNS-specific expression is such that there is negligible expression in other (i.e. non-CNS) tissues or cells, i.e. expression is highly CNS-specific.

The ability of a CRE, CRM or promoter to function as a CNS-specific CRE, CRM or promoter can be readily assessed by the skilled person. The skilled person can thus easily determine whether any variant of the specific CRE, CRM or promoter recited above remains functional (i.e. it is a functional variant as defined above). For example, any given CRM to be assessed can be operably linked to a minimal promoter (e.g. positioned upstream of CMV-MP or upstream of SEQ ID NO: 12 or 13) and the ability of the cis-regulatory element to drive CNS-specific expression of a gene (typically a reporter gene) is measured. Alternatively, a variant of a CRE or CRM can be substituted into a synthetic CNS-specific promoter in place of a reference CRE or CRM, and the effects on CNS-specific expression driven by said modified promoter can be determined and compared to the unmodified form. Similarly, the ability of a promoter to drive CNS-specific expression can be readily assessed by the skilled person (e.g. as described in the examples below). Expression levels of a gene driven by a variant of a reference promoter can be compared to the expression levels driven by the reference promoter. In some embodiments, where CNS-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess CNS-specific expression enhancement can be easily constructed, and the examples set out below gives suitable methodologies.

CNS-specificity can be identified wherein the expression of a gene (e.g. a therapeutic or reporter gene) occurs preferentially or predominantly in CNS-derived cells. Preferential or predominant expression can be defined, for example, where the level of expression is significantly greater in CNS-derived cells than in other types of cells (i.e. non-CNS-derived cells). For example, expression in CNS-derived cells is suitably at least 5-fold higher than in non-CNS cells, preferably at least 10-fold higher than in non-CNS cells, and it may be 50-fold higher or more in some cases. For convenience, CNS-specific expression can suitably be demonstrated via a comparison of expression levels in a different non-CNS cell lines, e.g. primary CNS cells or CNS-derived cell line such as SH-Sy5y, Neuro2A, U87-MG compared with expression level in a muscle-derived cell line such as C2C12 or H2K cells (skeletal muscle) or H9C2 cells (cardiac), in a liver-derived cell line (e.g. Huh7 or HepG2), kidney-derived cell line (e.g. HEK-293), a cervical tissue-derived cell line (e.g. HeLa) and/or a lung-derived cell line (e.g. A549).

The synthetic CNS-specific promoters of the present invention preferably exhibit reduced expression in non-CNS-derived cells, suitably in C2C12, H9C2, Huh7, HEK-293, HeLa, and/or A549 cells when compared to a non-tissue specific promoter such as CMV-I E. The synthetic CNS-specific promoters of the present invention preferably have an activity of 50% or less than the CMV-IE promoter in non-CNS-derived cells (suitably in C2C12, H9C2, Huh7, HEK-293, HeLa, and/or A549), suitably 25% or less, 20% or less, 15% or less, 10% or less, 5% or less or 1% or less. Generally, it is preferred that expression in non-CNS-derived cells is minimized, but in some cases this may not be necessary. Even if a synthetic CNS-specific promoter of the present invention has higher expression in, e.g., one or two non-CNS cells, as long as it generally has higher expression overall in a range of CNS cells versus non-CNS cell, it can still be a CNS-specific promoter.

The synthetic CNS-specific promoters of the present invention are preferably suitable for promoting expression in the CNS of a subject, e.g. driving CNS-specific expression of a transgene, preferably a therapeutic transgene. Preferred synthetic CNS-specific promoters of the present invention are suitable for promoting CNS-specific transgene expression and have an activity in CNS cells which is at least 15%, 20%, 30%, 40%, 50%, 60%, 70%, 80%, 90%, 100%, 125%, 150%, 175%, 200%, 250%, 300%, 350% or 400% of the activity of the Synapsin-1 promoter. In some embodiments, the synthetic CNS-specific promoters of the invention are suitable for promoting CNS-specific transgene expression at a level at least 100% of the activity of the Synapsin-1 promoter, preferably 150%, 200%, 300% or 500% of the activity of the Synapsin-1 promoter. Such CNS-specific expression is suitably determined in CNS-derived cells, e.g. SH-Sy5y, Neuro2A, U87-MG cell lines or primary CNS cells (suitably primary human neurones, astrocytes, oligodendrocytes, microglia and/or ependymal cells).

Synthetic CNS-specific promoters of the present invention may also be able to promote CNS-specific expression of a gene at a level at least 50%, 100%, 150% or 200% compared to CMV-IE in CNS-derived cells, e.g. SH-Sy5y, Neuro2A, U87-MG cell lines or primary CNS cells (suitably primary human neurones, astrocytes, oligodendrocytes, microglia and/or ependymal cells).

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

By “isolated” is meant, when referring to a nucleic acid is a nucleic acid molecule or a nucleic acid sequence devoid, in whole or part, of sequences normally associated with it in nature; or a sequence, as it exists in nature, but having heterologous sequences in association therewith; or a molecule disassociated from the chromosome.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “transcription factor binding site” (TFBS) is well known in the art. It will be apparent to the skilled person that TFBS sequences can be modified, provided that they are bound by the intended transcription factor (TF). Consensus sequences for the various TFBS disclosed herein are known in the art, and the skilled person can readily use this information to determine alternative TFBS. Furthermore, the ability of a TF to bind to a given putative sequence can readily be determined experimentally by the skilled person (e.g. by EMSA and other approaches well known in the art and discussed herein).

The meaning of “consensus sequence” is well-known in the art. In the present application, the following notation is used for the consensus sequences, unless the context dictates otherwise. Considering the following exemplary DNA sequence:

A[CT]N{A}YR

A means that an A is always found in that position; [CT] stands for either C or T in that position; N stands for any base in that position; and {A} means any base except A is found in that position. Y represents any pyrimidine, and R indicates any purine.

“Synthetic” in the present application means a nucleic acid molecule that does not occur in nature. Synthetic nucleic acids of the present invention are produced artificially, typically by recombinant technologies or de novo synthesis. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

“Complementary” or “complementarity”, as used herein, refers to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to its complementary base.

The term “administration” as used herein refers to introduction of a foreign substance into the human or animal body. Administration can be, for example, intravenous, intraarterial or intracranial.

“Transfection” in the present application refers broadly to any process of deliberately introducing nucleic acids into cells, and covers introduction of viral and non-viral vectors, and includes or is equivalent to transformation, transduction and like terms and processes. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; whiskers-mediated transformation; and microprojectile bombardment (Klein et al. (1987) Nature 327:70).

As used herein, the phrase “transgene” refers to an exogenous nucleic acid sequence. In one example, a transgene is a gene encoding an industrially or pharmaceutically useful compound, or a gene encoding a desirable trait. In yet another example, the transgene encodes useful nucleic acid such as an antisense nucleic acid sequence, wherein expression of the antisense nucleic acid sequence inhibits expression of a target nucleic acid sequence. The transgene preferably encodes a therapeutic product, e.g. a protein.

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated. Vectors of the present invention can be episomal vectors (i.e., that do not integrate into the genome of a host cell), or can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts. Viral vectors are derived from viruses and include but are not limited to retroviral, lentiviral, adeno-associated viral, adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco)lysis. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

The term “operably linked”, “operably connected” or equivalent expressions as used herein refer to the arrangement of various nucleic acid elements relative to each other such that the elements are functionally connected and are able to interact with each other in the manner intended. Such elements may include, without limitation, a promoter, a CRE (e.g. enhancer or other regulatory element), a promoter element, a polyadenylation sequence, one or more introns and/or exons, and a coding sequence of a gene of interest to be expressed. The nucleic acid sequence elements, when properly oriented or operably linked, act together to modulate the activity of one another, and ultimately may affect the level of expression of an expression product. By modulate is meant increasing, decreasing, or maintaining the level of activity of a particular element. The position of each element relative to other elements may be expressed in terms of the 5′ terminus and the 3′ terminus of each element or their position upstream or downstream of another element or position (such as a TSS or promoter element), and the distance between any particular elements may be referenced by the number of intervening nucleotides, or base pairs, between the elements. As understood by the skilled person, operably linked implies functional activity, and is not necessarily related to a natural positional link. Indeed, when used in nucleic acid expression cassettes, CREs will typically be located immediately upstream of the promoter element (although this is generally the case, it should definitely not be interpreted as a limitation or exclusion of positions within the nucleic acid expression cassette), but this needs not be the case in vivo, e.g., a regulatory element sequence naturally occurring downstream of a gene whose transcription it affects is able to function in the same way when located upstream of the promoter. Hence, according to a specific embodiment, the regulatory or enhancing effect of the regulatory element can be position- independent.

A “spacer sequence” or “spacer” as used herein is a nucleic acid sequence that separates two functional nucleic acid sequences (e.g. TFBS, CREs, CRMs, promoter element, etc.). It can have essentially any sequence, provided it does not prevent the functional nucleic acid sequence (e.g. cis-regulatory element) from functioning as desired (e.g. this could happen if it includes a silencer sequence, prevents binding of the desired transcription factor, or suchlike). Typically, it is non-functional, as in it is present only to space adjacent functional nucleic acid sequences from one another. In some embodiments, spacers may have a length of 75, 50, 40, 30, 30 or 10 nucleotides or fewer.

The term “pharmaceutically acceptable” as used herein is consistent with the art and means compatible with the other ingredients of the pharmaceutical composition and not deleterious to the recipient thereof.

“Therapeutically effective amount” and like phrases mean a dose or plasma concentration in a subject that provides the desired specific pharmacological effect, e.g. to express a therapeutic gene in the CNS. A therapeutically effective amount may not always be effective in treating the conditions described herein, even though such dosage is deemed to be a therapeutically effective amount by those of skill in the art. The therapeutically effective amount may vary based on the route of administration and dosage form, the age and weight of the subject, and/or the disease or condition being treated.

The term “AAV vector” as used herein is well known in the art, and generally refers to an AAV vector nucleic acid sequence including various nucleic acid sequences. An AAV vector as used herein typically comprise a heterologous nucleic acid sequence not of AAV origin as part of the vector. This heterologous nucleic acid sequence typically comprises a promoter as disclosed herein as well as other sequences of interest for the genetic transformation of a cell. In general, the heterologous nucleic acid sequence is flanked by at least one, and generally by two AAV inverted terminal repeat sequences (ITRs). An “AAV virion” or “AAV virus” or “AAV viral particle” or “AAV vector particle” refers to a viral particle composed of at least one AAV capsid polypeptide (including both variant AAV capsid polypeptides and non-variant parent capsid polypeptides) and an encapsidated polynucleotide AAV vector. If the particle comprises a heterologous nucleic acid (i.e. a polynucleotide other than a wild-type AAV genome, such as a transgene to be delivered to a mammalian cell), it can be referred to as an “AAV vector particle” or simply an “AAV vector”. Thus, production of AAV virion or AAV particle necessarily includes production of AAV vector as such a vector is contained within an AAV virion or AAV particle. The ITRs may be derived from the same serotype as the capsid, selected from any of the serotypes listed in Table 1, or may be from a different serotype than the capsid. The AAV vector typically has more than one ITR. In a non-limiting example, the AAV vector has a viral genome comprising two ITRs. In one embodiment, the ITRs are of the same serotype as one another. In another embodiment, the ITRs are of different serotypes. Non-limiting examples include zero, one or both of the ITRs having the same serotype as the capsid. Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In one embodiment, the ITRs are 140-142 nucleotides in length. Non-limiting examples of ITR length are 102, 105, 130, 140, 141, 142, 145 nucleotides in length.

As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome capable of modulating the productive utilization of mRNA. An artificial microRNA can be any type of RNA sequence, other than endogenous microRNA, capable of modulating the activity of an mRNA. A microRNA sequence can be an RNA molecule composed of any one or more of these sequences. MicroRNA (or “miRNA”) sequences have been described in publications such as Lim, et al , 2003, Genes & Development, 17, 991-1008, Lim et al , 2003, Science, 299, 1540, Lee and Ambrose, 2001, Science, 294, 862, Lau et al, 2001, Science 294, 858-861, Lagos-Quintana et al, 2002, Current Biology, 12, 735-739, Lagos- Quintana ei a/., 2001, Science, 294, 853-857, and Lagos-Quintana et al. , 2003, RNA, 9, 175-179. Examples of microRNAs include any RNA fragment of a larger RNA or is a miRNA, siRNA, stRNA, sncRNA, tncRNA, snoRNA, smRNA, shRNA, snRNA, or other small non-coding RNA. See, e.g., U.S. Patent Applications 20050272923, 20050266552, 20050142581, and 20050075492. A “microRNA precursor” (or “pre-miRNA”) refers to a nucleic acid having a stem-loop structure with a microRNA sequence incorporated therein. A “mature microRNA” (or “mature miRNA”) includes a microRNA cleaved from a microRNA precursor (a “pre-miRNA”), or synthesized (e.g., synthesized in a laboratory by cell-free synthesis), and has a length of from about 19 nucleotides to about 27 nucleotides, e.g. , a mature microRNA can have a length of 19 nt, 20 nt, 21 nt, 22 nt, 23 nt, 24 nt, 25 nt, 26 nt, or 27 nt. A mature microRNA can bind to a target mRNA and inhibit translation of the target mRNA.

The terms “treatment” or “treating” refer to reducing, ameliorating or eliminating one or more signs, symptoms, or effects of a disease or condition. “Treatment,” as used herein thus includes any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease.

The “administration” of an agent to a subject includes any route of introducing or delivering to a subject the agent to perform its intended function. Administration can be carried out by any suitable route, including orally, intranasally, intraocularly, ophthalmically, parenterally (intravascularly, intramuscularly, intraperitoneally, or subcutaneously), or topically. Administration includes self-administration and the administration by another. Intravenous or intraarterial administration is of particular interest in the present invention.

The terms “individual,” “subject,” and “patient” are used interchangeably, and refer to any individual subject with a disease or condition in need of treatment. For the purposes of the present disclosure, the subject may be a primate, preferably a human, or another mammal, such as a dog, cat, horse, pig, goat, or bovine, and the like.

The term “specifically active in an area or in a tissue” refers to a promoter which is predominantly active in that area or tissue, i.e. more active in that area or tissue than in other areas or tissues.

### EXAMPLES

### Example 1

CNS transduction and vector biodistribution of CNS1-8 (SEQ ID NO: 1-4, 23-26) operably linked to GFP were studied using AAV9.

AAV plasmid preparation:

hSyn.GFP plasmid containing ssAAV2 inverted terminal repeats was obtained from Addgene and used to generate the control AAV vector (Synapsin-1). The CNS 1-8 (SEQ ID NO: 1-4, 23-26) promoters were cloned into hSyn.GFP plasmid to replace hSyn promoter by GeneArt® (Thermo Fisher Scientific, Germany). All plasmid DNA was prepared using PureLink™ HiPure Plasmid Maxiprep Kit (#K210007; Thermo Fisher Scientific, Germany) according to manufacturer instructions and quantified on an Omega FLUOstar spectrophotometer (BMG Labtech, UK).

AAV vector preparation:

Recombinant AAV2/9 (referred to as AAV9, throughout) vectors encoding GFP were generated by the standard triple plasmid transfection method. Briefly, viral producer human embryonic kidney (HEK) 293T cells were co-transfected with three plasmids, pGFP controlled by different promoters (SEQ ID NO: 1-4, 23-26), pGD9 encoding AAV9 capsid and pHGTI containing the helper functions using polyethylenimine (PEI) (#24765; Polysciences, UK), at stock concentration 1 mg/ml, in molar proportion 1:3:1. After 72 hours, the cells were collected and lysed. Cell lysate and supernatant were nuclease treated, filtered and purified through affinity chromatography on an ÄKTAprime plus (GE Healthcare Ltd, UK) with Primeview 5.0 software with a POROS™ CaptureSelect™ AAVX resin (Thermo Fisher Scientific, Germany).

AAV Vector Titration: All vector preparations were titred by qPCR to the GFP transgene following Luna® Universal qPCR Master Mix manufacturer's instructions (#M3003; New England Biolabs, UK) on a QuantStudio™ 3 System Real-Time PCR (Thermo Fisher Scientific, UK). Data were analysed using QuantStudio design and analysis software V5. Primers designed to amplify a segment of the GFP transgene (Table 5) were used to determine the number of vector genomes. All vectors were titre-matched to 1×10¹³ vector genomes/mL (vg/mL).

Animal Procedures:

All animal experiments were performed in compliance with UK Home Office regulations and the Animals (Scientific Procedures) Act 1986 within the guidelines of University College London ethical review committee. Outbred CD1 mice (Charles River, UK) were housed at the Central Biological Services Unit, UCL in individually ventilated cages (IVC) cages, under standard conditions, with a 12 hours light-dark cycle, constant temperature (21-23° C.), humidity (60%±5), access to pelleted food and water ad libitum. Experimental breeding pairs were time-mated after 6 weeks of age and newborn litters used for these promoter studies. Pups were weaned at P21 and euthanised for tissue analysis at P35.

Animal Injections:

All pups were injected on the day of birth (P0). The pups were subject to transient hypothermic anaesthesia prior to either method of injection. For each injection method, four mice were injected per vector type with 4 uninjected controls, each identified uniquely by paw tattooing. The pups were warmed to normal temperature before returning to the dam.

Neonatal Intracranial Injections of Viral Vectors:

Pups were injected with 5 μl viral vector (5×10¹⁰ viral genomes/pup) into the cerebral lateral ventricles using 33 gauge Hamilton needle (Fisher Scientific, UK) using established coordinates (Kim, Ji-Yoen et al, 2013) which is incorporated herein by reference. Injection into the ventricles bypasses the blood brain barrier.

Neonatal Intravenous Injections of Viral Vectors:

Pups were injected with 20 μl viral vector (2×10¹¹ vg/pup) into the superficial temporal vein. The vein was visualised using fiber optic transillumination and injections performed using a 33 gauge Hamilton needle, using a stereoscopic dissecting microscope (Zeiss, Germany).

Perfusion and Tissue Preparation:

The animals were anaesthetised with isoflurane (5% induction chamber, 1.5% maintenance via nose cone). Transcardial perfusion was performed by cutting the right atrium and injecting the left ventricle with 10 mL autoclaved PBS (Phosphate-buffered saline) until hepatic blanching was achieved. Brains and visceral organs were halved to allow for different processing techniques depending on the following experiments. The halves used for immunohistochemistry were post-fixed in 4% Paraformaldehyde (PFA) for 48 hours and transferred into 30% sucrose solution for cryoprotection at 4° C. until sectioning. Half brains were mounted on a freezing microtome (Thermo Fisher HM430) at 40 mm thickness in either coronal or sagittal planes and stored in TBSAF (Tris-buffered saline (TBS), 30% ethylene glycol, 15% sucrose, 0.05% sodium azide) at 4° C. Brain halves and visceral organ tissues used for molecular biology evaluation experiments, were snap frozen in dry ice and stored at −80° C. Standard DNA and/or RNA extraction protocols were followed to perform vector copy number (VCN) and gene expression (cDNA) qPCR analyses, respectively.

Tissue Aanalysis of GFP Expression:

GFP expression in the mouse brains was assessed through immunohistochemistry (IHC) and immunofluorescence (IHF).

Free Floating IHC with Diaminobenzidine (DAB) Immunoperoxidase Stain:

Brain sections were selected for either whole brain analysis or representative sections from different brain regions (olfactory bulb, prefrontal cortex, striatum, hippocampus, midbrain and cerebellum). All wash steps were performed three times in 1×TBS at room temperature (RT).

All brain sections were washed prior to treatment with 30% H₂O₂ (Sigma Aldrich, UK) in 1×TBS for 30 minutes and blocked with 15% normal goat serum (Vector Laboratories, UK) in TBST (1×TBS, 0.3% Triton X-100) for 30 minutes at RT. Samples were incubated for 12-14 hours in the primary antibodies (rabbit or chicken anti-GFP antibodies from Table 6) at 4° C. with constant agitation on an orbital shaker. The sections were washed and incubated for 2 hours at RT with the corresponding biotinylated secondary antibody (anti-rabbit or anti-chicken biotinylated secondary antibodies from Table 6) on an orbital shaker. Sections were washed and incubated with Vectastain avidin-biotin solution (ABC Vector Stain, Vector Laboratories, UK). The sections were washed and the reaction visualised with DAB (Sigma Aldrich, UK) (10 mg DAB in 20 mL TBS, 6m1 30% H202). The reaction was stopped after a maximum of 7 minutes using ice cold 1×TBS before mounting on glass slides.

Free-Floating Immunofluorescence:

A similar protocol as the DAB immunoperoxidase stain was used. The sections were washed in 1×TBS and blocked in 15% normal goat serum for 30 minutes. The sections were incubated with primary antibodies of choice (transgene marker and cell type marker, rabbit/chicken anti-GFP and rabbit/chicken anti-tyrosine hydroxylase from Table 6) diluted in 10% normal goat serum TBST and incubated overnight at 4° C. The sections were washed in TBS and incubated for 2 hours in secondary fluorophores diluted in 10% normal goat serum covered at RT (anti-chicken/rabbit Alexa flour secondary antibodies from Table 6). The sections were washed and treated with DAPI (4′,6-diamidino-2-phenylindole, Sigma Aldrich, UK) for 2 minutes and transferred to ice cold 1×TBS and then mounted on glass slides.

Microscopy:

Light microscopy and fluorescence imaging was carried out using a Leica DM4000B and all images captured using a Leica DFC420 camera and Leica Application Suite V3.7 software maintaining light intensity, exposure, microscope calibration and photo camera settings constant (Leica Microsystems, UK).

Quantitative measurement of 10 non-overlapping RGB images at ×40 magnification of GFP staining intensity was performed by thresholding analysis on selected brain regions: cortex, hippocampus, striatum, midbrain and cerebellum. The foreground immunostaining was defined by averaging of the highest and lowest signals and the mean percentage area of immunoreactivity per field for each region of interest was calculated using Image-Pro 10 software (Media Cybernetics, USA).

Quantification of midbrain dopaminergic (mDA) neurons was conducted by counting of TH-positive neurons and vector-driven GFP-expressing cells and a percentage of double-positive neurons was calculated.

qRT-PCR for Vector Expression Analysis:

RNA was extracted from brains and organs using the TRIzol™ Plus RNA Purification Kit (Thermo Fisher Scientific, Germany) or the RNeasy mini kit (Qiagen, UK), and quantified on Omega FLUOstar (BMG Labtech, UK). Contaminating DNA was removed from total RNA (1-2 μg) using the DNAse I purification kit (NEB, UK), before performing reverse transcription with High-Capacity cDNA Reverse Transcription Kit (Applied Bioscience, Thermo Fisher Scientific, Germany). 10 ng cDNA was used to perform the qPCR with Luna Taqman mastermix (NEB, UK) with 300 nM primers (Table 5) on a QuantstudioTM Real-Time PCR System (Applied Biosystems, UK).

For the quantification of GFP transcripts, standardisation was achieved by comparison against standard curves generated by amplification from plasmid constructs specific for GFP and mGAPDH transcripts. mGAPDH was used as endogenous control and relative fold change calculated as described for vector genome copy number analysis.

TABLE 5
Primers sequences used for viral vector
titration and qRT-PCR

| | |
|---|---|
| GFP F | GGCACAAGCTGGAGTACAAC |
| | (SEQ ID NO: 15) |
| GFP R | AGTTCACCTTGATGCCGTTC |
| | (SEQ ID NO: 16) |
| GFP Probe | (FAM)-AGCCACAACGTCTATATCATGGCCG |
| | (SEQ ID NO: 17) |
| mouseGAPDH F | ACGGCAAATTCAACGGCAC |
| | (SEQ ID NO: 18) |
| mouseGAPDH R | TAGTGGGGTCTCGCTCCTGG |
| | (SEQ ID NO: 19) |
| mouseGAPDH Probe | (VIC)-TTGTCATCAACGGGAAGCCCATCA |
| | (SEQ ID NO: 20) |

TABLE 6
Antibodies used for IHC or IHF

| Antibody | Dilution | Manufacturer reference |
|---|---|---|
| Rabbit anti-Green Fluorescent Protein | 1:10000 | Abeam, UK; #AB290 |
| Chicken anti-Green Fluorescent Protein | 1:1000 | Aves Labs, UK; #GFP-1010 |
| Mouse anti-NeuN | 1:500 | Merck Millipore, |
| | | UK; #MAB377 |
| Mouse anti-Glial Fibrillary Acidic Protein | 1:500 | Merck Millipore, UK; |
| Antibody | | #MAB3402 |
| Rabbit anti-IBA1 | 1:2000 | Wako, Japan; #019-19741 |
| Chicken anti-Tyrosine hydroxylase | 1:500 | Aves Labs, UK; #TYH |
| Rabbit anti-Tyrosine hydroxylase | 1:500 | Merck Millipore, UK; #AB152 |
| Biotinylated secondary antibodies | 1:1000 | Vector Laboratories, UK |
| Secondary Alexa fluor | 1:500 | Thermo Fisher Scientific, UK |

CNS 1-8 Construct Design

The promoters in the present invention were designed by a mixture of bioinformatic analysis and literature review.

CNS-5_v2, CNS-6_v2, CNS-7_v2 and CNS-8_v2 (SEQ ID NO: 5-8) are longer versions of promoters CNS-5, CNS-6, CNS-7 and CNS-8 (SEQ ID NO: 23-26). That is to say that CNS-5_v2, CNS-6_v2, CNS-7_v2 and CNS-8_v2 (SEQ ID NO: 5-8) have been shortened and a minimal promoter SYNP_CRE151 (SEQ ID NO: 12) has been added to this shorter version to arrive at CNS-5, CNS-6, CNS-7 and CNS-8 (SEQ ID NO: 23-26). Due to the high sequence similarity between the longer and shorter versions of these synthetic promoters (e.g. CNS-5_v2 and CNS-5), they may be expected to have similar expression.

Results

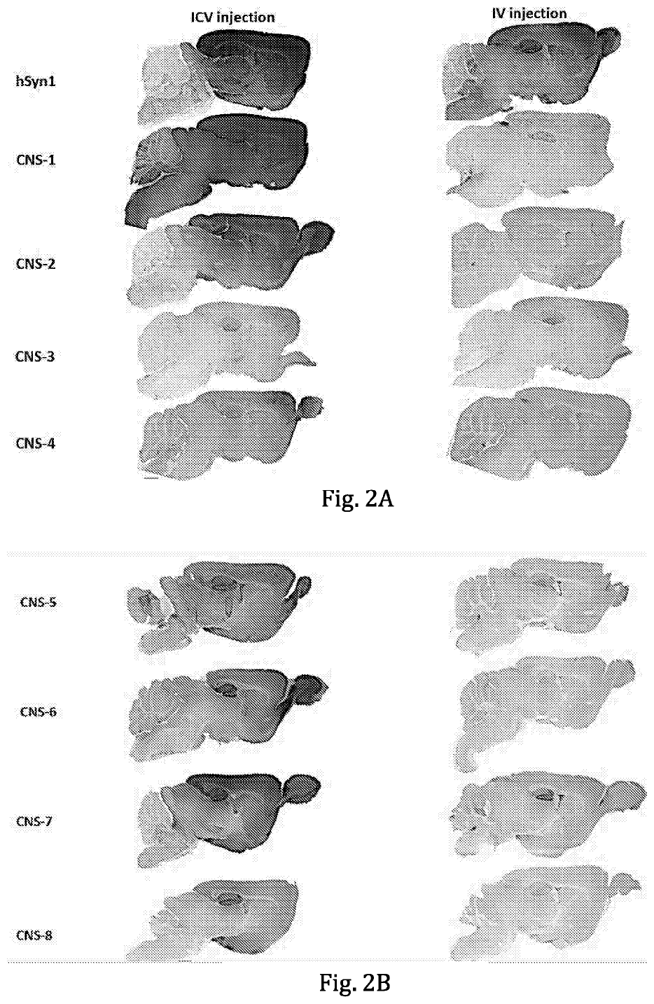

The GFP expression from CNS-1-CNS-8 promoters (SEQ ID NOs:1-4; 23-26) and from the control promoter Syn1 (SEQ ID NO:14) was initially assessed in sagittal sections and the results are shown in FIG. 2A-B. The tested promoters all show CNS expression with a range of strengths and distributions throughout different brain regions.

In ICV injected animals, CNS-1 (SEQ ID NO: 1) showed the strongest expression and CNS-3 (SEQ ID NO: 3) showed the weakest with the rest of the promoters in between the two extremes. Notably, CNS-1′s (SEQ ID NO: 1) expression was stronger and more uniform in the brain than the expression from the control promoter Syn1 (SEQ ID NO: 14).

In IV injected animals, CNS-4 (SEQ ID NO: 4) showed the strongest expression and CNS-3 (SEQ ID NO: 3) showed the weakest with the rest of the promoters in between the two extremes. Promoters CNS-1-CNS-8 (SEQ ID NOs: 1-4, 23-26) all showed weaker expression than the control promoter Syn1.

Therefore, the method of administration (ICV vs IV) impacts both the strength and the distribution of the CNS promoters.

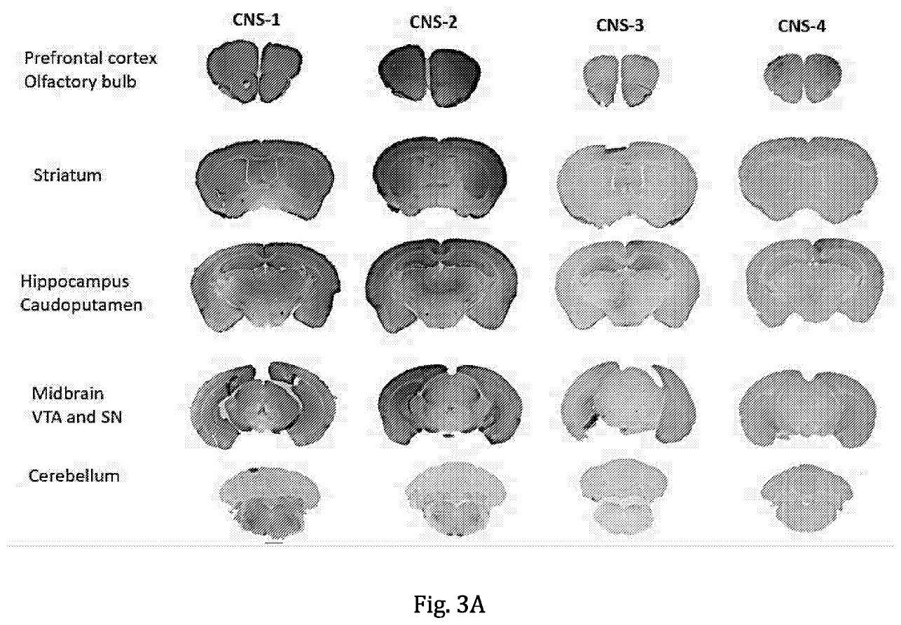

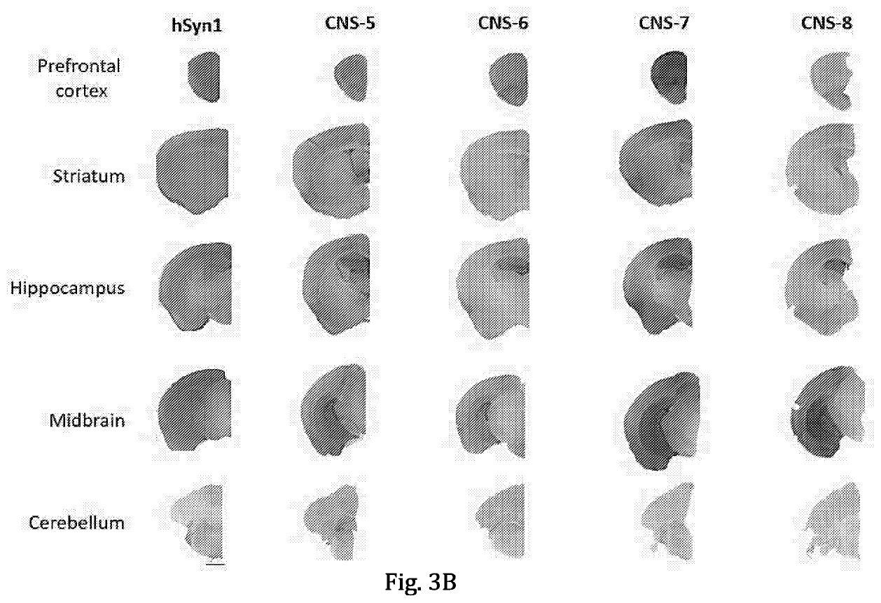

The GFP expression from CNS-1-CNS-8 (SEQ ID NO: 1-4; 23-26) promoters delivered by ICV and from the control promoter Syn1 (SEQ ID NO: 14) was then assessed in coronal sections and the results are shown in FIG. 3A-B. Again, all the tested promoters show CNS expression with a range of strengths and distributions throughout different brain regions. Promoters CNS-1, CNS-2 (SEQ ID NOs: 1-2) and CNS-7 (SEQ ID NO: 25) show the strongest expression and CNS-3 (SEQ ID NO: 3) shows the weakest expression with the rest of the promoters in between the two extremes. Promoters CNS-1 (SEQ ID NO: 1), CNS-2 (SEQ ID NO: 2) and CNS-7 (SEQ ID NO: 25) show similar expression level to control promoter Syn1.

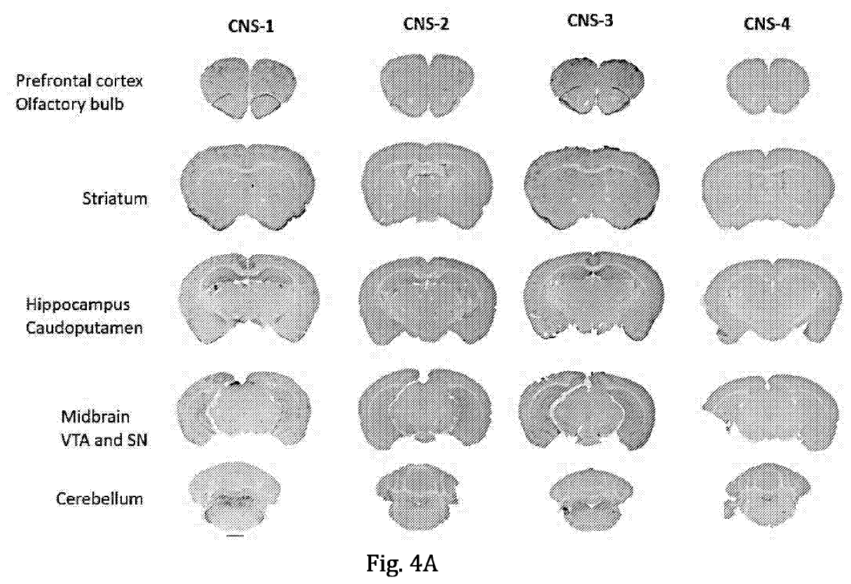

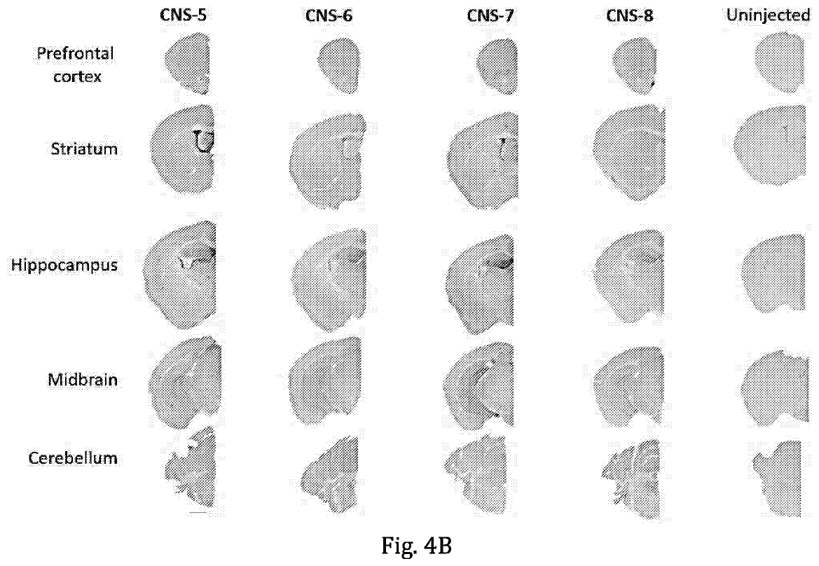

The GFP expression from the CNS-1-CNS-8 (SEQ ID NOs 1-4,23-26) promoters delivered by IV was also assessed in coronal sections and the results are shown in FIG. 4A-B. Again, all the tested promoters show CNS expression with a range of strengths and distributions throughout different brain regions. Promoter CNS-3 (SEQ ID NO: 3) shows the strongest expression and CNS-8 (SEQ ID NO: 26) shows the weakest expression with the rest of the promoters in between the two extremes.

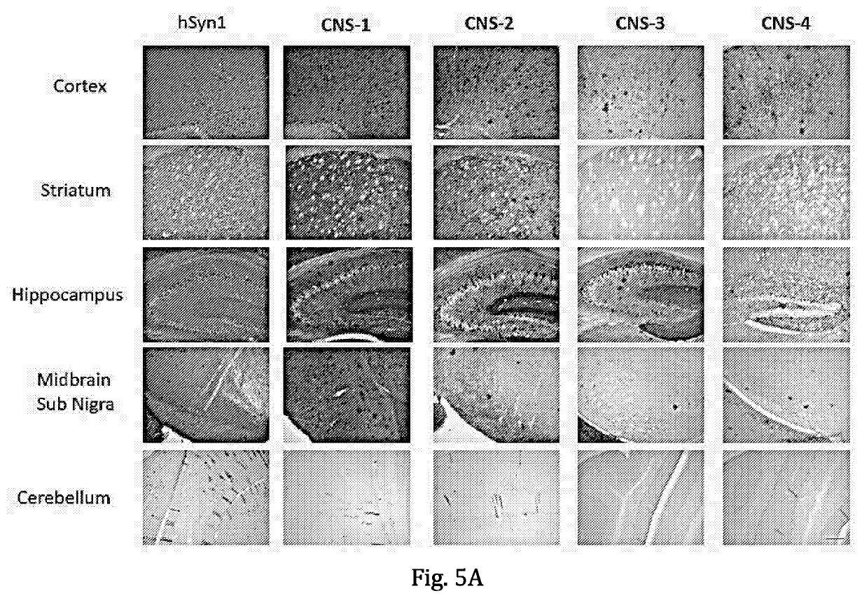

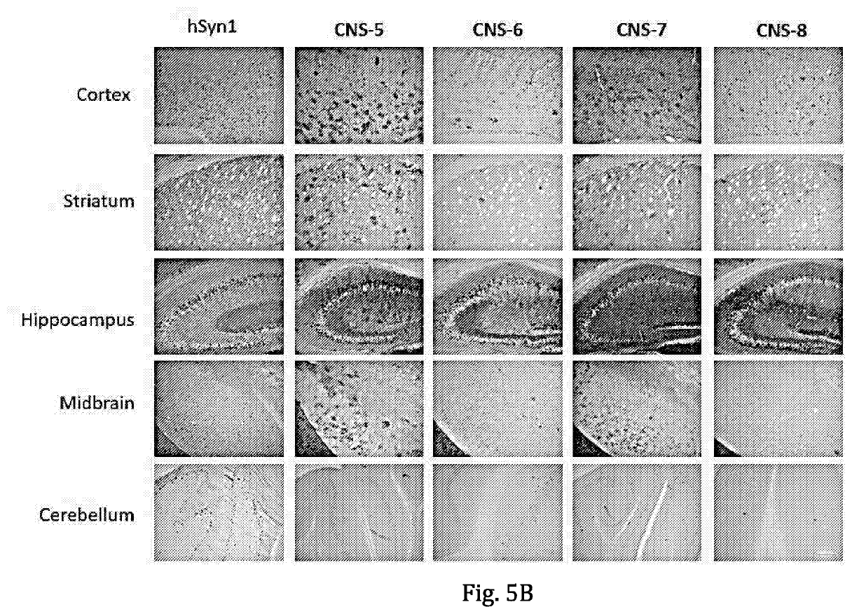

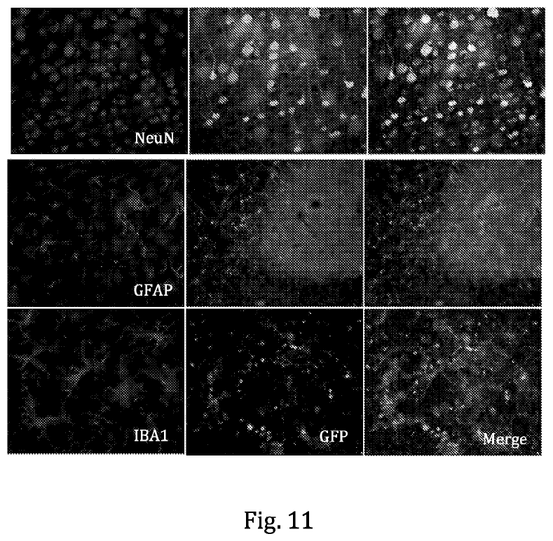

The GFP expression from CNS-1-CNS-8 (SEQ ID NO: 1-4, 23-26) promoters delivered by ICV and from the control promoter Syn1 (SEQ ID NO: 14) was then visualised at higher magnification in coronal sections and the results are shown in FIG. 5A-B. At this higher magnification, CNS-1 (SEQ ID NO: 1), CNS-2 (SEQ ID NO: 2) and CNS-7 (SEQ ID NO: 25) showed widespread intracranial expression with CNS-1 (SEQ ID NO: 1) showing the strongest expression. This expression appeared to be primarily neuronal for CNS-1 (SEQ ID NO: 1) and CNS-2 (SEQ ID NO: 2). The primarily neuronal expression of GFP when GFP is driven by CNS-1 (SEQ ID NO: 1) in ICV delivery has also been confirmed in double staining for CNS cell types as shown in FIG. 11. The expression of GFP was neuronal and astrocytic when driven by CNS-7 (SEQ ID NO: 25). CNS-3 (SEQ ID NO: 3) and CNS-4 (SEQ ID NO: 4) showed weaker expression which was localised to the cortex and the hippocampus. The expression appeared to be primarily neuronal and astrocytic for both CNS-3 (SEQ ID NO: 3) and CNS-4 (SEQ ID NO: 4). CNS-5 (SEQ ID NO: 23) was strongly active in the cortex, striatum, hippocampus and midbrain but less so in the cerebellum. CNS-6 (SEQ ID NO: 24) and CNS-8 (SEQ ID NO: 26) were strongly active in the hippocampus, followed by the cortex and the midbrain, with less expression in the other tested parts of the brain. The expression of CNS-6 (SEQ ID NO:24) and CNS-8 (SEQ ID NO: 26) appeared to be primarily neuronal.

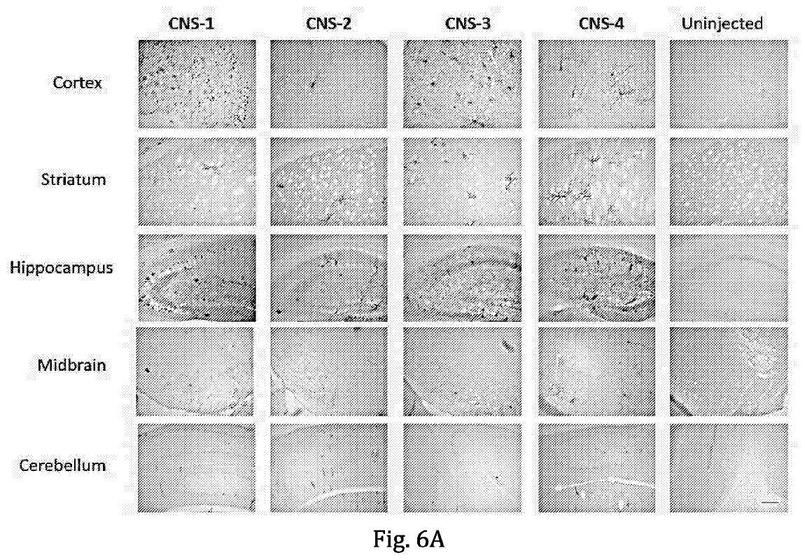

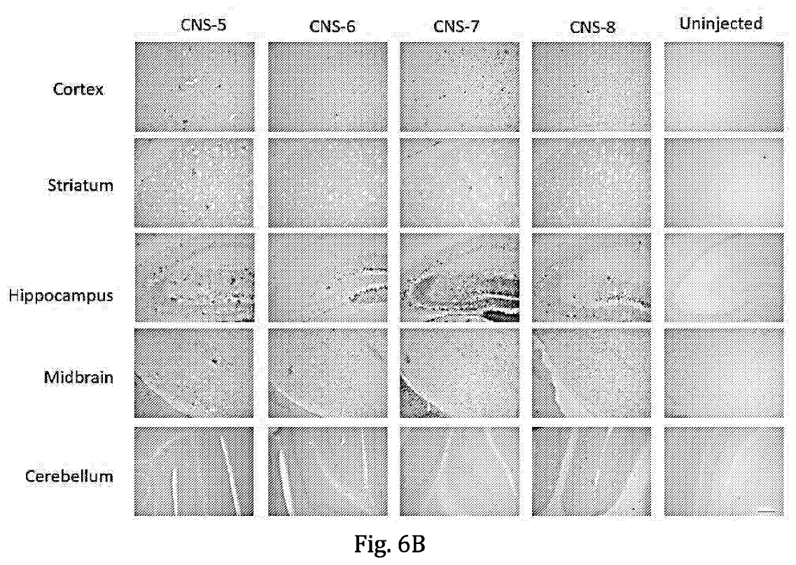

The GFP expression from CNS-1-CNS-8 (SEQ ID NO: 1-4, 23-26) promoters delivered by IV was also visualised at higher magnification in coronal sections and the results are shown in FIG. 6A-B. CNS-1 (SEQ ID NO: 1) is highly active in the cortex and the hippocampus. Minor CNS-2 (SEQ ID NO: 2) expression was seen in most tested regions apart from the midbrain. CNS-3 (SEQ ID NO: 3) and CNS-4 (SEQ ID NO: 4) showed expression in the cortex, striatum and hippocampus with CNS-4 (SEQ ID NO: 4) but not CNS-3 (SEQ ID NO: 3) showing expression in the midbrain. CNS-5 (SEQ ID NO: 23) has minimal expression in all tested areas of the brain. CNS-6 (SEQ ID NO: 24) has the expression in the hippocampus, midbrain and cerebellum. CNS-7 (SEQ ID NO: 25) shows expression in the cortex, hippocampus and midbrain. CNS-8 (SEQ ID NO: 26) is active in the hippocampus and the midbrain.

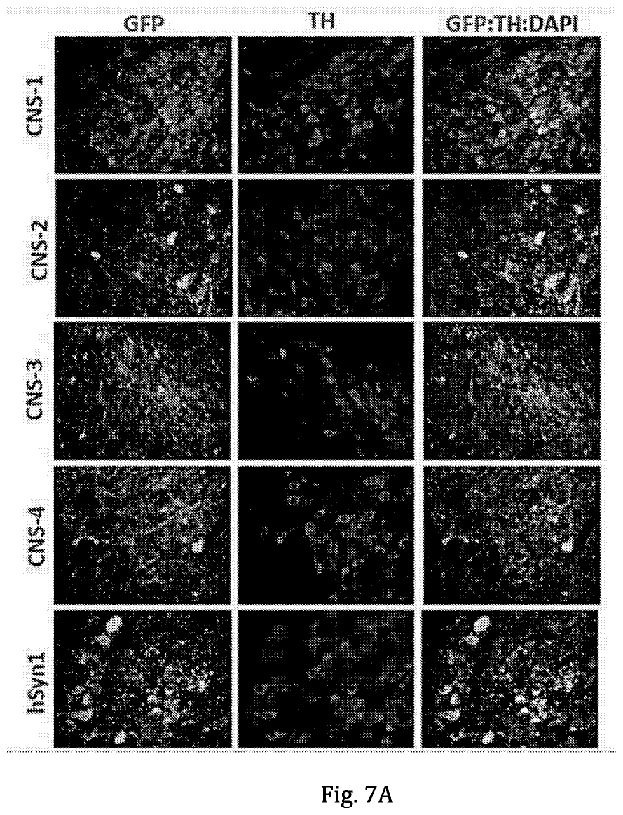

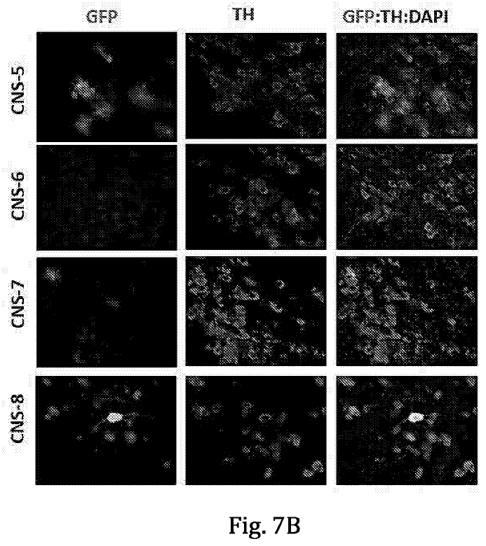

The expression from CNS1-8 (SEQ ID NO: 1-4,23-26) promoters and the control promoter Syn1 delivered by ICV was visualised at even higher magnification in the midbrain and the results are shown in FIG. 7A-B. CNS1-4 (SEQ ID NO: 1-4) and Syn1 (SEQ ID NO: 14) showed some GFP expression in the midbrain. Double staining with a marker for dopaminergic neurones (TH+) indicates that some GFP expression from the Syn1 (SEQ ID NO: 14) promoter is localised to dopaminergic neurones but only a fraction of the GFP expression from CNS1-4 (SEQ ID NO: 1-4) is localised to dopaminergic neurones. CNS-6 (SEQ ID NO: 24) and CNS-7 (SEQ ID NO: 25) showed minimal expression in the midbrain. CNS-5 (SEQ ID NO: 23) showed expression in the midbrain but that expression did not appear to be localised to dopaminergic neurones. A large proportion of the GFP expression driven by the CNS-8 (SEQ ID NO: 26) promoter is localised to dopaminergic neurones.

The expression from CNS1-8 (SEQ ID NOs: 1-4, 23-26) promoters delivered by IV was also visualised in the midbrain and the results are shown in FIG. 7A-B. CNS-1-4 (SEQ ID NO: 1-4) showed minimal GFP expression in the midbrain and the majority of the cells which were GFP positive were not dopaminergic neurones. CNS-5 (SEQ ID NO: 23), CNS-6 (SEQ ID NO: 24) and CNS-7 (SEQ ID NO: 25) did not show any GFP expression in the midbrain following IV delivery. CNS-8 (SEQ ID NO: 26), on the other hand, showed strong expression in the midbrain with many the cells showing GFP expression being dopaminergic neurones.

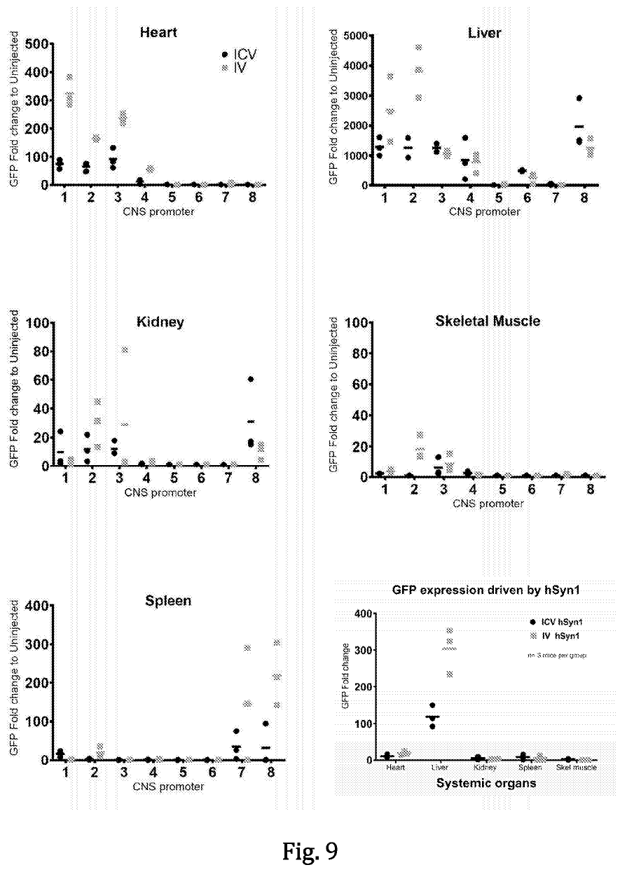

The biodistribution in different tissues of the transgene GFP under the control of CNS-1-8 (SEQ ID NOs: 1-4,23-26) and the control promoter Syn-1 (SEQ ID NO: 14) delivered by ICV and by IV are shown in FIG. 9.

In ICV delivery, CNS-1-4 (SEQ ID NOs: 1-4) show activity in the heart while the rest of the tested promoters, CNS-5-8 (SEQ ID NOs: 23-26) are not active in the heart. In IV delivery, CNS-1-4 (SEQ ID NOs: 1-4) show activity in the heart while the rest of the tested promoters, CNS-5-8 (SEQ ID NOs: 23-26) are not active in the heart. The control promoter Syn-1 also shows a very low activity in the heart in ICV delivery and IV delivery.

In ICV delivery, CNS-1-4 (SEQ ID NOs:1-4), CNS-6 (SEQ ID NO:24), CNS-8 (SEQ ID NO:26) and the control promoter Syn-1 (SEQ ID NO: 14) show activity in the liver while the rest of the tested promoters do not. In IV delivery, CNS-1-4 (SEQ ID NOs: 1-4), CNS-6 (SEQ ID NO: 24), CNS-8 (SEQ ID NO: 26) and the control promoter Syn-1 (SEQ ID NO: 14) show activity in the liver while the rest of the tested promoters do not.

In ICV delivery, CNS1-3 (SEQ ID NOs: 1-3) and CNS-8 (SEQ ID NO:26) show activity in the kidney while CNS-4-7 (SEQ ID NOs: 4, 23-25) and the control promoter Syn-1 (SEQ ID NO: 14) do not show activity in the kidney. In IV delivery, CNS-2-3 (SEQ ID NO: 2-3) and CNS-8 (SEQ ID NO: 26) show activity in the kidney while the rest of the tested promoters and the control promoter Syn-1 (SEQ ID NO: 14) do not.

In ICV delivery, CNS-3 (SEQ ID NO: 3) show activity in skeletal muscle while CNS-1-2 (SEQ ID NOs: 1-2) and CNS-4-8 (SEQ ID NO: 4, 23-26) do not show activity in skeletal muscle. In

IV delivery, CNS 1-3 (SEQ ID NOs: 1-3) show activity in skeletal muscle while CNS-4-8 (SEQ ID NOs: 4, 23-26) do not show activity in skeletal muscle. The control promoter Syn-1 (SEQ ID NO: 14) does not show activity in skeletal muscle.

In ICV delivery, CNS-1 (SEQ ID NO: 1), CNS-7-8 (SEQ ID NOs: 25-26) show activity in the spleen while CNS-2-6 (SEQ ID NO: 2-4,23-24) and the control promoter Syn-1 (SEQ ID NO: 14) do not show activity in the spleen. In IV delivery, CNS-2 (SEQ ID NO: 2) and CNS-7-8 (SEQ ID NOs: 25-26) show activity in the spleen while CNS-1 (SEQ ID NO: 1), CNS-3-6 (SEQ ID NOs: 3-4, 23-24) and the control promoter Syn-1 (SEQ ID NO: 14) do not show activity in the spleen.

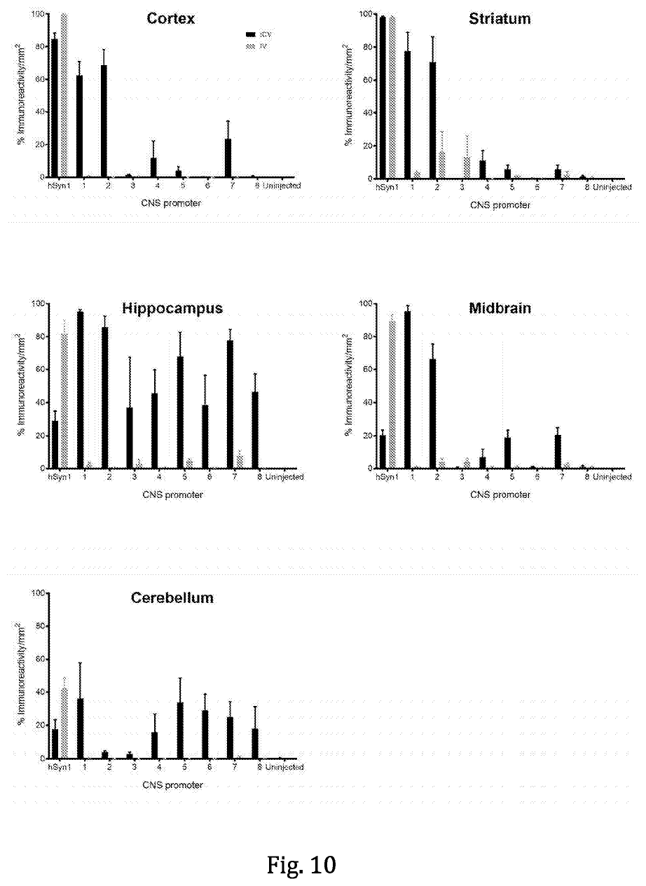

The percentage GFP immunoreactivity per mm² was measured in different area in the brain and the results are shown in FIG. 10. As before, GFP was placed under the control of CNS-1 (SEQ ID NO: 1), CNS-2 (SEQ ID NO: 2), CNS-3 (SEQ ID NO: 3), CNS-4 (SEQ ID NO: 4), CNS-5 (SEQ ID NO: 23), CNS-6 (SEQ ID NO: 24), CNS-7 (SEQ ID NO: 25), CNS-8 (SEQ ID NO: 26) and the control promoter Syn-1 (SEQ ID NO: 14) delivered by ICV and by IV. In ICV delivery, the control promoter Syn-1 (SEQ ID NO: 14), CNS-1 (SEQ ID NO: 1) and CNS-2 (SEQ ID NO: 2) had very high percentage GFP immunoreactivity in the cortex, followed by CNS-7 (SEQ ID NO: 25) and CNS-4 (SEQ ID NO: 4) with the rest of the tested promoters showing very little or no GFP immunoreactivity in the cortex. In IV delivery, the control promoter Syn-1 (SEQ ID NO: 14) had high percentage GFP immunoreactivity in the cortex but the rest of the tested promoters had very little or no GFP immunoreactivity in the cortex.

In ICV delivery, CNS-1 (SEQ ID NO: 1) and CNS-2 (SEQ ID NO: 2) had very high percentage GFP immunoreactivity in the striatum, followed by CNS-4 (SEQ ID NO: 4), CNS-5 (SEQ ID NO: 23) and CNS-7 (SEQ ID NO: 25) with the rest of the tested promoters showing very little or no GFP immunoreactivity in the striatum. In IV delivery, CNS-2 (SEQ ID NO:2) and CNS-3 (SEQ ID NO: 3) had low percentage GFP immunoreactivity in the striatum while the rest of the tested promoters had very little or no GFP immunoreactivity in the striatum. The control promoter Syn-1 (SEQ ID NO: 14) showed very high GFP immunoreactivity in the striatum in both ICV and IV delivery.

In ICV delivery, CNS-1-8 (SEQ ID NO: 1-4,23-26) showed mid or high percentage GFP immunoreactivity in the hippocampus and had higher percentage GFP immunoreactivity in the hippocampus than the control promoter Syn-1 (SEQ ID NO: 14). In IV delivery, CNS-1-8 (SEQ ID NO: 1-4,23-26) had very little or no GFP immunoreactivity in the hippocampus while the control promoter Syn-1 (SEQ ID NO: 14) showed very high GFP immunoreactivity in the hippocampus.

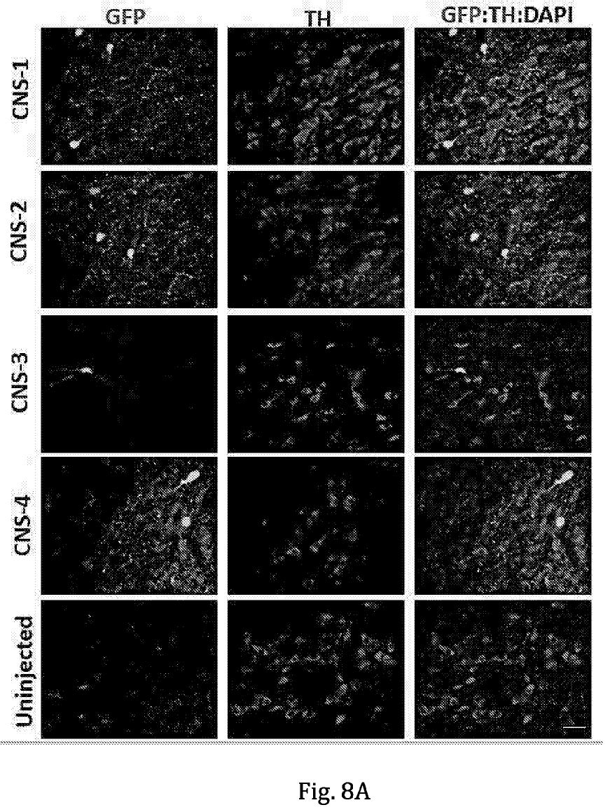

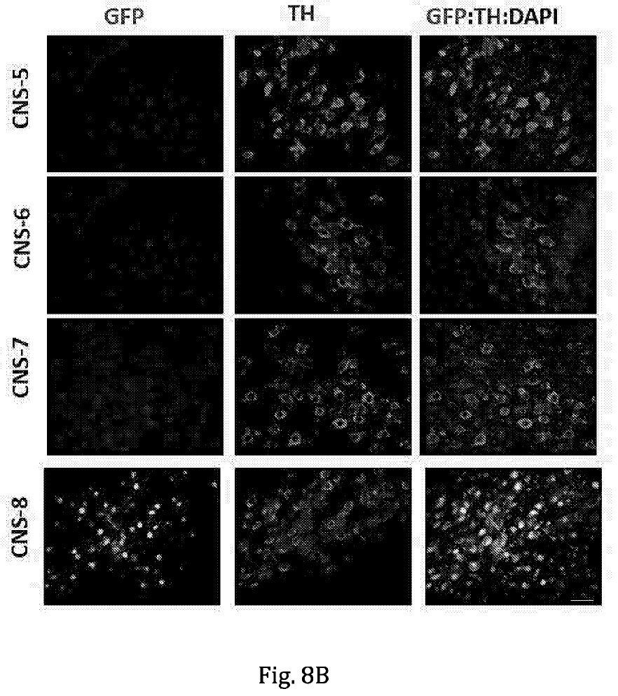

In ICV delivery, CNS-1 (SEQ ID NO: 1) and CNS-2 (SEQ ID NO: 2) had very high percentage GFP immunoreactivity in the midbrain, followed by CNS-7 (SEQ ID NO: 25), CNS-5 (SEQ ID NO: 23) and CNS-4 (SEQ ID NO: 4) with the rest of the tested promoters showing very little or no GFP immunoreactivity in the cortex. In IV delivery, CNS-1-8 (SEQ ID NO: 1-4,23-26) showed very little or no GFP immunoreactivity in the midbrain while the control promoter Syn-1 (SEQ ID NO: 14) showed very high GFP immunoreactivity in the midbrain. Notably, CNS-8 (SEQ ID NO: 26) in IV delivery showed activity in dopaminergic neurones in the midbrain as shown in FIG. 8B even though it shows very low GFP immunoreactivity in the midbrain.

In ICV delivery, CNS-1-8 (SEQ ID NO: 1-4,23-26) showed mid or low percentage GFP immunoreactivity in the cerebellum. The percentage GFP immunoreactivity of CNS-1 (SEQ ID NO: 1) and CNS-5-8 (SEQ ID NO: 23-26) was higher than the percentage GFP immunoreactivity of the control promoter Syn 1(SEQ ID NO: 14). In IV delivery, the control promoter Syn-1 (SEQ ID NO: 14) had high percentage GFP immunoreactivity in the cerebellum but the rest of the tested promoters had very little or no GFP immunoreactivity in the cerebellum.

Notably, CNS-1 (SEQ ID NO: 1) had high or mid percentage GFP immunoreactivity per area in all tested brain areas in ICV delivery. Similarly, CNS-2 (SEQ ID NO: 2) had high percentage GFP immunoreactivity per area in four out of the five tested areas (apart from cerebellum) in ICV delivery.CNS-8 (SEQ ID NO: 26) had very low or no percentage GFP immunoreactivity per area in all tested brain areas but still showed expression in dopaminergic neurones.

### Example 2

The biodistribution of the transgene GFP under the control of CNS-8 (SEQ ID NO: 26) was further investigated at a higher dose in IV and ICV delivery (herein called the high dose). The intercranial and intravenous injections were performed as described in Example 1, but 5×10¹¹ viral genomes/pup were injected in intracranial injections and 2×10¹² vg/pup were injected in intravenous injections (10-fold higher dose). The dose used in IV and ICV delivery in Example 1 is herein called the low dose.

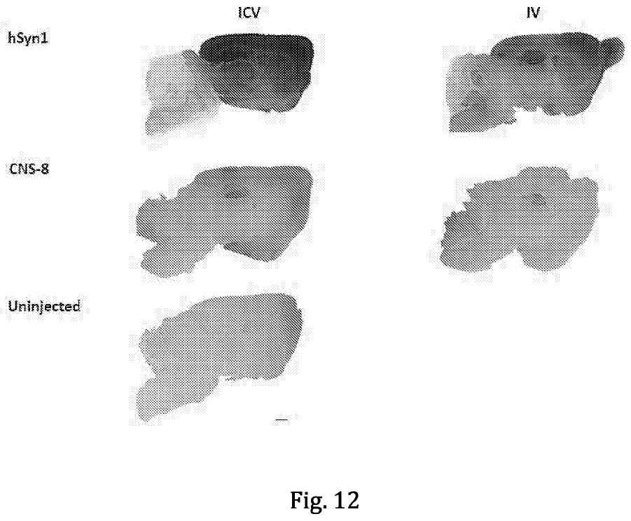

The biodistribution of GFP under the control of CNS-8 when low dose was administered (Example 1) was very similar to the biodistribution of GFP under the control of CNS-8 when high dose was administered (Example 2) in sagittal sections in both ICV and IV delivery, as shown in FIG. 2B and FIG. 12.

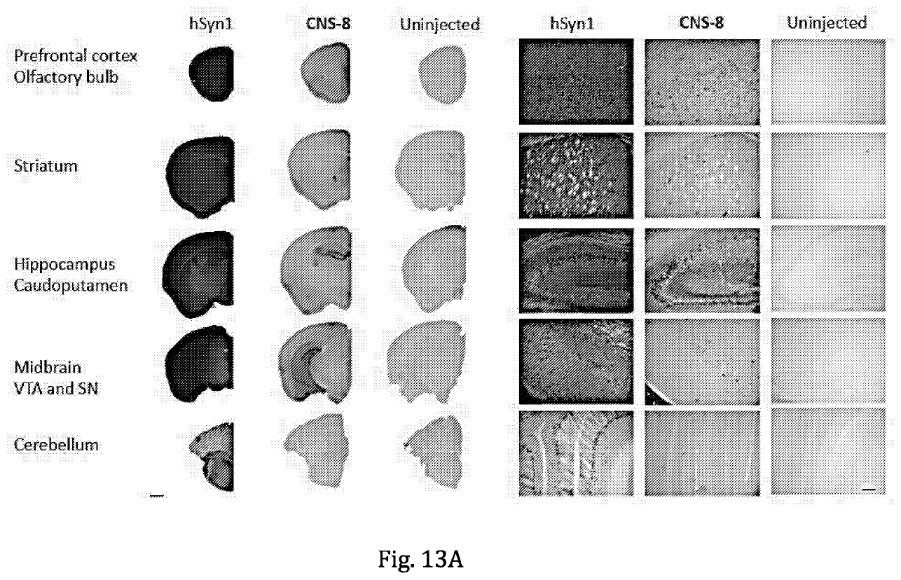

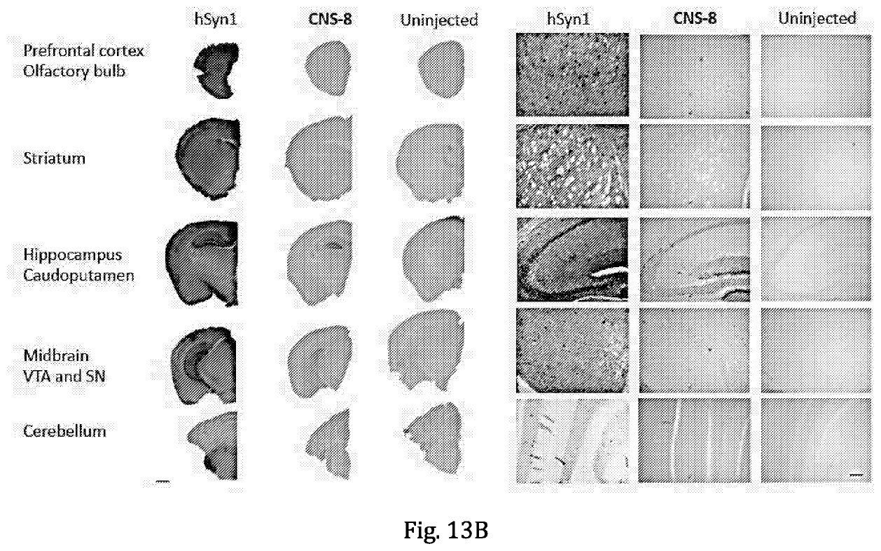

Similarly, the biodistribution of GFP under the control of CNS-8 (SEQ ID NO: 26) when low dose was administered was very similar to the biodistribution of GFP under the control of CNS-8 when high dose was administered in coronal sections in ICV delivery at higher and lower magnification, as shown in FIG. 3B, FIG. 5B and FIG. 13A.

The biodistribution of GFP under the control of CNS-8 (SEQ ID NO: 26) when low dose was administered was very similar to the biodistribution of GFP under the control of CNS-8 when high dose was administered in coronal sections in IV delivery at higher and lower magnification, as shown in FIG. 4B, FIG. 6B and FIG. 13B.

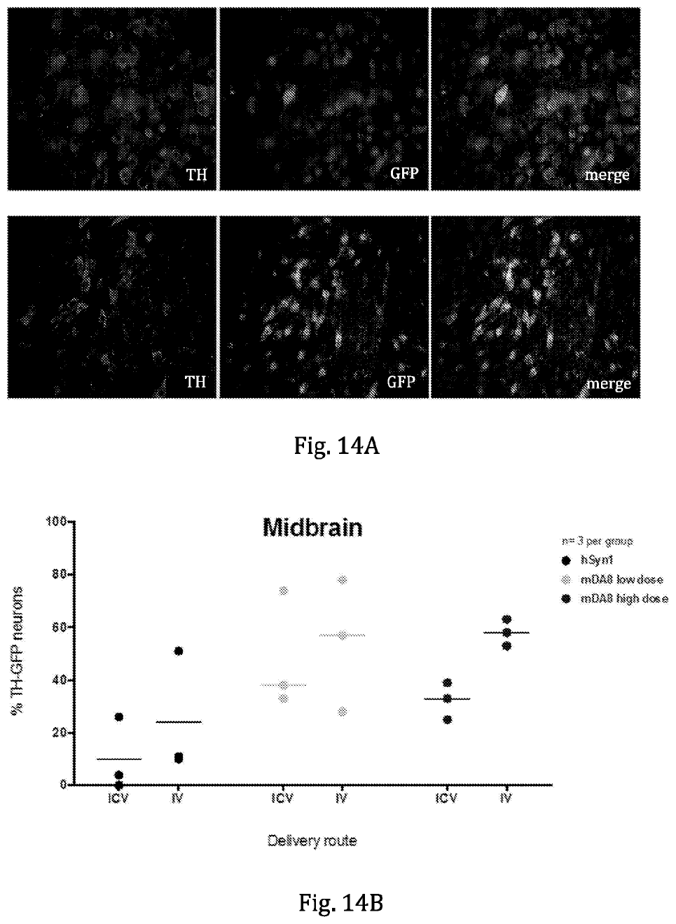

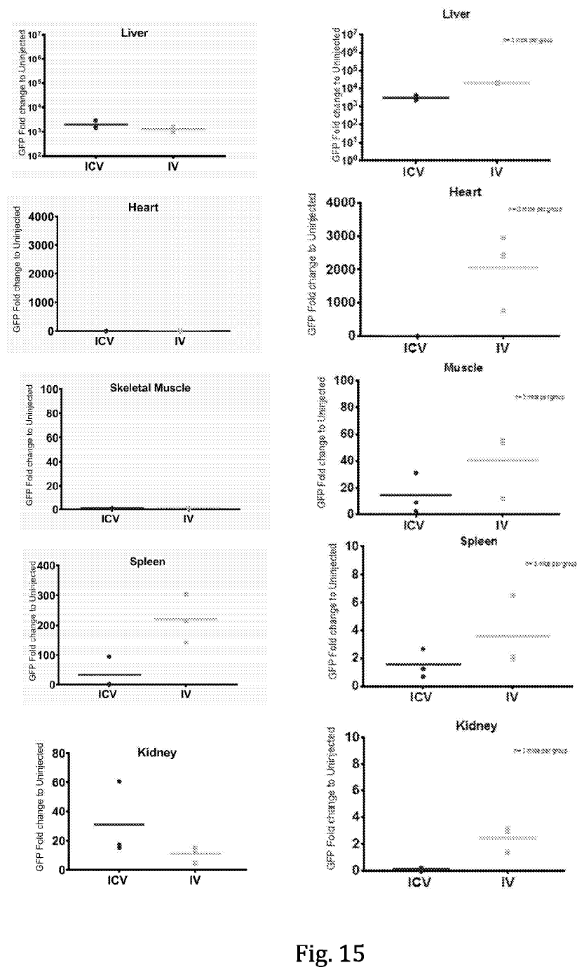

Similarly, the GFP expression under the control of CNS-8 (SEQ ID NO: 26) in the midbrain when low dose was administered was very similar to the GFP expression under the control of CNS-8 (SEQ ID NO: 26) when high dose was administered in both ICV and IV delivery as shown in FIG. 7B, FIG. 8B and FIG. 14A. This was supported by quantification of the GFP positive dopaminergic neurones which showed that there was no difference in the percentage of the GFP positive dopaminergic neurones between the low and high dose administration in both ICV and IV delivery, as shown in FIG. 14B. Therefore, there was no overall difference in the GFP expression under the control of CNS-8 promoter (SEQ ID NO: 26) between low and high dose administration indicating that the low dose is sufficient to show GFP expression in dopaminergic neurones and that increase in the dose does not result in higher GFP expression. Suitably, the lowest dose which shows the required expression pattern may be preferable.

Comparison of the biodistribution in different tissues of the transgene GFP under the control of CNS- 8 (SEQ ID NO: 26) when a low or a high dose was administered revealed that the dose influenced the GFP expression. In liver, the GFP expression was very similar between doses in ICV delivery but lower in the low dose in IV delivery. In hearth, no GFP expression was detected in the low dose in either ICV or IV delivery while in the high dose, GFP expression was detected in IV delivery. Similarly, in skeletal muscle, no GFP expression was detected in the low dose in either ICV or IV delivery while in the high dose, GFP expression was detected in both ICV and IV delivery. However, in spleen, higher GFP expression was detected in the low dose in both IV and ICV delivery compared to the high dose. Similarly, in kidney, higher GFP expression was detected in the low dose in both IV and ICV delivery compared to the high dose. This data indicates that administration of different doses of viral genomes may result in different expression patterns and expression levels in tissues other than the CNS.

Therefore, varying the dose did not change the GFP expression and level in the CNS but changed the expression patterns and expression levels in tissues other than the CNS. Therefore, it may be possible to find an optimum dose depending on the expression pattern and level requirements in tissues other than the CNS while maintaining the expression pattern and level in the CNS. For example, if an activity in the CNS as well as activity in the liver, spleen and kidney was required, the low dose may be administered via ICV or IV. Alternatively, if activity in the CNS as well as activity in at least the heath and the skeletal muscle was required, then the high dose may be administered via IV delivery.

### Example 3

The tissue expression pattern for the faf1 and pitx3 genes from which the CRE/proximal promoter from CNS-5, CNS-5_v2, CNS-2, CNS-3 and CNS-4 were designed was investigated in a single-cell transcriptomic dataset (Zeisel et al., 2018). Due to the proximity of the CRE/proximal promoter to the gene, it is expected that the CRE/proximal promoter assists in the regulation of the gene (He et al., 2014). Providing the CRE/proximal promoter regulates the expression of their nearest gene, the expression patterns for the gene provides an indication of the possible expression profile of a synthetic promoter comprising the CRE/proximal promoter.

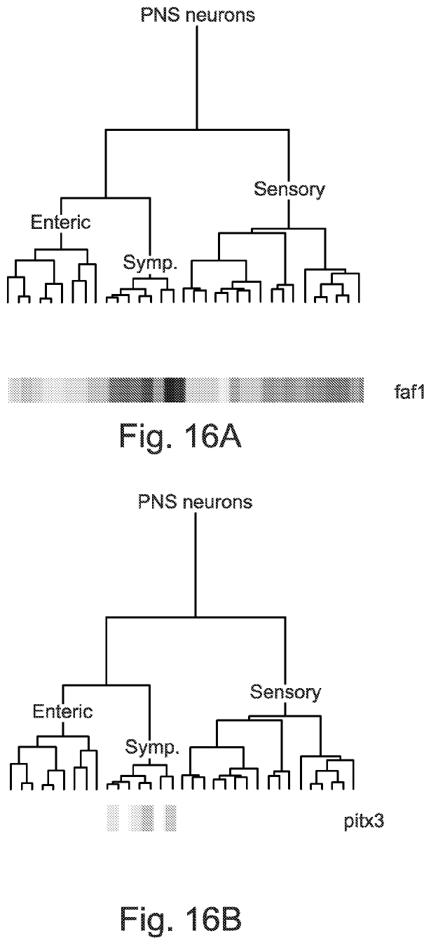

The single-cell transcriptomic dataset (Zeisel et al., 2018) contains single-cell RNA sequencing of 500 000 cells of any type from the CNS and PNS of an adult mouse. The resource is publicly available at mousebrain.org/genesearch.html and provides a useful tool to determine the possible expression of synthetic promoters CNS-5, CNS-5_v2, CNS-2, CNS-3 and CNS-4 in the PNS. The genes from which the CRE/proximal promoter of CNS-5, CNS-5_v2, CNS-2, CNS-3 and CNS-4 were designed were added to the webtool and the expression pattern of faf1 and pitx3 gene is displayed in FIG. 16A and 16B. The gradient of grey indicates the strength of RNA expression detected in the database (Zeisel et al., 2018). faf1 is expressed in many PNS neurones so a synthetic promoter comprising CRE or proximal promoter designed from the faf1 gene such as CNS-5 and CNS-5_v2 is expected to have strong expression in the PNS. pitx3 is expressed in sympathetic PNS neurones so a synthetic promoter comprising CRE designed from the pitx3 gene such as CNS-2, CNS-3 or CNS-4 is expected to have expression in PNS sympathetic neurones. Similar analysis for Imx1b and pitx2 revealed no expression in PNS above the cut off score for the analysis (trinization score of less than 0.95; data not shown) so CNS-1, CNS-6, CNS-6_v2, CNS-7, CNS-7_v2, CNS-8 and CNS-8_v2 are not expected to be active in PNS neurones.

### References

Boussicault, L. et al. (2016) ‘CYP46A1, the rate-limiting enzyme for cholesterol degradation, is neuroprotective in Huntington's disease’, Brain, 139(3), pp. 953-970. doi: 10.1093/brain/awv384.

Djelti, F. et al. (2015) ‘CYP46A1 inhibition, brain cholesterol accumulation and neurodegeneration pave the way for Alzheimer's disease’, Brain, 138(8), pp. 2383-2398. doi: 10.1093/brain/awv166.

Hammond, S. L. et al. (2017) ‘Cellular selectivity of AAV serotypes for gene delivery in neurons and astrocytes by neonatal intracerebroventricular injection’, PLoS ONE, 12(12), pp. 1-22. doi: 10.1371/journal.pone.0188830.

He, B. et al. (2014) ‘Global view of enhancer-promoter interactome in human cells’, Proceedings of the National Academy of Sciences of the United States of America, 111(21). doi: 10.1073/pnas.1320308111.

Jakobsson, J. and Lundberg, C. (2006) ‘Lentiviral vectors for use in the central nervous system’, Molecular Therapy. The American Society of Gene Therapy, 13(3), pp. 484-493. doi: 10.1016/j.ymthe.2005.11.012.

Kacher, R. et al. (2019) ‘CYP46A1 gene therapy deciphers the role of brain cholesterol metabolism in Huntington's disease’, Brain: a journal of neurology, 142(8), pp. 2432-2450. doi: 10.1093/brain/awz174.

Kim, Ji-Yoen; Ash, Ryan T.; CAballos-Diaz; CArolina, Levites, Yona; Golde, Todd E.; Smirnakis, Stelios M.; Jankowsky, J. L. (2013) ‘Viral transduction of the neonatal brain delivers controllable genetic mosaicism for visualizing and manipulating neuronal circuits in vivo’, European Journal of Neuroscience, 37(8), pp. 1203-1220. doi: 10.1111/ejn.12126.Viral.

Tanguy, Y. et al. (2015) ‘Systemic AAVrh10 provides higher transgene expression than AAV9 in the brain and the spinal cord of neonatal mice’, Frontiers in Molecular Neuroscience, 8(JULY), pp. 1-10. doi: 10.3389/fnmo1.2015.00036.

Zeisel, A. et al. (2018) ‘Molecular Architecture of the Mouse Nervous System’, Cell, 174(4), p. 999-1014.e22. doi: 10.1016/j.ce11.2018.06.021.

Sequence Information

TABLE 1
CNS-specific promoters

| NAME | SEQUENCE | Length |
|---|---|---|
| CNS-1 | CTGGGCAGAGAGGGGGCATCGGGGGCATGGCTAGGGGCCAGCACTGTGCTTCCTGGGCGCCTC | 696 |
| (SEQ ID | ACCTCCTCCCTGACTCCTGGAGACTCCCAGCCCCTGTCTGGGAGATGAGCATTTAGGAATCTG | |
| NO: 1) | CTTGTGCAGGGGTGGTGGGAGGGGCCGGGGTGGAGGGCGCATCCCCACGGGGAGATTGGATGG | |
| | AAATGGCCTGCCAGTGTGTGTGTGAGTGTGCGCCTGTGGCAGCAGCAGAGTAAACAGCCGCTG | |
| | CCCTGTCCTCTCTGCGGCCGTGGCCAGGTACACAGGCCTGTTTGGACAGCTGCCTTGTCTGTC | |
| | CGTCTGTTTGGGAGATGCTGGCTGATAGATGGGGATGGGCGGACTGTTAACCCCTCGTTGCCT | |
| | GCACTGCTATGTGCTTCCTGCCTCATCCATGGGGTAGAAGGTAGCCAGAAGGTGGTCCTGGCT | |
| | GTGCCCCCAGCTCCTCTCTAGGGGGGAAACCTCTAGTTCTGAGTCAGGGACAGAGTGAGGAGG | |
| | GCTCCAGGGCATCAAGAGCTTGCTCCTCCCCGCACCAGGGAGCCAAGGACAGAGGAGAAGGGG | |
| | GTCTTCCCCAGTGGTGACTAGGGGCAGAATATGTCTCTGAGTGAGTGTCTGGAGCCCTCCTCA | |
| | CCCCAACACCATGGGGCTGGGCATAAAAGTCAGGGCAGAGCCATCTATTGCTTACATTTGCTT | |
| | CTG | |
| CNS-2 | GGTGTGTGGAAGGGTGAGAGGCACACACACAGACACTGAAAGAATCCTAGGCCTGGTAGGCAC | 709 |
| (SEQ ID | TTAACAAATGTCTGTTACAGACCAGAATTTTATTGCTGTTAGAGACCCAAGCCCCTCATAGGA | |
| NO: 2) | ACAGTGAGAAACAGGTGCAGAAAGGCGGAGTAACTTTATCTAAAGTCATAGGCTCCCTGAATA | |
| | GCAGAGCTGACACCTACAAGGAAGCGTTGGAGACCAGATCTACCAGCTAGCCTCCCTGAGACC | |
| | ACGAGGTGGCGCCGCAGCACCGGCTGTGGCCGATGCCAGCCAGGTAGCCGGTTTCCCACGTCC | |
| | CCCGCACGCACGCACCTCTTTGCTGCAGGAATCCCGGGCTGCCCCGACCTGGAGTAGGGGGGG | |
| | TGGTGAGTGGGACTGAGTCCCTAGAAGCCTGGACCCTCACTTCGTTCCTGTACATCCAGCTCG | |
| | CCTGTAGACAGTGGGGGAGGATGAAGGGAAGAGGACTCAAGCGCAACTTTGAATCATCACGCC | |
| | TTCGACAGTCCGCGCACGTTTATTTCATTTATCTTTGAAAACGAGGGAGGGGAAGCCTGGAGA | |
| | AGGCGGGATGGGCCAAGGGTGAGTTGGCCCCCGGGGAGCTGGTCCCTGTTCCTGGCTTTAGTC | |
| | CCAGGGGCGCGGTCTGTGTGTAGGGCGGGCTGGGCATAAAAGTCAGGGCAGAGCCATCTATTG | |
| | CTTACATTTGCTTCTG | |
| CNS-3 | TGCTACCAGAGCCGGGAGAGCTGCTCGGAGACGCCTCCGGGGTGCGGGCTGGACATGAGCAGC | |
| (SEQ ID | GGCTGCCGGTCCTGGGACTAGGCCCCGCCATTTTGGATCCGCTGACAGGTCAGCGAAGTCTCT | |
| NO: 3) | TCCTAGAGTTCCGGTGTCGTGAAGGCCGCCCTGACATCGCAATAGGGAATTAGTGGGAAGGGC | |
| | CCTTAAATTGGGCGAGCCAAGGTGGGGGGAGGATTGGAACAGAGACAAAAGGGAGGAGAGACG | |
| | GACAGCGACAAGTGGAGAAAATCGGCGAAACTTGAGTGGCAGAGAAGTCTGAGCGCTGAGACC | |
| | CGGCGGCCCCGTGCGCCTTCCCACCTGGCGCCGATCCACTTTCCTCGGGGTAGCGGCCCAACC | |
| | CACTTCGCTGCCAGCCGATCCCTTTTACCCGTGGCTACCGGGACCACTCTACTCTCGCCCACT | |
| | TGGCTCTGCCTAAGCGTCCTAGCCGGAGCGCGGTCTCTGCCACGTGGGGAGGGGCGCGGCCGA | |
| | GTTGCTGAAGAGCGCTTCTGATTGGCCAGAGGGCGGGGTTCTTGGCGTCTCGCCGGCCAGACC | |
| | CCTCCCTCAAAGGCGGGGCCTGGAGATCCACAGCTGGAAAGGGCGGAGCCCCAGCAGGGCAGC | |
| | TGGAAAGGGGCGGGGCCTGACGCGCGCGGCTCGCCGCGGCGGGCTGGGGGCGCCCTGGTCTGC | |
| | CATAAAGTGAATGGGCGCCGGCTGGGGGTGGCAGTACGCGGTGAGGCTCACTCCCTCCGAGAG | |
| | TCCAGGAGCGCC | |
| CNS-4 | AAGGAGAATGGTAAACAGCAGGAGCGAAGCGGCTGAGGAGAAAGAAGAGGAAAGAAAGGCGAG | |
| (SEQ ID | ACGTGGGAGGATTGGAACAGAGACAAAAGGGAGGAGAGACGGACAGCGACAAGTGGAGAAAAT | |
| NO: 4) | CGGCGAAACTTGAGTGGCAGAGAAGTCTGAGCGCTGAGACCCGGCGGCCCCGTGCGCCTTCCC | |
| | ACCTGGCGCCGATCCACTTTCCTCGGGGTAGCGGCCCAACCCACTTCGCTGCCAGCCGATCCC | |
| | TTTTACCCGTGGCTACCGGGACCACTCTACTCTCGCCCACTTGGCTCTGCCTAAGCGTCCTAG | |
| | CCGGAGCGCGGTCTCTGCCACGTGGGGAGGGGCGCGGCCGAGTTGCTGAAGAGCGCTTCTGAT | |
| | TGGCCAGAGGGCGGGGTTCTTGGCGTCTCGCCGGCCAGACCCCTCCCTCAAAGGCGGGGCCTG | |
| | GAGATCCACAGCTGGAAAGGGCGGAGCCCCAGCAGGGCAGCTGGAAAGGGGCGGGGCCTGACG | |
| | CGCGCGGCTCGCCGCGGCGGGCTGGGGGCGCCCTGGTCTGCCATAAAGTGAATGGGCGCCGGC | |
| | TGGGGGTGGCAGTACGCGGTGAGGCTCACTCCCTCCGAGAGTCCAGGAGCGCCCGAGCGGAGA | |
| | GGCGGCCCGGGAGCAGGGGGGCGGCCCCCACTCCGGCCGGGTGCCCGGCCCCTGGCCCCTGCC | |
| | TGCCCTCTAGATCGCCGCCGCAGCCGCCGCTACTGGGAGTCTG | |
| CNS- | ttgtAATGGGAATAAGGGCAGGACtcctgggtataagtagctcagctgatcccaccctgcttc | 1744 |
| 5_v2 | tatgtgttaattcatttattcattcattcaacaagcatttgttgaaatgctctttgtgtcagg | |
| (SEQ ID | ctcagcaggaagcagtggcaataaaatggtgaacaagaaagactcggggtttcttcatctatg | |
| NO: 5) | ttgatgtctgcagagaacagtatcagccttctaggaagtttgtaatcagatacattgttagag | |
| | agatacttatctagtaaattcctactcatcctataaggctcaaaacaaatgcctctatgaaac | |
| | cttccgtgattccctcaggcagagttaagagcttcctttcctgggcctctatctccttccatt | |
| | agtattataactgtttaccagtttcccctctagactaaatttctcaaaagagagaatgaggtc | |
| | tctttcagtcttctttgcatctttaaactagcctgggtcccagcctgtttgatgaaagaaaca | |
| | agaacactgatacaagccacagccccttggcaaaaaagatacccaatagcaatggcaatgtaa | |
| | aatcagttttagtaaatgaatcaagaattctgatgctttagggaaagtaatgtgaacctggca | |
| | ccattaacaaattcagaactcttcttcttaggagctctctaactgaacagacagagggatgtc | |
| | aacccctaattcagcttgatcgtatctcagcaactacatttaatgagacagtgggaaaaagag | |
| | agctgtccacttttaaatcagcatatttctaactaaacaatggcaatggctaaatctttaaaa | |
| | tgcctatttctctcaagaacactgcaatggaacatttagactttgggaaagagattagtgatt | |
| | tacattgctatctcactgatttaatttaaatgctcttccaaaccaaacacacatgtgccgaag | |
| | aggctactaagaaacccaacatgcagagttctctataagtgcagccgacagtgttgactgaaa | |
| | ctaaacttggaaatccagggcactaatgcacaatatcaagcaataaaacggcatctctttggc | |
| | aatatttaatttaaaaaagaagaaagagacaggcgaagatcaggcactgtctgttttggagga | |
| | tcaaccattctgcatttcaaagcattggtccctgcaatatccaggttactgtgctagaatctc | |
| | gactattatatcgcagttgtgagagggagggcaaagatgtgtttactcagtgattaggccctt | |
| | agaataagcctctagctcctagagagacagctcaccacttattcatttgggccaattcacaaa | |
| | gcctaggaagattaaacatccatgctgagaagacaagcgaatgcagacggtgaaaaagaaata | |
| | aaaattctttaaaaactctgagatgacttcattatttttccacaaggaaactttaggaaagtg | |
| | tttagttagagaaaaacccacattgacctctctctaaacccttaatctttcctttgtggtggc | |
| | actgctttgtggtaagcgactggctcgcctcgcccctcttttcactggaagctgagagaaaaa | |
| | agactctggagaaacagttttcgttccagggacacaaacccctgacactgttaaacatgagat | |
| | gccaggaaaacacacttaaaaaaaaaacccactttaagctttagactgaatgtgagaaaggag | |
| | atgataaaaagagtatcacaaGAGAATCTTCAGGCTGTGGG | |
| CNS- | TTTGGCACTGTGAGCAGTTTACttgacaaattctgtcaaatatttgctttctgaaatctcgag | 1104 |
| 6_v2 | aattggttgaatataattgtacttaatgtttgcaaaataaataaatatgggactaaggacgtt | |
| (SEQ ID | ctatcattaatttgtcagaaaagagagttgtcatttctgaaaatttaatgtcattgaagctct | |
| NO: 6) | atttccaatagcaaaggagcactattgctaatagacttcagagcttgaaataaataaatcttt | |
| | ggaatcctgttgcatctcttggggtgtgacatttgacagtcttttatagcacagaacgaaaca | |
| | agtttgtgagctggaattcaattgtggcgtattgattccttgcatcagtcattattccctgct | |
| | gattgacaggtgaaaattggttacgttaagtatttcatatgttatattggctgacatttgctt | |
| | gcctgctcttgtgtcaatattgttgtaaagatctccagctttatgagatagcaatagacactg | |
| | actgtggcttttgtgtgatgttccagtgtttttcctgacataatttaagacatattaaaaacc | |
| | agcagcatcttccctcttgagaagcttaatgccaatattattgtcttccaggggaagatcatg | |
| | tatgctcataatcgggtgctaatttccaccagtacgctcatgtttaggcattaggcactataa | |
| | ctgtaaaattgagccttcttgattgattcatgtcaagcctcatctcggctcctgcaggggaag | |
| | tcatccggctgaccctttttacactaaaagaagagatttgtgttcctttctttcacctggaac | |
| | catcaaattgactgaataatctgtaatacattagtgctgacatttgttagggagaattaaaca | |
| | agacacagtaatcattccccagaataaaaattgtgtttgatttccagcagagttctattaaag | |
| | ggaggacagaatctgtctcttccaaggtggaaaatcgtgaatattccctgcattaatgaacca | |
| | agttaacactttaattgcttatagaaccgagttctccaatgacagcattaaaagatagggagg | |
| | ctctgatttaTGGTCAACACAGATTTGTAACCC | |
| CNS- | TCAACATGGATAACCAAAGTTCTtaaaactacgctttcaatgaacacatatcctttgagcaag | 1941 |
| 7_v2 | actaataatgaggaatgggagccagctcctgtgatatttatgcaactactaaattctcactga | |
| (SEQ ID | agtcaatgggagtttgcttacgtaagggctgcaaactttagcctccagagattaaaggggaaa | |
| NO: 7) | aaaatccttaaactctttcaacattaatattgcctgtaaggaatccagccatgacctaagcca | |
| | tggagctttctgaacctagcaagtagaagggtaaacagtaaacaccagttattttaagcacaa | |
| | tctaatcagagttcaatgagaagcaatattatatttgatctctaaggtattaatacttgtata | |
| | tcactattagacatctttatgtagtccattatccaaacaatggcttaagtctgtggtatttaa | |
| | taaatcaagtttccatggccgtgagactgagtgggagtggggatgaagccttttttcttcatt | |
| | tttttttcctcaggtgcaattctgtgttaatataagagaagtgtggccttccttctcatagca | |
| | ctaaaagtgagataatccctgtgtaagaaatcagtaagtacggtctgcttaatctagtcccag | |
| | tgtgaaactgttgacatttgttcttttttctatcattatgtgactgggcctgttttgtgctgg | |
| | attaggcacaaatctcctatgcagcacatttggcatgttactagtagtttaacttcattaata | |
| | atgtatgaagaaaatgtaatccatgacaaggaagcaaagaaaagtatttttttttttttttgc | |
| | ttctcccaaatcctttggaatgagtaattattcaacattttatgtttgatgttatattttaca | |
| | attcaacttccatagtgatatttaaaaaagaaactttggcaaatgcttgcaaaaaacacacct | |
| | tttacaattttaaatgtgatttactgatggccagaacttgttaaacatagtaggaaattaaat | |
| | atttattcatcttatttcattttcagggccgtaaacgctccttctgagtcattcccaataaca | |
| | agaatttctaccagtaaagctattaacaggcatcaaaataggggagtgctaaattaagatgag | |
| | attgtaaaagcaaataagaacatacgcagactcgcataggagtgcaaatgatcgtttctgatt | |
| | gaaatgtttatagctaaatgagtttggctgaattaaacacaaatgttccaaaagataagccgt | |
| | agctggtgcttcttttttctgttttttaagctgctttacagacgaaaatggaactatatttgg | |
| | aacaatgctttctgtttttccatactattgatatttgtggaaagtcacaaaatggcctaagga | |
| | agctaagctcgccccaagcagtggtcacttacaagtacttttgtactctgtactcctgtcaca | |
| | tttgggcgatcagagcaacagctggggagactttttcaacaaagatgagtgtcagataatcct | |
| | gatgagattccacatccaacatcttttgtaattatgtcacattcagctgtaatggaataattc | |
| | aagctgaaagaacaagctttgatcctttcttaaacctttccctgtggactggctatctaaaag | |
| | atttaaagatatttctgttacaagatctagtgtttcctcagagaagtcatgcttctgaagcat | |
| | cgtgatctacaagaacaatatcaagtttgccaaacacatttctgaaagcatcgtgttttgggg | |
| | ggaggggttgtatttaatgaagatatcaataatatgctatgcttcaattttcatctaggtgat | |
| | caagattcattttcttgttctgtcatccaaataggcagacagaaaagtgattgaaatacatta | |
| | TGGAGATGTGTCATTGCACA | |
| CNS- | GCTGGTGCTTCTTTTTTCTGTTTTTTAAGCTGCTTTACAGACGAAAATGGAACTATATTTGGA | 540 |
| 8_v2 | ACAATGCTTTCTGTTTTTCCATACTATTGATATTTGTGGAAAGTCACAAAATGGCCTAAGGAA | |
| (SEQ ID | GCTAAGCTCGCCCCAAGCAGTGGTCACTTACAAGTACTTTTGTACTCTGTACTCCTGTCACAT | |
| NO: 8) | TTGGGCGATCAGAGCAACAGCTGGGGAGACTTTTTCAACAAAGATGAGTGTCAGATAATCCTG | |
| | ATGAGATTCCACATCCAACATCTTTTGTAATTATGTCACATTCAGCTGTAATGGAATAATTCA | |
| | AGCTGAAAGAACAAGCTTTGATCCTTTCTTAAACCTTTCCCTGTGGACTGGCTATCTAAAAGA | |
| | TTTAAAGATATTTCTGTTACAAGATCTAGTGTTTCCTCAGAGAAGTCATGCTTCTGAAGCATC | |
| | GTGATCTACAAGAACAATATCAAGTTTGCCAAACACATTTCTGAAAGCATCGTGTTTTGGGGG | |
| | GAGGGGTTGTATTTAATGAAGATATCAATAATATGC | |
| CNS-1 + | CTGGGCAGAGAGGGGGCATCGGGGGCATGGCTAGGGGCCAGCACTGTGCTTCCTGGGCGCCTC | 982 |
| CMV-IE | ACCTCCTCCCTGACTCCTGGAGACTCCCAGCCCCTGTCTGGGAGATGAGCATTTAGGAATCTG | |
| UTR | CTTGTGCAGGGGTGGTGGGAGGGGCCGGGGTGGAGGGCGCATCCCCACGGGGAGATTGGATGG | |
| and | AAATGGCCTGCCAGTGTGTGTGTGAGTGTGCGCCTGTGGCAGCAGCAGAGTAAACAGCCGCTG | |
| intron | CCCTGTCCTCTCTGCGGCCGTGGCCAGGTACACAGGCCTGTTTGGACAGCTGCCTTGTCTGTC | |
| (SEQ ID | CGTCTGTTTGGGAGATGCTGGCTGATAGATGGGGATGGGCGGACTGTTAACCCCTCGTTGCCT | |
| NO: 21) | GCACTGCTATGTGCTTCCTGCCTCATCCATGGGGTAGAAGGTAGCCAGAAGGTGGTCCTGGCT | |
| | GTGCCCCCAGCTCCTCTCTAGGGGGGAAACCTCTAGTTCTGAGTCAGGGACAGAGTGAGGAGG | |
| | GCTCCAGGGCATCAAGAGCTTGCTCCTCCCCGCACCAGGGAGCCAAGGACAGAGGAGAAGGGG | |
| | GTCTTCCCCAGTGGTGACTAGGGGCAGAATATGTCTCTGAGTGAGTGTCTGGAGCCCTCCTCA | |
| | CCCCAACACCATGGGGCTGGGCATAAAAGTCAGGGCAGAGCCATCTATTGCTTACATTTGTCA | |
| | GATCGCCTGGAGACGCCATCCACGCTGTTTTGACCTCCATAGAAGACACCGGGACCGATCCAG | |
| | CCTCCGCGGCCGGGAACGGTGCATTGGAACGCGGATTCCCCGTGCCAAGAGTGACGTAAGTAC | |
| | CGCCTATAGACTCTATAGGCACACCCCTTTGGCTCTTATGCATGAACGGTGGAGGGCAGTGTA | |
| | GTCTGAGCAGTACTCGTTGCTGCCGCGCGCGCCACCAGACATAATAGCTGACAGACTAACAGA | |
| | CTGTTCCTTTCCATGGGTCTTTTCTGCAGATGCCACC | |
| CNS-4 + | AAGGAGAATGGTAAACAGCAGGAGCGAAGCGGCTGAGGAGAAAGAAGAGGAAAGAAAGGCGAG | 876 |
| CMV-IE | ACGTGGGAGGATTGGAACAGAGACAAAAGGGAGGAGAGACGGACAGCGACAAGTGGAGAAAAT | |
| UTR | CGGCGAAACTTGAGTGGCAGAGAAGTCTGAGCGCTGAGACCCGGCGGCCCCGTGCGCCTTCCC | |
| and | ACCTGGCGCCGATCCACTTTCCTCGGGGTAGCGGCCCAACCCACTTCGCTGCCAGCCGATCCC | |
| intron | TTTTACCCGTGGCTACCGGGACCACTCTACTCTCGCCCACTTGGCTCTGCCTAAGCGTCCTAG | |
| (SEQ ID | CCGGAGCGCGGTCTCTGCCACGTGGGGAGGGGCGCGGCCGAGTTGCTGAAGAGCGCTTCTGAT | |
| NO: 22) | TGGCCAGAGGGCGGGGTTTTGGCGTCTCGCCGGCCAGACCCCTCCCTCAAAGGCGGGGCCTGG | |
| | AGATCCACAGCTGGAAAGGGCGGAGCCCCAGCAGGGCAGCTGGAAAGGGGCGGGGCCTGACGC | |
| | GCGCGGCTCGCCGCGGCGGGCTGGGGGCGCCCTGGTCTGCCATAAAGTGAATGGGCGCCGGCT | |
| | GGGGGTGGCAGTACGCTCAGATCGCCTGGAGACGCCATCCACGCTGTTTTGACCTCCATAGAA | |
| | GACACCGGGACCGATCCAGCCTCCGCGGCCGGGAACGGTGCATTGGAACGCGGATTCCCCGTG | |
| | CCAAGAGTGACGTAAGTACCGCCTATAGACTCTATAGGCACACCCCTTTGGCTCTTATGCATG | |
| | AACGGTGGAGGGCAGTGTAGTCTGAGCAGTACTCGTTGCTGCCGCGCGCGCCACCAGACATAA | |
| | TAGCTGACAGACTAACAGACTGTTCCTTTCCATGGGTCTTTTCTGCAGATGCCACC | |
| CNS-5 | GGAACATTTAGACTTTGGGAAAGAGATTAGTGATTTACATTGCTATCTCACTGATTTAATTTA | 835 |
| (SEQ ID | AATGCTCTTCCAAACCAAACACACATGTGCCGAAGAGGCTACTAAGAAACCCAACATGCAGAG | |
| NO: 23) | TTCTCTATAAGTGCAGCCGACAGTGTTGACTGAAACTAAACTTGGAAATCCAGGGCACTAATG | |
| | CACAATATCAAGCAATAAAACGGCATCTCTTTGGCAATATTTAATTTAAAAAAGAAGAAAGAG | |
| | ACAGGCGAAGATCAGGCACTGTCTGTTTTGGAGGATCAACCATTCTGCATTTCAAAGCATTGG | |
| | TCCCTGCAATATCCAGGTTACTGTGCTAGAATCTCGACTATTATATCGCAGTTGTGAGAGGGA | |
| | GGGCAAAGATGTGTTTACTCAGTGATTAGGCCCTTAGAATAAGCCTCTAGCTCCTAGAGAGAC | |
| | AGCTCACCACTTATTCATTTGGGCCAATTCACAAAGCCTAGGAAGATTAAACATCCATGCTGA | |
| | GAAGACAAGCGAATGCAGACGGTGAAAAAGAAATAAAAATTCTTTAAAAACTCTGAGATGACT | |
| | TCATTATTTTTCCACAAGGAAACTTTAGGAAAGTGTTTAGTTAGAGAAAAACCCACATTGACC | |
| | TCTCTCTAAACCCTTAATCTTTCCTTTGTGGTGGCACTGCTTTGTGGTAAGCGACTGGCTCGC | |
| | CTCGCCCCTCTTTTCACTGGAAGCTGAGAGAAAAAAGACTCTGGAGAAACAGTTTTCGTTCCA | |
| | GGGACACAAACCCCTGACACTGTTAAGGGCTGGGCATAAAAGTCAGGGCAGAGCCATCTATTG | |
| | CTTACATTTGCTTCTG | |
| CNS-6 | GAAAATTTAATGTCATTGAAGCTCTATTTCCAATAGCAAAGGAGCACTATTGCTAATAGACTT | 812 |
| (SEQ ID | CAGAGCTTGAAATAAATAAATCTTTGGAATCCTGTTGCATCTCTTGGGGTGTGACATTTGACA | |
| NO: 24) | GTCTTTTATAGCACAGAACGAAACAAGTTTGTGAGCTGGAATTCAATTGTGGCGTATTGATTC | |
| | CTTGCATCAGTCATTATTCCCTGCTGATTGACAGGTGAAAATTGGTTACGTTAAGTATTTCAT | |
| | ATGTTATATTGGCTGACATTTGCTTGCCTGCTCTTGTGTCAATATTGTTGTAAAGATCTCCAG | |
| | CTTTATGAGATAGCAATAGACACTGACTGTGGCTTTTGTGTGATGTTCCAGTGTTTTTCCTGA | |
| | CATAATTTAAGACATATTAAAAACCAGCAGCATCTTCCCTCTTGAGAAGCTTAATGCCAATAT | |
| | TATTGTCTTCCAGGGGAAGATCATGTATGCTCATAATCGGGTGCTAATTTCCACCAGTACGCT | |
| | CATGTTTAGGCATTAGGCACTATAACTGTAAAATTGAGCCTTCTTGATTGATTCATGTCAAGC | |
| | CTCATCTCGGCTCCTGCAGGGGAAGTCATCCGGCTGACCCTTTTTACACTAAAAGAAGAGATT | |
| | TGTGTTCCTTTCTTTCACCTGGAACCATCAAATTGACTGAATAATCTGTAATACATTAGTGCT | |
| | GACATTTGTTAGGGAGAATTAAACAAGACACAGTAATCATTCCCCAGAATAAAAATTGTGTTT | |
| | GATGGGCTGGGCATAAAAGTCAGGGCAGAGCCATCTATTGCTTACATTTGCTTCTG | |
| CNS-7 | TGAGACTGAGTGGGAGTGGGGATGAAGCCTTTTTTCTTCATTTTTTTTTCCTCAGGTGCAATT | 487 |
| (SEQ ID | CTGTGTTAATATAAGAGAAGTGTGGCCTTCCTTCTCATAGCACTAAAAGTGAGATAATCCCTG | |
| NO: 25) | TGTAAGAAATCAGTAAGTACGGTCTGCTTAATCTAGTCCCAGTGTGAAACTGTTGACATTTGT | |
| | TCTTTTTTCTATCATTATGTGACTGGGCCTGTTTTGTGCTGGATTAGGCACAAATCTCCTATG | |
| | CAGCACATTTGGCATGTTACTAGTAGTTTAACTTCATTAATAATGTATGAAGAAAATGTAATC | |
| | CATGACAAGGAAGCAAAGAAAAGTATTTTTTTTTTTTTTTGCTTCTCCCAAATCCTTTGGAAT | |
| | GAGTAATTATTCAACATTTTATGTTTGATGTTATATTTTACAATTCAACTTCCATAGGGCTGG | |
| | GCATAAAAGTCAGGGCAGAGCCATCTATTGCTTACATTTGCTTCTG | |
| CNS-8 | GCTGGTGCTTCTTTTTTCTGTTTTTTAAGCTGCTTTACAGACGAAAATGGAACTATATTTGGA | 593 |
| (SEQ ID | ACAATGCTTTCTGTTTTTCCATACTATTGATATTTGTGGAAAGTCACAAAATGGCCTAAGGAA | |
| NO: 26) | GCTAAGCTCGCCCCAAGCAGTGGTCACTTACAAGTACTTTTGTACTCTGTACTCCTGTCACAT | |
| | TTGGGCGATCAGAGCAACAGCTGGGGAGACTTTTTCAACAAAGATGAGTGTCAGATAATCCTG | |
| | ATGAGATTCCACATCCAACATCTTTTGTAATTATGTCACATTCAGCTGTAATGGAATAATTCA | |
| | AGCTGAAAGAACAAGCTTTGATCCTTTCTTAAACCTTTCCCTGTGGACTGGCTATCTAAAAGA | |
| | TTTAAAGATATTTCTGTTACAAGATCTAGTGTTTCCTCAGAGAAGTCATGCTTCTGAAGCATC | |
| | GTGATCTACAAGAACAATATCAAGTTTGCCAAACACATTTCTGAAAGCATCGTGTTTTGGGGG | |
| | GAGGGGTTGTATTTAATGAAGATATCAATAATATGCGGGCTGGGCATAAAAGTCAGGGCAGAG | |
| | CCATCTATTGCTTACATTTGCTTCTG | |

TABLE 2
Cis-regulatory elements (CRE) comprised in the promoters of Table 1

| Name | SEQUENCE |
|---|---|
| CRE0004_Lmx1b | CTGGGCAGAGAGGGGGCATCGGGGGCATGGCTAGGGGCCAGCACTGTGCT |
| (SEQ ID NO: 9) | TCCTGGGCGCCTCACCTCCTCCCTGACTCCTGGAGACTCCCAGCCCCTGT |
| | CTGGGAGATGAGCATTTAGGAATCTGCTTGTGCAGGGGTGGTGGGAGGGG |
| | CCGGGGTGGAGGGCGCATCCCCACGGGGAGATTGGATGGAAATGGCCTGC |
| | CAGTGTGTGTGTGAGTGTGCGCCTGTGGCAGCAGCAGAGTAAACAGCCGC |
| | TGCCCTGTCCTCTCTGCGGCCGTGGCCAGGTACAGGCCTGTTTGGACAGC |
| | TCCCTTGTCTGTCCGTCTGTTTGGGAGATGCTGGCTGATAGATGGGGATG |
| | GGCGGACTGTTAACCCCTCGTTGCCTGCACTGCTATGTGCTTCCTGCCTC |
| | ATCCATGGGGTAGAAGGTAGCCAGAAGGTGGTCCTGGCTGTGCCCCCAGC |
| | TCCTCTCTAGGGGGGAAACCTCTAGTTCTGAGTCAGGGACAGAGTGAGGA |
| | GGGCTCCAGGGCATCAAGAGCTTGCTCCTCCCCGCACCAGGGAGCCAAGG |
| | ACAGAGGAGAAGGGGGTCTTCCCCAGTGGTGACTAGGGGCAGAATATGTC |
| | TCTGAGTGAGTGTCTGGAGCCCTCCTCACCCCAACACCATG |
| CRE0003_Pitx3 | GGTGTGTGGAAGGGTGAGAGGCACACACAGACACTGAAAGAATCCTAGGC |
| (SEQ ID NO: 10) | CTGGTAGGCACTTAACAAATGTCTGTTACAGACCAGAATTTTATTGCTGT |
| | GAGAGACCCAAGCCCCTCATAGGAACAGTGAGAAACAGGTGCAGAAAGGC |
| | GGAGTAACTTTATCTAAAGTCATAGGCTCCCTGAATAGCAGAGCTGACAC |
| | CTACAAGGAAGCGTTGGAGACCAGATCTACCAGCTAGCCTCCCTGAGACC |
| | ACGAGGTGGCGCCGCAGCACCGGCTGTGGCCGATGCCAGCCAGGTAGCCG |
| | GTTTCCCACGTCCCCCGCACGCACGCACCTCTTTGCTGCAGGAATCCCGG |
| | GCTGCCCCGACCTGGAGTAGGGGGGGTGGTGAGTGGGACTGAGTCCCTAG |
| | AAGCCTGGACCCTCACTTCGTTCCTGTACATCCAGCTCGCCTGTAGACAG |
| | TGGGGGAGGATGAAGGGAAGAGGACTCAAGCGCAACTTTGAATCATCACG |
| | CCTTCGACAGTCCGCGCACGTTTATTTCATTTATCTTTGAAAACGAGGGA |
| | GGGGAAGCCTGGAGAAGGCGGGATGGGCCAAGGGTGAGTTGGCCCCCGGG |
| | GAGCTGGTCCCTGTTCCTGGCTTTAGTCCCAGGGGCGCGGTCTGTGTGTA |
| | GGGC |
| CRE0002_Gbf1 | TGCTACCAGAGCCGGGAGAGCTGCTCGGAGACGCCTCCGGGGTGCGGGCT |
| (SEQ ID NO: 11) | GGACATGAGCAGCGGCTGCCGGTCCTGGGACTAGGCCCCGCCATTTTGGA |
| | TCCGCTGACAGGTCAGCGAAGTCTCTTCCTAGAGTTCCGGTGTCGTGAAG |
| | GCCGCCCTGACATCGCAATAGGGAATTAGTGGGAAGGGCCCTTAAATTGG |
| | GCGAGCCAAGGTGGG |
| CRE0005_faf1_short | GGAACATTTAGACTTTGGGAAAGAGATTAGTGATTTACATTGCTATCTCA |
| (SEQ ID NO: 28) | CTGATTTAATTTAAATGCTCTTCCAAACCAAACACACATGTGCCGAAGAG |
| | GCTACTAAGAAACCCAACATGCAGAGTTCTCTATAAGTGCAGCCGACAGT |
| | GTTGACTGAAACTAAACTTGGAAATCCAGGGCACTAATGCACAATATCAA |
| | GCAATAAAACGGCATCTCTTTGGCAATATTTAATTTAAAAAAGAAGAAAG |
| | AGACAGGCGAAGATCAGGCACTGTCTGTTTTGGAGGATCAACCATTCTGC |
| | ATTTCAAAGCATTGGTCCCTGCAATATCCAGGTTACTGTGCTAGAATCTC |
| | GACTATTATATCGCAGTTGTGAGAGGGAGGGCAAAGATGTGTTTACTCAG |
| | TGATTAGGCCCTTAGAATAAGCCTCTAGCTCCTAGAGAGACAGCTCACCA |
| | CTTATTCATTTGGGCCAATTCACAAAGCCTAGGAAGATTAAACATCCATG |
| | CTGAGAAGACAAGCGAATGCAGACGGTGAAAAAGAAATAAAAATTCTTTA |
| | AAAACTCTGAGATGACTTCATTATTTTTCCACAAGGAAACTTTAGGAAAG |
| | TGTTTAGTTAGAGAAAAACCCACATTGACCTCTCTCTAAACCCTTAATCT |
| | TTCCTTTGTGGTGGCACTGCTTTGTGGTAAGCGACTGGCTCGCCTCGCCC |
| | CTCTTTTCACTGGAAGCTGAGAGAAAAAAGACTCTGGAGAAACAGTTTTC |
| | GTTCCAGGGACACAAACCCCTGACACTGTTAA |
| CRE0006_Pitx2_short | GAAAATTTAATGTCATTGAAGCTCTATTTCCAATAGCAAAGGAGCACTAT |
| (SEQ ID NO: 29) | TGCTAATAGACTTCAGAGCTTGAAATAAATAAATCTTTGGAATCCTGTTG |
| | CATCTCTTGGGGTGTGACATTTGACAGTCTTTTATAGCACAGAACGAAAC |
| | AAGTTTGTGAGCTGGAATTCAATTGTGGCGTATTGATTCCTTGCATCAGT |
| | CATTATTCCCTGCTGATTGACAGGTGAAAATTGGTTACGTTAAGTATTTC |
| | ATATGTTATATTGGCTGACATTGCTTGCCTGCTCTTGTGTCAATATTGTT |
| | GTAAAGATCTCCAGCTTTATGAGATAGCAATAGACACTGACTGTGGCTTT |
| | TGTGTGATGTTCCAGTGTTTTTCCTGACATAATTTAAGACATATTAAAAA |
| | CCAGCAGCATCTTCCCTCTTGAGAAGCTTAATGCCAATATTATTGTCTTC |
| | CAGGGGAAGATCATGTATGCTCATAATCGGGTGCTAATTTCCACCAGTAC |
| | GCTCATGTTTAGGCATTAGGCACTATAACTGTAAAATTGAGCCTTCTTGA |
| | TTGATTCATGTCAAGCCTCATCTCGGCTCCTGCAGGGGAAGTCATCCGGC |
| | TGACCCTTTTTACACTAAAAGAAGAGATTTGTGTTCCTTTCTTTCACCTG |
| | GAACCATCAAATTGACTGAATAATCTGTAATACATTAGTGCTGACATTTG |
| | TTAGGGAGAATTAAACAAGACACAGTAATCATTCCCCAGAATAAAAATTG |
| | TGTTTGAT |
| CRE0007_Pitx2_short | TGAGACTGAGTGGGAGTGGGGATGAAGCCTTTTTTCTTCATTTTTTTTTC |
| (SEQ ID NO: 30) | CTCAGGTGCAATTCTGTGTTAATATAAGAGAAGTGTGGCCTTCCTTCTCA |
| | TAGCACTAAAAGTGAGATAATCCCTGTGTAAGAAATCAGTAAGTACGGTC |
| | TGCTTAATCTAGTCCCAGTGTGAAACTGTTGACATTTGTTCTTTTTTCTA |
| | TCATTATGTGACTGGGCCTGTTTTGTGCTGGATTAGGCACAAATCTCCTA |
| | TGCAGCACATTTGGCATGTTACTAGTAGTTTAACTTCATTAATAATGTAT |
| | GAAGAAAATGTAATCCATGACAAGGAAGCAAAGAAAAGTATTTTTTTTTT |
| | TTTTTGCTTCTCCCAAATCCTTTGGAATGAGTAATTATTCAACATTTTAT |
| | GTTTGATGTTATATTTTACAATTCAACTTCCATA |
| CRE0008_Pitx2_short | GCTGGTGCTTCTTTTTTCTGTTTTTTAAGCTGCTTTACAGACGAAAATGG |
| (SEQ ID NO: 31) | AACTATATTTGGAACAATGCTTTCTGTTTTTCCATACTATTGATATTTGT |
| | GGAAAGTCACAAAATGGCCTAAGGAAGCTAAGCTCGCCCCAAGCAGTGGT |
| | CACTTACAAGTACTTTTGTACTCTGTACTCCTGTCACATTTGGGCGATCA |
| | GAGCAACAGCTGGGGAGACTTTTTCAACAAAGATGAGTGTCAGATAATCC |
| | TGATGAGATTCCACATCCAACATCTTTTGTAATTATGTCACATTCAGCTG |
| | TAATGGAATAATTCAAGCTGAAAGAACAAGCTTTGATCCTTTCTTAAACC |
| | TTTCCCTGTGGACTGGCTATCTAAAAGATTTAAAGATATTTCTGTTACAA |
| | GATCTAGTGTTTCCTCAGAGAAGTCATGCTTCTGAAGCATCGTGATCTAC |
| | AAGAACAATATCAAGTTTGCCAAACACATTTCTGAAAGCATCGTGTTTTG |
| | GGGGGAGGGGTTGTATTTAATGAAGATATCATTAATATGC |

TABLE 3
Minimal/Proximal Promoters comprised
in the promoters of Table 1

| Name | SEQUENCE |
|---|---|
| SYNP_CRE151 | GGGCTGGGCATAAAAGTCAGGGCAGAGCCATCT |
| (SEQ ID NO: 12) | ATTGCTTACATTTGCTTCTG |
| CRE0001v1_Pitx3 | GGGAGGATTGGAACAGAGACAAAAGGGAGGAGA |
| (SEQ ID NO: 13) | GACGGACAGCGACAAGTGGAGAAAATCGGCGAA |
| | ACTTGAGTGGCAGAGAAGTCTGAGCGCTGAGAC |
| | CCGGCGGCCCCGTGCGCCTTCCCACCTGGCGCC |
| | GATCCACTTTCCTCGGGGTAGCGGCCCAACCCA |
| | CTTCGCTGCCAGCCGATCCCTTTTACCCGTGGC |
| | TACCGGGACCACTCTACTCTCGCCCACTTGGCT |
| | CTGCCTAAGCGTCCTAGCCGGAGCGCGGTCTCT |
| | GCCACGTGGGGAGGGGCGCGGCCGAGTTGCTGA |
| | AGAGCGCTTCTGATTGGCCAGAGGGCGGGGTTC |
| | TTGGCGTCTCGCCGGCCAGACCCCTCCCTCAAA |
| | GGCGGGGCCTGGAGATCCACAGCTGGAAAGGGC |
| | GGAGCCCCAGCAGGGCAGCTGGAAAGGGGCGGG |
| | GCCTGACGCGCGCGGCTCGCCGCGGCGGGCTGG |
| | GGGCGCCCTGGTCTGCCATAAAGTGAATGGGCG |
| | CCGGCTGGGGGTGGCAGTACGCGGTGAGGCTCA |
| | CTCCCTCCGAGAGTCCAGGAGCGCC |

TABLE 4
Synthetic CNS-specific promoter overview

| | Minimal/proximal | | |
|---|---|---|---|
| Promoter name | promoter | CRE | UTR |
| CNS-1 | SYNP_CRE151 | CRE0004_Lmx1b | |
| CNS-2 | SYNP_CRE151 | CRE0003_Pitx3 | |
| CNS-3 | CRE0001v1_Pitx3 | CRE0002_Gbf1 | |
| CNS-4 | CRE0001_Pitx3 | | |
| CNS-5_v2 | CRE0005_faf1 | | |
| CNS-6_v2 | CRE0006_Pitx2 | | |
| CNS-7_v2 | CRE0007_Pitx2 | | |
| CNS-8_v2 | CRE0008_Pitx2 | | |
| CNS-1 + CMV-IE | SYNP_CRE151 | CRE0004_Lmx1b | CMV-IE UTR and |
| UTR and intron | | | intron |
| CNS-4 + CMV-IE | CRE0001_Pitx3 | | CMV-IE UTR and |
| UTR and intron | | | intron |
| CNS-5 | SYNP_CRE151 | CRE0005_faf1_short | |
| CNS-6 | SYNP_CRE151 | CRE0006_Pitx2_short | |
| CNS-7 | SYNP_CRE151 | CRE0007_Pitx2_short | |
| CNS-8 | SYNP_CRE151 | CRE0008_Pitx2_short | |

Synapsin-1
(SEQ ID NO: 14)
GAGGGCCCTGCGTATGAGTGCAAGTGGGTTTTAGGACCAGGATGAGGCG
GGGTGGGGGTGCCTACCTGACGACCGACCCCGACCCACTGGACAAGCAC
CCAACCCCCATTCCCCAAATTGCGCATCCCCTATCAGAGAGGGGGAGGG
GAAACAGGATGCGGCGAGGCGCGTGCGCACTGCCAGCTTCAGCACCGCG
GACAGTGCCTTCGCCCCCGCCTGGCGGCGCGCGCCACCGCCGCCTCAGC
ACTGAAGGCGCGCTGACGTCACTCGCCGGTCCCCCGCAAACTCCCCTTC
CCGGCCACCTTGGTCGCGTCCGCGCCGCCGCCGGCCCAGCCGGACCGCA
CCACGCGAGGCGCGAGATAGGGGGGCACGGGCGCGACCATCTGCGCTGC
GGCGCCGGCGACTCAGCGCTGCCTCAGTCTGCGGTGGGCAGCGGAGGAG
TCGTGTCGTGCCTGAGAGCGCAGTCG

1. A synthetic CNS-specific promoter comprising a sequence according to any one of SEQ ID NOs 1-8, 21-26 or a functional variant thereof.

2. The synthetic CNS-specific promoter of claim 1 comprising a sequence which is at least 60%, 65%, 70%, 75%, 80%, 90%, 95%, 96%, 97%, 98%, or 99% identical to any one of SEQ ID NOs 1-8, 21-26.

3. The synthetic CNS-specific promoter of any one of claim 1 or 2 wherein the functional variant of the synthetic CNS-specific promoter retains at least 25%, 50%, 75%, 80%, 85%, 80%, 95% or 100% of the activity of the reference promoter.

4. A CNS-specific cis-regulatory element (CRE) comprising a sequence according to any one of SEQ ID NOs: 9-11, 28-31, or a functional variant thereof.

5. The CNS-specific cis-regulatory elements (CRE) of claim 4 comprising a sequence which is at least 60%, 65%, 70%, 75%, 80%, 85%, 90%, 95%, 96%, 97%, 98% or 99% identical to any one of SEQ ID NOs: 9-11, 28-31.

6. A synthetic CNS-specific cis-regulatory module (CRM) comprising a CRE according to claim 4 or 5.

7. A synthetic CNS-specific promoter comprising a CRE according to claim 4 or 5 or a CRM according to claim 6.

8. An isolated minimal or proximal promoter comprising a sequence according to any one of SEQ ID NOs: 12-13, or a functional variant thereof.

9. The isolated minimal or proximal promoter according to claim 8 comprising a sequence which is at least 60%, 65%, 70%, 75%, 80%, 85%, 90%, 95%, 96%, 97%, 98% or 99% identical to any one of SEQ ID NOs: 12-13.

10. A synthetic CNS-specific promoter comprising a minimal or proximal promoter according to claim 8 or 9.

11. A synthetic CNS-specific promoter according to any one of claims 1-3, wherein the synthetic CNS-specific promoter comprises or consists of SEQ ID NO: 1 or SEQ ID NO: 21, or a functional variant thereof, and wherein the synthetic CNS-specific promoter is widely active in the brain when administered via intracerebroventricular (ICV) injection.

12. The synthetic CNS-specific promoter according to claim 11 wherein the synthetic CNS-specific promoter is active at a level at least 100%, 150% or 200% of the activity of Synapsin-1 (SEQ ID NO: 14) in the brain.

13. A synthetic CNS-specific promoter according to any one of claims 1-3, wherein the synthetic CNS-specific promoter comprises or consists of SEQ ID NO: 8 or SEQ ID NO: 26 and wherein the synthetic CNS-specific promoter is active in the midbrain when administered via intravenous (IV) injection.

14. The synthetic CNS-specific promoter according to claim 13, wherein the synthetic CNS-specific promoter is active in dopaminergic neurones.

15. An expression cassette comprising a synthetic CNS-specific promoter according to any one of claim 1, 2, 3, 7, 10, 11, 12, 13 or 14 operably linked to a nucleic acid sequence encoding an expression product.

16. A vector comprising a synthetic CNS-specific promoter according to any one of claim 1, 2, 3, 7, 10, 11, 12, 13 or 14 or an expression cassette according to claim 15.

17. The vector of claim 16 which is a viral vector, e.g. an AAV vector, an adenoviral vector, a retroviral vector or a lentiviral vector.

18. The vector of claim 17, wherein the vector is a lentiviral vector.

19. The vector of claim 17, wherein the vector is an AAV vector.

20. A virion comprising a vector according to any one of claim 17, 18 or 19.

21. A pharmaceutical composition comprising a synthetic CNS-specific promoter according to any one of claim 1, 2, 3, 7, 10, 11, 12, 13 or 14, an expression cassette according to claim 15, a vector according to claim 16, 17, 18 or 19, or a virion according to claim 20.

22. A synthetic CNS-specific promoter according to any one of claim 1, 2, 3, 7, 10, 11, 12, 13 or 14, an expression cassette according to claim 15, a vector according to claim 16, 17, 18 or 19, a virion according to claim 20, or a pharmaceutical composition according to claim 21 for use as a medicament.

23. A cell comprising a synthetic CNS-specific promoter according to any one of claim 1, 2, 3, 7, 10, 11, 12, 13 or 14, an expression cassette according to claim 15, a vector according to claim 16, 17, 18 or 19, or a virion according to claim 20.

24. A synthetic CNS-specific promoter according to any one of claim 1, 2, 3, 7, 10, 11, 12, 13 or 14, an expression cassette according to claim 15, a vector according to claim 16, 17, 18 or 19, a virion according to claim 20, or a pharmaceutical composition according to claim 21 for use in the manufacture of a pharmaceutical composition for the treatment of a medical condition or disease.

25. A method for producing an expression product, the method comprising providing a synthetic CNS-specific expression cassette according to claim 15 in a CNS cell and expressing the expression product present in the synthetic CNS-specific expression cassette.

26. The method of claim 25, wherein the synthetic CNS-specific expression cassette comprises or consists of SEQ ID NO: 1 or SEQ ID NO: 21, or a functional variant thereof, and wherein the expression product is widely expressed in the brain when the expression cassette is provided via ICV injection.

27. The method of claim 26, wherein the CNS-specific expression cassette drives expression at a level of at least 100%, 150% or 200% compared to the activity of Synapsin-1 (SEQ ID NO: 14) in the brain.

28. The method of claim 25, wherein the synthetic CNS-specific expression cassette comprises or consists of SEQ ID NO: 8 or SEQ ID NO: 26, or a functional variant thereof, and wherein the expression product is expressed in the midbrain when administered via intravascular injection.

29. The method of claim 28, wherein the expression product is expressed dopaminergic neurones.

30. A method of expressing a therapeutic transgene in a CNS cell, the method comprising introducing into the CNS cell a synthetic CNS-specific expression cassette according to claim 15, a vector according to claim 16, 17, 18 or 19, or a virion according to claim 20.

31. The method of expressing a therapeutic transgene in a CNS cell according to claim 30, wherein the expression cassette, the vector or the virion are introduced by intravenous injection.

32. The method of claim 31, wherein the injection is in one of the cephalic, median or basilic veins.

33. A method of therapy of a subject in need thereof, preferably a human, the method comprising:
administering to the subject an expression cassette according to claim 15, a vector according to claim 16, 17, 18 or 19, a virion according to claim 20, or a pharmaceutical composition according to claim 21, which comprises a sequence encoding a therapeutic product operably linked to a promoter according any one of claim 1, 2, 3, 7, 10, 11, 12, 13 or 14; and
expressing a therapeutic amount of the therapeutic product in the CNS of said subject.

34. A method of expressing an expression product in a dopaminergic neurone, the method comprising introducing into the dopaminergic neurone a synthetic CNS-specific expression cassette according to claim 15 via intravascular injection, wherein the CNS-specific expression cassette comprises SEQ ID NO: 8 or SEQ ID NO: 26, or a functional variant thereof.
